(19) United States
(12) Patent Application Publication
(10) Pub. No.: US 2023/0398214 A1
(43) Pub. Date: Dec. 14, 2023

(54) ANTI-VIRAL CENTRAL MEMORY CD8+ VETO CELLS IN HAPLOIDENTICAL STEM CELL TRANSPLANTATION

(71) Applicants: Yeda Research and Development Co. Ltd., Rehovot (IL); Board of Regents, The University of Texas System, Austin, TX (US)

(72) Inventors: Yair REISNER, Houston, TX (US); Esther BACHAR-LUSTIG, Rehovot (IL); Assaf LASK, Rehovot (IL); Noga OR-GEVA, Rehovot (IL); Rotem GIDRON BUDOVSKY, Rehovot (IL); Rakefet SIDLIK MUSKATEL, Rehovot (IL)

(73) Assignees: Yeda Research and Development Co. Ltd., Rehovot (IL); Board of Regents, The University of Texas System, Austin, TX (US)

(21) Appl. No.: 17/633,294

(22) PCT Filed: Aug. 6, 2020
(86) PCT No.: PCT/IL2020/050865

§ 371 Date: Feb. 7, 2022

Related U.S. Application Data
(60) Provisional application No. 62/883,164, filed on Aug. 6, 2019.

(51) Int. Cl. A61K 39/00; C12N 5/0783; A61K 31/664; A61P 37/06

(52) U.S. Cl. CPC A61K 39/4622; C12N 5/0636; A61K 39/4633; A61K 39/4614; A61K 39/464411; A61K 39/46444; A61K 39/464838; A61K 31/664; A61P 37/06; A61K 39/464429; A61K 39/4621; C12N 2501/2307; C12N 2501/2315; C12N 2501/2321; C12N 2502/1121

(57) ABSTRACT

Methods of generating an isolated population of non-graft versus host disease (GVHD) inducing cells comprising a central memory T-lymphocyte (Tcm) phenotype, the cells being tolerance inducing cells and/or endowed with anti-disease activity, and capable of homing to the lymph nodes following transplantation, are disclosed. Cells generated by the methods, pharmaceutical compositions and methods of treatment are also disclosed.

### RELATED APPLICATION/S

This application claims the benefit of priority of U.S. Provisional Patent Application No. 62/883,164 filed on Aug. 6, 2019, the contents of which are incorporated herein by reference in their entirety.

### FIELD AND BACKGROUND OF THE INVENTION

The present invention, in some embodiments thereof, relates to veto cells generated from memory T cells and, more particularly, but not exclusively, to methods of their manufacture and use in therapy for attaining a stable and long term cell or tissue transplantation.

The paradigm that high dose chemoradiotherapy “conditioning” is critical for attaining durable remission with allogeneic hematopoietic cell transplantation (allo-HCT) for hematological malignancies has shifted to emphasize the immune graft-vs-malignancy effect of the newly formed, donor derived immune system, in conjunction with subsequent immunotherapy. However, reduction of the conditioning, which retains a more robust host immune system, capable of protecting the patient from lethal infection during the early period post-transplant, is also associated with enhanced risk for graft rejection. In Haploidentical HCT, this can be overcome by using T cell replete transplants, as the alloreactive T cells in the graft are capable of attacking host T cells and paving the way for engraftment of the donor stem cells. However, such alloreactive T cells are also associated with life-threatening graft-versus-host (GVH) reactivity, requiring use of potent immunosuppressive drugs post-transplant. Some progress has been made in recent years in reducing the severity of graft-versus-host disease (GVHD) using high dose cyclophosphamide (CY) following transplant. Nevertheless, chronic GVHD remains a problem and prolonged use of immuno-suppressive drugs is required, which adversely impacts graft-vs-malignancy effects. Furthermore, the occurrence of GVHD in many patients is associated with poor thymic function, further limiting adequate anti-tumor immunity.

To overcome this obstacle, ‘megadose’ T cell depleted haploidentical HCT may be used, which is free of GVHD risk even in the absence of post-transplant immune suppressive therapy. However, achieving engraftment after non-myeloablative (NMA) conditioning remains a major challenge due to the high level of anti-donor T cell clones surviving the pre-transplant conditioning regimen. One approach used to address this challenge is the use of high dose CY shortly following transplant (discussed in PCT publication nos. WO 2013/093920 and WO 2013/093919).

Furthermore, various approaches have been contemplated for generation of tolerance inducing cells (e.g. veto cells) devoid of GVH reactivity and the use of same as an adjuvant treatment for graft transplantation, some are summarized infra.

PCT Publication No. WO 2001/49243 discloses the use of non-alloreactive anti-third party cytotoxic T-lymphocytes (CTLs), wherein the non-alloreactive anti-third party CTLs are generated by directing T-lymphocytes of the donor against a third party antigen or antigens (in the absence of exogenous IL-2), the dose being substantially depleted of T-lymphocytes (e.g. CD4⁺ T cells and/or CD56⁺ natural killer cells) capable of developing into alloreactive CTLs.

PCT Publication No. WO 2007/023491 discloses the use of tolerogenic cells for reducing or preventing graft rejection of a non-syngeneic graft in a subject. The tolerogenic T regulatory cells disclosed (e.g. CD4⁺CD25⁺ cells) may be derived from any donor who is non-syngeneic with both the subject and the graft (“third-party” tolerogenic cells). The graft (e.g. bone marrow) may be derived from any graft donor who is allogeneic or xenogeneic with the subject.

PCT Publication No. WO 2010/049935 discloses the use of non-GVHD inducing anti-third party cells having a central memory T-lymphocyte (Tcm) phenotype, the cells being tolerance-inducing cells and capable of homing to the lymph nodes following transplantation. According to WO 2010/049935 the cells are generated by contacting non-syngeneic peripheral blood mononuclear cells (PBMC) with a third party antigen or antigens in a culture deprived of cytokines and then culturing the cells in the presence of IL-15 under conditions which allow proliferation of cells comprising the Tcm phenotype.

PCT Publication No. WO 2012/032526 discloses the use of non-GVHD inducing anti-third party cells having a Tcm phenotype, the cells being tolerance-inducing cells and capable of homing to the lymph nodes following transplantation, in disease treatment. According to WO 2012/032526 the cells are generated by contacting PBMCs with a third party antigen or antigens in the presence or absence of IL-21 under conditions which allow elimination of GVH reactive cells (e.g. culturing for 1-5 days) and then culturing the cells in the presence of IL-15 under conditions which allow proliferation of cells comprising the Tcm phenotype.

PCT Publication No. WO 2013/035099 discloses the use of non-GVHD inducing anti-third party cells having a central memory T-lymphocyte (Tcm) phenotype, the cells being tolerance-inducing cells and/or endowed with anti-disease activity, and capable of homing to the lymph nodes following transplantation. According to WO 2013/035099 the cells are generated by contacting PBMCs with a third party antigen or antigens in the presence of IL-21 so as to allow enrichment of antigen reactive cells and then culturing the cells in the presence of IL-21, IL-15 and IL-7 in an antigen free environment so as to allow proliferation of cells comprising the Tcm phenotype.

PCT Publication No. WO 2018/002924 discloses veto cells generated from memory T cells, methods of their manufacture and use in transplantation and in disease treatment. According to WO 2018/002924 the veto cells are non-GVHD inducing anti-third party cells having a Tcm phenotype, are tolerance-inducing cells and/or endowed with anti-disease activity, and are capable of homing to the lymph nodes following transplantation.

Additional background art includes PCT publication nos: WO 2017/009852, WO 2017/009853 and WO2018/134824.

### SUMMARY OF THE INVENTION

According to an aspect of some embodiments of the present invention there is provided a method of generating an isolated population of non-graft versus host disease (GVHD) inducing cells comprising a central memory T-lymphocyte (Tcm) phenotype, the cells being tolerance inducing cells and/or endowed with anti-disease activity, and capable of homing to the lymph nodes following transplantation, the method comprising:
(a) contacting a first population of peripheral blood mononuclear cells (PBMCs) from a donor subject with an antibody capable of binding CD14⁺ expressing cells and selecting CD14⁺ expressing cells capable of maturing into antigen presenting cells;
(b) loading the antigen presenting cells with viral peptides;
(c) treating a second population of PBMCs of the same donor subject as the first population of PBMCs with one or more agents capable of depleting CD4⁺, CD56⁺ and CD45RA⁺ expressing cells so as to obtain a population of cells comprising memory T cells expressing a CD45RA⁻CD8⁺ phenotype;
(d) contacting the population of cells comprising the memory T cells with the antigen presenting cells loaded with the viral peptides of step (b) in the presence of IL-21 so as to allow enrichment of viral reactive memory T cells; and
(e) culturing the cells resulting from step (d) in the presence of IL-21, IL-15 and/or IL-7 so as to allow proliferation of cells comprising the Tcm phenotype, thereby generating the isolated population of non-GVHD inducing cells comprising the Tcm phenotype.

According to an aspect of some embodiments of the present invention there is provided a method of generating an isolated population of non-graft versus host disease (GVHD) inducing cells comprising a central memory T-lymphocyte (Tem) phenotype, the cells being tolerance inducing cells and/or endowed with anti-disease activity, and capable of homing to the lymph nodes following transplantation, the method comprising:
(a) providing a population of cells comprising T cells, wherein the T cells in the population of cells comprise at least 40% memory T cells expressing CD45RA⁻CD8⁺ phenotype and depleted of CD4⁺, CD56⁺ and CD45RA⁺ expressing cells; and
(b) contacting the population of cells comprising the memory T cells with viral peptides derived from 4-10 types of viruses, wherein at least one of said viruses comprises a BK virus, in the presence of IL-21 so as to allow enrichment of viral reactive memory T cells; and
(c) culturing the cells resulting from step (b) in the presence of IL-21, IL-15 and/or IL-7 so as to allow proliferation of cells comprising the Tcm phenotype, thereby generating the isolated population of non-GVHD inducing cells comprising the Tcm phenotype.

According to an aspect of some embodiments of the present invention there is provided a method of generating an isolated population of non-graft versus host disease (GVHD) inducing cells comprising a central memory T-lymphocyte (Tcm) phenotype, the cells being tolerance inducing cells and/or endowed with anti-disease activity, and capable of homing to the lymph nodes following transplantation, the method comprising:
(a) contacting a first population of peripheral blood mononuclear cells (PBMCs) from a donor subject with an antibody capable of selecting CD14⁺ expressing cells and selecting CD14⁺ expressing cells capable of maturing into dendritic cells;
(b) loading the dendritic cells with viral peptides derived from 4-10 types of viruses, wherein at least one of said viruses comprises a BK virus;
(c) treating a second population of PBMCs of the same donor subject as the first population of PBMCs with one or more agents capable of depleting CD4⁺, CD56⁺ and CD45RA⁺ expressing cells so as to obtain a population of cells comprising memory T cells expressing a CD45RA⁻CD8⁺ phenotype;
(d) contacting the population of cells comprising the memory T cells with the dendritic cells loaded with the viral peptides of step (b) in the presence of IL-21 so as to allow enrichment of viral reactive memory T cells; and
(e) culturing the cells resulting from step (d) in the presence of IL-21, IL-15 and/or IL-7 so as to allow proliferation of cells comprising the Tcm phenotype, thereby generating the isolated population of non-GVHD inducing cells comprising the Tcm phenotype.

According to an aspect of some embodiments of the present invention there is provided an isolated population of non-GVHD inducing cells comprising cells having a central memory T-lymphocyte (Tcm) phenotype, the cells being tolerance inducing cells and/or endowed with anti-disease activity, and capable of homing to the lymph nodes following transplantation, generated according to the method of some embodiments of the invention.

According to an aspect of some embodiments of the present invention there is provided a pharmaceutical composition comprising as an active ingredient the isolated population of non-GVHD inducing cells of some embodiments of the invention and a pharmaceutical acceptable carrier.

According to an aspect of some embodiments of the present invention there is provided a method of treating a disease in a subject in need thereof, the method comprising administering to the subject a therapeutically effective amount of the isolated population of non-GVHD inducing cells of some embodiments of the invention, thereby treating the disease in the subject.

According to an aspect of some embodiments of the present invention there is provided a use of the isolated population of non-GVHD inducing cells of some embodiments of the invention for the manufacture of a medicament identified for treating a disease in a subject in need thereof.

According to an aspect of some embodiments of the present invention there is provided a method of reducing graft rejection and/or graft versus host disease (GVHD) and/or inducing donor specific tolerance in a subject in need thereof, wherein the subject is in need of a non-syngeneic cell or tissue transplant, the method comprising administering to the subject a therapeutically effective amount of the isolated population of non-GVHD inducing cells of some embodiments of the invention, thereby reducing graft rejection and/or GVHD in the subject.

According to an aspect of some embodiments of the present invention there is provided a use of the isolated population of non-GVHD inducing cells of some embodiments of the invention for the manufacture of a medicament identified for reducing graft rejection and/or graft versus host disease (GVHD) and/or inducing donor specific tolerance in a subject in need thereof, wherein the subject is in need of the non-syngeneic cell or tissue transplant.

According to an aspect of some embodiments of the present invention there is provided a method of treating a subject in need of a non-syngeneic cell or tissue transplant, the method comprising:
(a) transplanting a cell or tissue transplant into the subject; and
(b) administering to the subject a therapeutically effective amount of the isolated population of non-GVHD inducing cells of some embodiments of the invention, thereby treating the subject in need of the cell or tissue transplant.

According to an aspect of some embodiments of the present invention there is provided a use of the isolated population of non-GVHD inducing cells of some embodiments of the invention for the manufacture of a medicament identified as an adjuvant treatment for a non-syngeneic cell or tissue transplant into a subject, wherein the subject is in need of the non-syngeneic cell or tissue transplant.

According to an aspect of some embodiments of the present invention there is provided a method of treating a subject in need of an immature hematopoietic cell transplantation, the method comprising:
(a) conditioning the subject under non-myeloablative pre-transplant conditioning protocol, wherein the non-myeloablative conditioning comprises a total body irradiation (TBI) and a chemotherapeutic agent, wherein the TBI and the chemotherapeutic agent are administered on days −7 to −1 prior to transplantation (e.g. on days −7 to −2, e.g. on days −6 to −1, prior to transplantation);
(b) transplanting into the subject a dose of T cell depleted immature hematopoietic cells, wherein the T cell depleted immature hematopoietic cells comprises less than 5×10⁵ CD3⁺ T cells per kilogram ideal body weight of the subject, and wherein the T cell depleted immature hematopoietic cells comprise at least 5×10⁶ CD34⁺ cells per kilogram ideal body weight of the subject;
(c) administering to the subject a therapeutically effective amount of cyclophosphamide, wherein the therapeutically effective amount of the cyclophosphamide comprises 25-200 mg cyclophosphamide per kilogram ideal body weight of the subject, and wherein the therapeutically effective amount of the cyclophosphamide is to be administered to the subject in two doses 3 and 4 days following the transplantation of the T cell depleted immature hematopoietic cells; and
(d) administering to the subject a therapeutically effective amount of the isolated population of non-GVHD inducing cells of some embodiments of the invention, wherein the isolated population of non-GVHD inducing cells are administered on day 6-9 following the transplantation of the T cell depleted immature hematopoietic cells.

thereby treating the subject in need of the immature hematopoietic cell transplantation.

According to some embodiments of the invention, the first population of the PBMCs and the second population of the PBMCs are from the same batch.

According to some embodiments of the invention, the memory T cells expressing the CD45RA⁻CD8⁺ phenotype constitute at least 40% of T cells in the population of cells.

According to some embodiments of the invention, the agent is an antibody.

According to some embodiments of the invention, the contacting in the presence of IL-21 is affected for 12 hours to 6 days.

According to some embodiments of the invention, the contacting in the presence of IL-21 is affected for 3 days.

According to some embodiments of the invention, the culturing in the presence of any of IL-21, IL-15 and/or IL-7 is affected for 6 to 12 days.

According to some embodiments of the invention, the culturing in the presence of any of IL-21, IL-15 and/or IL-7 is affected for 9 days.

According to some embodiments of the invention, the culturing further comprises adding glucose to a concentration of at least 50 mg/dl.

According to some embodiments of the invention, the viral peptides are presented on antigen presenting cells.

According to some embodiments of the invention, the antigen presenting cells are obtained by a method comprising contacting PBMCs from a donor subject with an antibody capable of binding CD14⁺ expressing cells and selecting CD14⁺ expressing cells capable of maturing into antigen presenting cells.

According to some embodiments of the invention, the antigen presenting cells are of the same donor subject as the population of cells comprising the memory T cells.

According to some embodiments of the invention, the selecting CD14⁺ expressing cells is not affected by plastic adherence.

According to some embodiments of the invention, the antigen presenting cells comprise dendritic cells.

According to some embodiments of the invention, the dendritic cells are mature dendritic cells.

According to some embodiments of the invention, the dendritic cells are obtained by a method comprising culturing the CD14⁺ expressing cells in a culture comprising dendritic cell maturation factors.

According to some embodiments of the invention, the viral peptides are derived from 1-10 types of viruses.

According to some embodiments of the invention, the viral peptides are derived from 2-10 types of viruses.

According to some embodiments of the invention, the viral peptides are derived from 4-10 types of viruses.

According to some embodiments of the invention, at least one of the viral peptides comprises at least one peptide from a BK virus. According to some embodiments of the invention, the viral peptides comprise an Epstein-Barr virus (EBV) peptide, a cytomegalovirus (CMV) peptide, a BK Virus peptide and an Adenovirus (Adv) peptide.

According to some embodiments of the invention, the viral peptides comprise at least one of EBV-LMP2, EBV-BZLF1, EBV-EBNA1, EBV select, CMV-pp65, CMV-IE-1, Adv-penton, Adv-hexon, BKV LT, BKV (capsid VP1), BKV (capsid protein VP2), BKV (capsid protein VP2, isoporm VP3), BKV (small T antigen).

According to some embodiments of the invention, the viral peptides comprise at least one of AdV5 Hexon, hCMV pp65, EBV select and BKV LT.

According to some embodiments of the invention, the viral peptides comprise AdV5 Hexon, hCMV pp65, EBV select and BKV LT.

According to some embodiments of the invention, the viral peptides further comprise at least one type of a bacterial peptide, a fungal peptide, or a tumor peptide.

According to some embodiments, the viral peptides and at least one type of a bacterial peptide, a fungal peptide, or a tumor peptide are utilized together.

According to some embodiments, the viral peptides and at least one type of a bacterial peptide, a fungal peptide, or a tumor peptide are utilized separately.

According to some embodiments of the invention, the PBMCs are non-syngeneic with respect to a subject.

According to some embodiments of the invention, the PBMCs are allogeneic with respect to a subject.

According to some embodiments of the invention, the population of cells comprising the memory T cells is non-syngeneic with respect to a subject.

According to some embodiments of the invention, the population of cells comprising the memory T cells is allogeneic with respect to a subject.

According to some embodiments of the invention, the cells having the Tcm phenotype comprise a CD3⁺, CD8⁺, CD62L⁺, CD45RA⁻, CD45RO⁺ signature.

According to some embodiments of the invention, the cells having the Tcm phenotype comprise at least 30% of the isolated population of non-GVHD inducing cells.

According to some embodiments of the invention, the isolated population of non-GVHD inducing cells are formulated for administration as fresh cells.

According to some embodiments of the invention, the isolated population of non-GVHD inducing cells are formulated for administration as cryopreserved cells.

According to some embodiments of the invention, the method further comprises transplanting a cell or tissue transplant into the subject.

According to some embodiments of the invention, the medicament further comprises a cell or tissue transplant.

According to some embodiments of the invention, the method further comprises administering to the subject genetically modified T cells.

According to some embodiments of the invention, the medicament further comprises genetically modified T cells.

According to some embodiments of the invention, the genetically modified T cells comprise CAR-T cells or TCR-transgenic T cells.

According to some embodiments of the invention, the cell or tissue transplant comprises immature hematopoietic cells.

According to some embodiments of the invention, the disease is a malignant disease.

According to some embodiments of the invention, the malignant disease is a solid tumor or tumor metastasis.

According to some embodiments of the invention, the malignant disease is a hematological malignancy.

According to some embodiments of the invention, the malignant disease is selected from the group consisting of a leukemia, a lymphoma, a myeloma, a melanoma, a sarcoma, a neuroblastoma, a colon cancer, a colorectal cancer, a breast cancer, an ovarian cancer, an esophageal cancer, a synovial cell cancer, a hepatic cancer, a pancreatic cancer, and a metastasis or a relapse.

According to some embodiments of the invention, the disease is a non-malignant disease.

According to some embodiments of the invention, the non-malignant disease is selected from the group consisting of an organ dysfunction or failure, a hematologic disease, a graft related disease, an infectious disease, a genetic disease or disorder, a sickle cell disease, an autoimmune disease and a metabolic disorder.

According to some embodiments of the invention, the non-malignant disease is selected from the group consisting of an aplastic anemia, a severe immune deficiency and a non-malignant bone marrow failure.

According to some embodiments of the invention, the isolated population of non-GVHD inducing cells is for administration following the cell or tissue transplant (e.g. non-syngeneic cell or tissue transplant).

According to some embodiments of the invention, the isolated population of non-GVHD inducing cells is for administration on day 6-9 following the cell or tissue transplant (e.g. non-syngeneic cell or tissue transplant).

According to some embodiments of the invention, the isolated population of non-GVHD inducing cells is for administration on day 7 following the cell or tissue transplant (e.g. non-syngeneic cell or tissue transplant).

According to some embodiments of the invention, the isolated population of non-GVHD inducing cells is for administration at a dose of at least 2.5×10⁶ CD8⁺ cells per kg ideal body weight.

According to some embodiments of the invention, the non-syngeneic cell or tissue transplant comprises immature hematopoietic cells.

According to some embodiments of the invention, the immature hematopoietic cells comprise T cell depleted immature hematopoietic cells.

According to some embodiments of the invention, the immature hematopoietic cells comprise at least 5×10⁶ CD34⁺ cells per kilogram ideal body weight of the subject.

According to some embodiments of the invention, the immature hematopoietic cells are depleted of CD3⁺ and/or CD19⁺ expressing cells.

According to some embodiments of the invention, the immature hematopoietic cells comprise less than 4×10⁵ CD3⁺ expressing cells per kg ideal body weight of the subject.

According to some embodiments of the invention, the immature hematopoietic cells comprise less than 3×10⁵ CD3⁺ expressing cells per kg ideal body weight of the subject.

According to some embodiments of the invention, the immature hematopoietic cells comprise less than 2×10⁵ CD3⁺ expressing cells per kg ideal body weight of the subject.

According to some embodiments of the invention, the method further comprises conditioning the subject under non-myeloablative conditioning protocol prior to the transplanting.

According to some embodiments of the invention, the method further comprises a non-myeloablative pre-transplant conditioning protocol.

According to some embodiments of the invention, the non-myeloablative conditioning protocol comprises at least one of total body irradiation (TBI), a partial body irradiation (TLI), a chemotherapeutic agent, an antibody immunotherapy or a co-stimulatory blockade.

According to some embodiments of the invention, the TBI comprises a single or fractionated irradiation dose within the range of 1-5 Gy.

According to some embodiments of the invention, TBI is administered on day −1 prior to transplantation.

According to some embodiments of the invention, TBI is administered on the day of transplantation, e.g. in the morning of day 0, and transplantation is carried out on the same day, e.g. in the evening.

According to some embodiments of the invention, the chemotherapeutic agent comprises at least one of Fludarabine, Cyclophosphamide, Rapamycin, Busulfan, Trisulphan, Melphalan or Thiotepa.

According to some embodiments of the invention, the non-myeloablative conditioning protocol comprises T cell debulking.

According to some embodiments of the invention, the non-myeloablative conditioning protocol does not comprise T cell debulking.

According to some embodiments of the invention, the T cell debulking is affected by at least one of anti-thymocyte globulin (ATG) antibodies, anti-CD52 antibodies or anti-CD3 (OKT3) antibodies.

According to some embodiments of the invention, the method further comprises administering to the subject a therapeutically effective amount of cyclophosphamide subsequent to the transplanting.

According to some embodiments of the invention, the further comprises a therapeutically effective amount of cyclophosphamide, wherein the cyclophosphamide is to be administered to the subject following the non-syngeneic cell or tissue transplant.

According to some embodiments of the invention, the therapeutically effective amount of the cyclophosphamide comprises 25-200 mg cyclophosphamide per kilogram ideal body weight of the subject.

According to some embodiments of the invention, the therapeutically effective amount of the cyclophosphamide is to be administered to the subject in two doses 3 and 4 days post-transplant.

According to some embodiments of the invention, the non-myeloablative pre-transplant conditioning protocol comprises T cell debulking.

According to some embodiments of the invention, the T cell debulking is affected by anti-thymocyte globulin (ATG).

According to some embodiments of the invention, the non-myeloablative pre-transplant conditioning protocol does not comprise T cell debulking.

According to some embodiments of the invention, corticosteroids are not administered.

According to some embodiments of the invention, the subject is not treated chronically with GVHD prophylaxis following transplantation.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow diagram outlining processing and testing of anti-viral CD8⁺ veto cell depleted cells according to some embodiments of the invention.

FIG. 2 is a flow diagram outlining processing and testing of CD34⁺ enriched and CD3⁺CD19⁺ depleted cells according to some embodiments of the invention.

FIG. 3 is a schematic representation of a conditioning regimen of a recipient subject with fresh immature hematopoietic cell transplant for use according to some embodiments of the invention.

FIG. 4 is a schematic representation of a conditioning regimen of a recipient subject with cryopreserved immature hematopoietic cell transplant for use according to some embodiments of the invention.

FIG. 5 is a schematic illustration of a combination protocol combining mis-matched megadose T cell depleted hematopoietic stem cell transplant with post-transplant high dose CY, following non-myeloablative conditioning by T cell debulking (with ATG), Fludarabine and 3 GY TBI.

FIG. 6 is a graph illustrating that infusion of veto cells on day 7 enables to overcome graft rejection in the absence of T cell debulking of the recipients. C3H recipient mice were transplanted with megadose T cell depleted Balb/Nude (H2^{d}) BM cells following 3.5 GY TBI. Transplanted mice also received either CY post-transplant (100 mg/kg) on day 3-4 (designated Nu/BN+CTX) or 5×10⁶ Balb/c veto Tcm (H2^{d}) on day 7 post-transplant (designated Nu/BN+Tcm D-7), or both (designated Nu/BN+Tcm D7⁺CTX). Percentage of donor cells in peripheral blood was analyzed by FACS using anti-donor and anti-host (H2^{d} and H2^{k}) antibodies. Each dot represents one mouse belonging to the appropriate group.

FIG. 7 is a schematic illustration of a treatment protocol without ATG which is replaced by administration of veto cells on day 7.

FIGS. 8A-C illustrate the anti-viral activity of Tcm cells as determined by intracellular staining of INF-γ and TNF-α. Veto-Tcm cells were co-cultured with the respective viral peptide mix (EBV, CMV, Adeno, BKV) in the presence of Brefeldin A (eBioscience) at 37° C. 5% CO₂ for 6 hrs. Cells were fixed, permeabilized (Invitrogen Fix & Perm set), and immunostained for CD45, CD3, CD8, IFNγ, and TNFα (BD). Positive control included TCR-independent stimulation with PMA/Ionomycin. Cells were gated on a CD45⁺/FSC lymphocyte gate and CD3⁺CD8⁺.

### DESCRIPTION OF SPECIFIC EMBODIMENTS OF THE INVENTION

The present invention, in some embodiments thereof, relates to veto cells generated from memory T cells and, more particularly, but not exclusively, to methods of their manufacture and use in therapy for attaining a stable and long term cell or tissue transplantation.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, the present inventors have uncovered an improved protocol for generating large scale doses of tolerance inducing cells (e.g. veto cells) which also comprise an anti-disease activity (e.g. anti-viral activity) without inducing a graft versus host (GVH) reaction. These cells were generated by producing viral presenting dendritic cells (by way of selection of CD14⁺ cells from PBMCs, culturing in maturation factors and loading with specific combination of viral peptides) and then depleting alloreactive clones from memory CD8 T cells by way of a specific combination of viral peptides used for viral antigen activation. The present inventors have further uncovered that supplementing T cell depleted megadose allogeneic hematopoietic cell transplantation (allo-HCT) with these graft-facilitating veto cells (e.g. administered after HCT, e.g. on day +7 post HCT) can tolerize host anti-donor T cells, enable engraftment in the absence of GVHD following safe, non-myeloablative (NMA) conditioning (e.g. comprising TBI and a chemotherapeutic agent such as Fludarabine), with minimal post-transplant immune suppressive therapy comprising cyclophosphamide (e.g. administered on days +3 and +4 post HCT).

Briefly, non-mobilized peripheral blood mononuclear cells (PBMC) were obtained from a cell donor and fractionated using Ficoll to obtain mononuclear cells (MNC). The MNC were separated into two fractions, one for generating stimulator cells (i.e. antigen presenting cells presenting viral peptides) and the second for generating anti-viral central memory CD8⁺ veto cells. Accordingly, stimulator cells were generated from the first MNC fraction by selecting CD14⁺ expressing cells from the MNC fraction using CD14-binding monoclonal antibodies, the CD14⁺ expressing cells were grown with dendritic cell maturation factors comprising IL-4, GM-CSF, IFN-γ and LPS for 16 hours. The mature dendritic cells (mDCs) were then loaded with viral peptides comprising EBV, CMV, BKV and Adenovirus, and were irradiated (e.g. by 25 Gy). Concomitantly, memory cells were enriched from the second MNC fraction by negative selection of CD4⁻CD56-CD45RA⁻ expressing cells using CD4⁺, CD56⁺ and CD45RA⁺ antibodies conjugated to super-paramagnetic particles. The CD4⁻CD56⁻CD45RA⁻ depleted cells were co-cultured with the viral-loaded dendritic cells (at a ratio of 5:1 cells to DC) for 3 days in a T cell growth media deprived of cytokines and supplemented with only IL-21 (such conditions allow proliferation of viral reactive memory CD8 T cells and elimination of GVH reactive cells). After 3 days, the T cells were cultured in the presence of IL-21, IL-15 and IL-7 for an additional 9 days (such conditions allow proliferation of cells comprising the Tcm phenotype). The cell medium was replaced every 2 days and the glucose and PH levels were monitored and adjusted as needed. The resulting population of cells comprise anti-viral central memory CD8⁺ veto cells as determined by immunophenotyping (e.g. by expression of CD3⁺CD8⁺CD62L⁺CD45RO⁺) and functional immune studies (e.g. by assessing the anti-viral activity, for example, by restimulating the Tcm cells against the original viral peptides used at the beginning of the culture and measuring the percentage of INFγ⁺TNF⁺ cells e.g. by FACS).

The anti-viral central memory CD8⁺ veto cells facilitate engraftment of allogeneic CD34⁺ enriched CD3⁺CD19⁺ depleted hematopoietic cell transplantation (i.e. megadose T cell depleted allogeneic hematopoietic stem cell (HSC) transplant) following a non-myeloablative (NMA) conditioning regimen. Thus, for example, the recipient subject may be treated daily with fludarabine on days −6 to −3 (or on days −7 to −4) followed by low dose TBI on day −1 (or on day −2) (i.e. on days 6 to 3, days 7 to 4, day 1, or day 2, respectively, prior to transplantation) as the preparative regimen. On days +3 and +4 (i.e. days 3 to 4 after transplantation), the patient receives cyclophosphamide (CY) followed by the infusion of the veto cells on day +7 (i.e. day +7 after transplantation). Optionally, ATG can be administered daily on days on days −9 to −7 (or on days −10 to −8) prior to transplantation so as to induce T cell debulking in the recipient subject.

Taken together, the anti-viral central memory CD8⁺ veto cells offer disease treatment (e.g. protection from viral infections or treatment of viral infections) coupled with enhanced engraftment of donor hematopoietic progenitors, by virtue of their ability to induce immune tolerance towards donor cells. Moreover, the veto cells enable engraftment of megadose T cell depleted allogeneic HSC transplant without prolonged immune suppression following a safe non-myeloablative regimen. Furthermore, the large scale protocol provided enables generation of anti-viral central memory CD8⁺ veto cells in doses suitable for use in treatment of human subjects.

Thus, according to one aspect of the present invention there is provided a method of generating an isolated population of non-graft versus host disease (GVHD) inducing cells comprising a central memory T-lymphocyte (Tcm) phenotype, the cells being tolerance inducing cells and/or endowed with anti-disease activity, and capable of homing to the lymph nodes following transplantation, the method comprising:
(a) contacting a first population of peripheral blood mononuclear cells (PBMCs) from a donor subject with an antibody capable of binding CD14⁺ expressing cells and selecting CD14⁺ expressing cells capable of maturing into antigen presenting cells;
(b) loading the antigen presenting cells with viral peptides;
(c) treating a second population of PBMCs of the same donor subject as the first population of PBMCs with one or more agents capable of depleting CD4⁺, CD56⁺ and CD45RA⁺ expressing cells so as to obtain a population of cells comprising memory T cells expressing a CD45RA⁻CD8⁺ phenotype;
(d) contacting the population of cells comprising the memory T cells with the antigen presenting cells loaded with the viral peptides of step (b) in the presence of IL-21 so as to allow enrichment of viral reactive memory T cells; and
(e) culturing the cells resulting from step (d) in the presence of IL-21, IL-15 and/or IL-7 so as to allow proliferation of cells comprising the Tcm phenotype, thereby generating the isolated population of non-GVHD inducing cells comprising the Tcm phenotype.

According to one aspect of the present invention there is provided a method of generating an isolated population of non graft versus host disease (GVHD) inducing cells comprising a central memory T-lymphocyte (Tcm) phenotype, the cells being tolerance inducing cells and/or endowed with anti-disease activity, and capable of homing to the lymph nodes following transplantation, the method comprising:
(a) providing a population of cells comprising T cells, wherein the T cells in the population of cells comprise at least 40% memory T cells expressing a CD45RA⁻CD8⁺ phenotype and depleted of CD4⁺, CD56⁺ and CD45RA⁺ expressing cells; and
(b) contacting the population of cells comprising the memory T cells with viral peptides derived from 4-10 types of viruses, wherein at least one of the viruses comprises a BK virus, in the presence of IL-21 so as to allow enrichment of viral reactive memory T cells; and
(c) culturing the cells resulting from step (b) in the presence of IL-21, IL-15 and/or IL-7 so as to allow proliferation of cells comprising the Tcm phenotype, thereby generating the isolated population of non-GVHD inducing cells comprising the Tcm phenotype.

The term “isolated” refers to cells which have been isolated from their natural environment (e.g., the human body).

According to one embodiment, a population of cells refers to a heterogeneous cell mixture.

The term “non-graft versus host disease” or “non-GVHD” as used herein refers to having substantially reduced or no graft versus host (GVH) inducing reactivity. Thus, the cells of some embodiments of the present invention are generated as to not significantly cause graft versus host disease (GVHD) as evidenced by survival, weight and overall appearance of the transplanted subject 30-120 days following transplantation. Methods of evaluating a subject for reduced GVHD are well known to one of skill in the art.

According to one embodiment, the cells of some embodiments of the present invention have at least 10%, at least 20%, at least 30%, at least 40%, at least 50%, at least 55%, at least 60%, at least 65%, at least 70%, at least 75%, at least 80%, at least 85%, at least 90%, at least 95% or even 100% reduced reactivity against a host relative to cells not generated according to the present teachings.

The phrase “central memory T-lymphocyte (Tcm) phenotype” as used herein refers to a subset of T cells which home to the lymph nodes. Cells having the Tcm phenotype, in humans, typically comprise a CD3⁺/CD8⁺/CD62L⁺/CD45RO⁺/CD45RA⁻ signature. It will be appreciated that Tem cells may express all of the signature markers on a single cell or may express only part of the signature markers on a single cell. Determination of a cell phenotype can be carried out using any method known to one of skill in the art, such as for example, by Fluorescence-activated cell sorting (FACS) or capture ELISA labeling.

According to one embodiment, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, at least 99%, or even 100% of the isolated population of non-GVHD inducing cells have the Tcm cell signature.

According to a specific embodiment, cells having the Tcm phenotype comprise 20% of the isolated population of non-GVHD inducing cells.

According to a specific embodiment, cells having the Tcm phenotype comprise 30% of the isolated population of non-GVHD inducing cells.

According to a specific embodiment, cells having the Tcm phenotype comprise 40% of the isolated population of non-GVHD inducing cells.

According to a specific embodiment, cells having the Tcm phenotype comprise 50% of the isolated population of non-GVHD inducing cells.

According to a specific embodiment, cells having the Tcm phenotype comprise 60% of the isolated population of non-GVHD inducing cells.

According to a specific embodiment, cells having the Tcm phenotype comprise 70% of the isolated population of non-GVHD inducing cells.

According to a specific embodiment, about 10-20%, about 10-30%, about 10-40%, about 10-50%, about 20-30%, about 20-40%, about 30-50%, about 40-60%, about 50-70%, about 60-80%, about 70-90%, about 80-100%, or about 90-100% of the isolated population of non-GVHD inducing cells have the Tcm cell signature.

According to a specific embodiment, cells having the Tcm phenotype comprise 10-30% of the isolated population of non-GVHD inducing cells.

According to a specific embodiment, cells having the Tcm phenotype comprise 10-50% of the isolated population of non-GVHD inducing cells.

According to a specific embodiment, cells having the Tcm phenotype comprise 20-40% of the isolated population of non-GVHD inducing cells.

According to a specific embodiment, cells having the Tcm phenotype comprise 30-50% of the isolated population of non-GVHD inducing cells.

According to a specific embodiment, cells having the Tcm phenotype comprise 50-70% of the isolated population of non-GVHD inducing cells.

The non-GVHD inducing cells comprising a Tcm phenotype of the invention are also referred to herein as “Tcm cells”.

As mentioned, Tcm cells typically home to the lymph nodes following transplantation. According to some embodiments, the isolated population of cells of some embodiments of the present invention may home to any of the lymph nodes following transplantation, as for example, the peripheral lymph nodes and mesenteric lymph nodes. The homing nature of these cells allows them to exert their veto effect in a rapid and efficient manner.

The non-GVHD inducing cells comprising a Tcm phenotype of some embodiments of the present invention are tolerance-inducing cells.

The phrase “tolerance inducing cells” as used herein refers to cells which provoke decreased responsiveness of the recipient's cells (e.g. recipient's T cells) when they come in contact with the donor cells as compared to the responsiveness of the recipient's cells in the absence of administered tolerance inducing cells. Tolerance inducing cells include veto cells (i.e. T cells which lead to apoptosis of host T cells upon contact with same) as was previously described in PCT Publication Nos. WO 2001/049243 and WO 2002/102971.

The term “veto activity” relates to immune cells (e.g. donor derived T cells) which lead to inactivation of anti-donor recipient T cells upon recognition and binding to the veto cells. According to one embodiment, the inactivation results in apoptosis of the anti-donor recipient T cells.

The non-GVHD inducing cells comprising a Tcm phenotype of the invention are also referred to herein as “veto cells”.

Additionally or alternatively, the isolated population of non-GVHD inducing cells comprising a Tcm phenotype of some embodiments of the present invention comprises anti-disease activity.

The term “anti-disease activity” refers to the function of the Tcm cells against a diseased cell. The anti-disease activity may be directly against a diseased cell, e.g. killing capability of the diseased cell. This activity may be due to TCR independent killing mediated by LFA1-I/CAM1 binding [Arditti et al., Blood (2005) 105(8):3365-71. Epub 2004 Jul. 6]. Additionally or alternatively, the anti-disease activity may be indirect, e.g. by activation of other types of cells (e.g. CD4⁺ T cells, B cells, monocytes, macrophages, NK cells) which leads to death of the diseased cell (e.g. by killing, apoptosis, or by secretion of other factors, e.g. antibodies, cytokines, etc.).

The term “anti-viral activity” refers to the function of the Tcm cells against a virally infected cell (e.g. expressing viral antigen/s in the context of MHC-peptide complex on the cell surface). Typically the anti-viral activity results in killing of the virally infected cell.

The term “anti-tumor activity” refers to the function of the Tcm cells against a tumor cell. Typically the anti-tumor activity results in killing of the tumor cell. According to a specific embodiment, anti-tumor activity comprises graft versus leukemia/lymphoma (GVL) activity.

A diseased cell may comprise, for example, a virally infected cell, a bacterial infected cell, a cancer cell [e.g. cell of a solid tumor or leukemia/lymphoma cell, also referred to herein as graft versus leukemia (GVL) activity of the Tcm cells], a cell associated with an autoimmune disease, a cell associated with an allergic response, or a cell altered due to stress, radiation or age.

According to some embodiments, the non-GVHD inducing cells of some embodiments of the present invention comprising a Tcm phenotype may be non-genetically modified cells or genetically modified cells (e.g. cells which have been genetically engineered to express or not express specific genes, markers or peptides or to secrete or not secrete specific cytokines) depending on the application needed (e.g. on the disease to be treated). Such determinations are well within the ability of one of ordinary skill in the art.

According to one embodiment, the Tcm cells express a chimeric antigen receptor (CAR) or a modified T cell receptor (TCR).

Accordingly, the Tcm cells of some embodiments of the invention may be transduced to express a TCR or a CAR.

As used herein “transduction with a TCR” refers to cloning of two chains (i.e., polypeptide chains), such as, an alpha chain of a T cell receptor (TCR), a beta chain of a TCR, a gamma chain of a TCR, a delta chain of a TCR, or a combination thereof (e.g. αβ chains or γδ chains). According to one embodiment, the TCR comprises the variable region of a TCR (e.g. α- and β-chains or γ- and δ-chains). Method of transducing cells (e.g. T cells) with a TCR (e.g. to generated TCR-T cells) are known in the art and are disclosed e.g. in Nicholson et al. Adv Hematol. 2012; 2012:404081; Wang and Rivière Cancer Gene Ther. 2015 March; 22(2):85-94); and Lamers et al., Cancer Gene Therapy (2002) 9, 613-623.

As used herein “transduction with a CAR” refers to cloning of a nucleic acid sequence encoding a chimeric antigen receptor (CAR), wherein the CAR comprises an antigen recognition moiety and a T-cell activation moiety. A chimeric antigen receptor (CAR) is an artificially constructed hybrid protein or polypeptide containing an antigen binding domain of an antibody (e.g., a single chain variable fragment (scFv)) linked to T-cell signaling or T-cell activation domains. Method of transducing cells (e.g. T cells) with a CAR (e.g. to generate CAR-T) are known in the art and are disclosed e.g. in Davila et al. Oncoimmunology. 2012 Dec. 1; 1(9):1577-1583; Wang and Rivière Cancer Gene Ther. 2015 March; 22(2):85-94); and Maus et al. Blood. 2014 Apr. 24; 123(17):2625-35.

According to one embodiment, the genetically modified Tcm cells express a transgenic TCR (TCR-T) or a CAR (CAR-T).

Thus, the isolated population of non-GVHD inducing cells comprising a Tcm phenotype of some embodiments of the invention may be genetically modified for therapy (e.g. anti-cancer cell therapy) by redirecting the cell specificity (e.g. T cell specificity) by promoting presentation of a receptor targeting a disease antigen (e.g. tumor antigen) by way of transducing with a T cell receptor (TCR) or a chimeric antigen receptor (CAR). For example, TCR or CAR expressing Tcm cells may be generated to target malignant diseases e.g. follicular lymphoma (CD20 or GD2), neuroblastoma (CD171), non-Hodgkin lymphoma (CD20), lymphoma (CD19), glioblastoma (IL13Rα2), chronic lymphocytic leukemia or CLL and acute lymphocytic leukemia or ALL (both CD19).

According to some embodiments of the invention there is provided a method of generating the isolated population of non-GVHD inducing cells comprising a Tcm phenotype.

According to one embodiment the method comprises contacting a first population of peripheral blood mononuclear cells (PBMCs) from a donor subject with an antibody capable of binding CD14⁺ expressing cells and selecting CD14⁺ expressing cells capable of maturing into antigen presenting cells.

The term “donor subject” refers to a human being.

According to one embodiment, the donor subject is non-syngeneic with respect to a subject (e.g. allogeneic), as discussed in detail hereinbelow.

The term “peripheral blood mononuclear cells (PBMCs)” refers to a fraction of a blood sample comprising lymphocytes (including T cells, B cells, NK cells, etc.) and monocytes.

According to one embodiment, the PBMCs are non-syngeneic with respect to a recipient subject (e.g. allogeneic), as discussed in detail hereinbelow.

According to one embodiment, the PBMCs used for generation of the non-GVHD inducing cells comprising the Tcm phenotype are non-mobilized (i.e. unprimed) PBMCs, i.e. cells not obtained by means of using drugs to affect the movement of hematopoietic precursors (e.g., stem cells) from bone marrow into peripheral blood circulation.

According to one embodiment, the PBMCs are collected 5-15 days (e.g. 7-10 days, e.g. 7 days, e.g. 8 days) prior to the planned transplant date of the hematopoietic cells (HCT) (i.e. Day 0), as discussed in detail hereinbelow. However, it is to be understood that PBMCs can be collected at any time prior to the planned transplant date. Such PBMCs can be stored as is, or can be treated as discussed below and then stored for future use (e.g. cryopreserved).

According to one embodiment, PBMCs are collected from a donor subject using standard techniques. According to a specific embodiment, PBMCs are collected using leukapheresis, i.e. a process which essentially removes PBMCs from a donor subject, returning the remaining blood components to the donor subject.

According to one embodiment, the PBMC collection procedure (e.g. leukapheresis) yields e.g. 0.01-1000×10¹⁰ mononuclear cells (MNC), e.g. 0.1-100×10¹⁰, e.g. 1×10¹⁰ MNC.

According to one embodiment, the PBMC collection procedure (e.g. leukapheresis) yields a minimum of 500×10⁶ mononuclear cells (MNC), e.g. 500×10¹⁰ to 1000×10¹⁰ MNC, e.g. 1×10¹⁰ MNC.

According to one embodiment, the PBMCs are collected in a single collection procedure.

According to one embodiment, the PBMCs are collected in a two, three, four, five or more collection procedure (e.g. in order to obtain the required number of MNC).

According to one embodiment, if the PBMCs are collected (e.g. from the same donor subject) in two or more collection procedures, the PBMCs may be pooled together (e.g. for further processing) or used separately.

According to one embodiment, any of the aforementioned collections may be referred to as a batch. Alternatively, any group of collections (e.g. from the same donor subject in the context of collection over several days e.g. 1, 2, 3, 4, 5 days, e.g. 3 days) may be referred to as a batch.

According to one embodiment, any of the aforementioned collections of PBMCs may be kept in a collection tube for one or more days (e.g. e.g. 1, 2, 3, 4, 5 days, e.g. 1-2 days) prior to further processing (e.g. MNC isolation as discussed below).

According to one embodiment, mononuclear cells (MNC) are isolated from the PBMCs. Any standard technique known in the art can be used for MNC isolation from PBMCs. For example, according to one embodiment, the collected PBMCs are diluted (e.g. at 1:2) with Dulbecco's Phosphate-Buffered Saline (DPBS), e.g. without Calcium and Magnesium and e.g. supplemented with e.g. 0.6% ACD-A and 0.5% of 25% HAS)), and the MNC are isolated by ficoll density gradient separation. After ficoll density gradient separation, the MNC preparation of one embodiment is platelet washed (i.e. thrombowashed), e.g. 1-5 times, e.g. 1-3 times e.g. twice, by manual centrifugation and is resuspended with Wash Buffer (e.g. PBS with ACD-A and 0.5% of 25% HSA).

The PBMC preparation or MNC preparation of some embodiments of the invention is divided into two fractions (e.g. equal fractions). One PBMC or MNC fraction is further processed into antigen presenting cells (i.e. referred to herein as first population of PBMCs) and the second PBMC or MNC fraction (i.e. referred to herein second population of PBMCs) is enriched for CD8⁺ memory T cells.

According to one embodiment, the first population of PBMCs and the second population of PBMCs are from the same batch.

Alternatively, two PBMC preparations may be used from different PBMC collection procedures. Thus, according to one embodiment, the first population of PBMCs and the second population of PBMCs are from diverse batches.

According to one embodiment, antigen presenting cells (e.g. dendritic cells) are generated by first contacting the first population of PBMCs with an antibody capable of binding CD14⁺ expressing cells and selecting CD14⁺ expressing cells.

According to one embodiment, the antibody capable of binding CD14⁺ expressing cells is a CD14 monoclonal antibody. Such antibodies can be obtained commercially e.g. from BD Biosciences, Santa Cruz Biotechnology and R&D Systems.

Selecting CD14⁺ expressing cells using CD14 monoclonal antibodies may be carried out using any method known in the art, such as by the use of magnetic-activated cell sorting (MACS™) available from e.g. Miltenyi Biotec, FACS sorter and/or capture ELISA labeling.

According to one embodiment, different depletion/separation methods can be combined, for example, magnetic cell sorting can be combined with FACS, to increase the separation quality or to allow sorting by multiple parameters.

According to one embodiment, selection of CD14⁺ expressing cells is not affected by plastic adherence.

According to one embodiment, the CD14⁺ expressing cells are selected by magnetic separation techniques. Different magnetic beads are available from a number of sources, including for example, Dynal (Norway), Advanced Magnetics (Cambridge, MA, U.S.A.), Immuncon (Philadelphia, U.S.A.), Immunotec (Marseille, France), Invitrogen, Stem cell Technologies (U.S.A), Cellpro (U.S.A) and Miltenyi Biotec GmbH (Germany). Alternatively, antibodies can be biotinylated or conjugated with digoxigenin and used in conjunction with avidin or anti-digoxigenin coated affinity columns.

According to one embodiment, the CD14 monoclonal antibodies are conjugated to magnetic particles.

According to a specific embodiment, the magnetic particles comprise super-paramagnetic iron dextran particles.

According to a specific embodiment, the CD14 labeled cells are processed on CliniMACS® column.

According to a specific embodiment, the CD14 labeled cells are selected on SuperMACS™ II using the XS Separation Column.

According to one embodiment, the CD14 magnetically labeled cells (i.e. CD14⁺ expressing cells) are retained by the separation column (i.e. positive selection) and the CD14⁻ cells are removed. The CD14⁺ cells are then released from the column and collected. According to one embodiment, samples from each fraction are removed for cell count, viability and/or immunophenotyping.

According to one embodiment, viability is assessed by positive expression of 7AAD, i.e. 7AAD⁺ cells.

According to one embodiment, the CD14⁺ enriched cell preparation is resuspended at a cell concentration of e.g. 1-10×10⁶ cells/ml, e.g. 3×10⁶ cells/ml, in cell culture medium (e.g. dendritic cell culture medium, e.g. CellGro/1% HSA). According to one embodiment, the cell culture medium is supplemented with cytokines and growth factors. Determination of cytokines and growth factors to be used is within the skill of a person of skill in the art. For example, the cell culture medium is supplemented with IL-4 (e.g. 200-2000 IU/mL, e.g. 1000 IU/mL) and GM-CSF (e.g. 1000-4000 IU/mL, e.g. 2000 IU/mL). The cell suspension is then seeded (e.g. in cell culture plates e.g. Cell Factory plates) and incubated for 12-36 hours, e.g. for 16-24 hours, e.g. for 24 hours, in at 37° C., 5% CO₂.

According to one embodiment, in order to induce maturation of the CD14⁺ expressing cells into antigen presenting cells (e.g. dendritic cells), the CD14⁺ enriched cell preparation is cultured in the presence of maturation factors (e.g. dendritic cell maturation factors). Determination of maturation factors to be used is within the skill of a person of skill in the art. Thus, according to one embodiment, the seeded (e.g. in cell culture plates, e.g. Cell Factory plates) CD14⁺ enriched cells are cultured in the presence of IL-4 (e.g. 200-2000 IU/mL, e.g. 1000 IU/mL), GM-CSF (e.g. 1000-4000 IU/mL, e.g. 2000 IU/mL), LPS (e.g. 10-100 ng/mL, e.g. 40 ng/mL), and IFN-7 (e.g. 50-500 IU/mL, e.g. 200 IU/mL) for 10-24 hours, e.g. for 14-18 hours, e.g. for 16 hours, in at 37° C., 5% CO₂.

After the culturing period, the antigen presenting cells (e.g. mature dendritic cells i.e. mDCs) are obtained from the cell culture. According to one embodiment, non-adherent cells are removed and the antigen presenting cells (i.e. adherent cells) are detached from the culture plates using any method known in the art (e.g. by adding ice-cold buffer e.g. ACD-A with 0.5% of 25% HAS and DPBS Buffer and incubation on ice or frozen gel packs for 10-60 minutes, e.g. 30 minutes). The harvested antigen presenting cells are then centrifuged, washed and resuspended (e.g. in ACD-A with 0.5% of 25% HAS and DPBS Buffer).

According to one embodiment, antigen presenting cells (e.g. mDCs) are loaded with an antigen or antigens.

As used herein the phrase “loading” refers to the attachment of an antigen or antigens (e.g. peptides) to MHC peptides (e.g. MHC class I or II) presented in the peptide-MHC complex on the surface of the antigen-presenting cell (APC).

As used herein the phrase “antigen or antigens” refers to a soluble or non-soluble (such as membrane associated) molecule capable of inducing an immune response.

For example, an antigen or antigens can be whole cells (e.g. live or dead cells), cell fractions (e.g. lysed cells), cell antigens (e.g. cell surface antigens), a protein extract, a purified protein or a synthetic peptide.

According to one embodiment, the antigen or antigens comprise viral antigens.

According to one embodiment, the viral antigens are derived from at least 1, 2, 3, 4, 5, 6, 7, 8, 9, 10, 15, 20 or more different types of viruses.

According to one embodiment, the viral antigens are derived from 1-50, 1-40, 1-30, 1-25, 1-20, 1-15, 1-10, 1-9, 1-8, 1-7, 1-6, 1-5, 1-4, 1-3, 1-2, 2-50, 2-40, 2-30, 2-20, 2-10, 2-8, 2-6, 2-4, 3-50, 3-40, 3-30, 3-20, 3-10, 3-9, 3-7, 3-5, 3-4, 4-50, 4-40, 4-30, 4-20, 4-10, 4-8 or 4-6 types of viruses.

According to a specific embodiment, the viral antigens are derived from 1-20 types of viruses.

According to a specific embodiment, the viral antigens are derived from 1-10 types of viruses.

According to a specific embodiment, the viral antigens are derived from 1-4 types of viruses.

According to a specific embodiment, the viral antigens are derived from 2-10 types of viruses.

According to a specific embodiment, the viral antigens are derived from 2-4 types of viruses.

According to a specific embodiment, the viral antigens are derived from 4-20 types of viruses.

According to a specific embodiment, the viral antigens are derived from 4-10 types of viruses.

According to a specific embodiment, the viral antigens are derived from 4-8 types of viruses.

According to a specific embodiment, the viral antigens are derived from 4-6 types of viruses.

Exemplary viruses from which antigens can be derived (i.e. originated from) include, but are not limited to, Epstein-Barr virus (EBV), Adenovirus (Adv), cytomegalovirus (CMV), cold viruses, flu viruses, hepatitis A, B, and C viruses, herpes simplex, HIV, influenza, Japanese encephalitis, measles, polio, rabies, respiratory syncytial, rubella, smallpox, varicella zoster, rotavirus, West Nile virus, Polyomavirus (e.g. BK Virus), zika virus, parvovirus (e.g. parvovirus B19), varicella-zoster virus (VZV), Herpes simplex virus (HSV), severe acute respiratory syndrome (SARS), and severe acute respiratory syndrome coronavirus 2 (SARS-CoV-2).

Particular examples of viruses and their respective antigens include, but are not limited to, BK Virus antigens include, but are not limited to, BKV LT; BKV (capsid VP1), BKV (capsid protein VP2), BKV (capsid protein VP2, isoporm VP3), BKV (small T antigen); Adenovirus antigens include, but are not limited to, Adv-penton or Adv-hexon; CMV antigens include, but are not limited to, envelope glycoprotein B, CMV IE-1 and CMV pp65, UL28, UL32, UL36, UL40, UL48, UL55, UL84, UL94, UL99 UL103, UL151, UL153, US 29, US 32; EBV antigens include, but are not limited to, EBV LMP2, EBV BZLF1, EBV EBNA1, EBV P18, and EBV P23; hepatitis antigens include, but are not limited to, the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens include, but are not limited to, immediate early proteins and glycoprotein D; HIV antigens include, but are not limited to, gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens include, but are not limited to, hemagglutinin and neuraminidase; Japanese encephalitis viral antigens include, but are not limited to, proteins E, M-E, M-E-NS1, NS1, NS1-NS2A and 80% E; measles antigens include, but are not limited to, the measles virus fusion protein; rabies antigens include, but are not limited to, rabies glycoprotein and rabies nucleoprotein; respiratory syncytial viral antigens include, but are not limited to, the RSV fusion protein and the M2 protein; rotaviral antigens include, but are not limited to, VP7sc; rubella antigens include, but are not limited to, proteins E1 and E2; Severe acute respiratory syndrome (SARS-CoV) antigens include, but are not limited to, S1, RBD, Nuclecapsid and Plpro; severe acute respiratory syndrome coronavirus 2 (SARS-CoV-2) antigens include, but are not limited to, S1, S2, S1+S2 ECD, RBD, N antigen, S antigen and nuclecapsid; and varicella zoster viral antigens include, but are not limited to, gpl and gpll.

According to one embodiment, the antigen or antigens comprise viral peptides (or fragments thereof).

According to one embodiment, the viral peptides comprise 1-50, 1-40, 1-30, 1-25, 1-20, 1-15, 1-10, 1-9, 1-8, 1-7, 1-6, 1-5, 1-4, 1-3, 1-2, 2-50, 2-40, 2-30, 2-20, 2-10, 2-8, 2-6, 2-4, 3-50, 3-40, 3-30, 3-20, 3-10, 3-9, 3-7, 3-5, 3-4, 4-50, 4-40, 4-30, 4-20, 4-10, 4-8 or 4-6 viral peptides.

According to a specific embodiment, the viral peptides comprise 4-50 viral peptides (e.g. in a single formulation or in several formulations).

According to a specific embodiment, the viral peptides comprise 4-40 viral peptides (e.g. in a single formulation or in several formulations).

According to a specific embodiment, the viral peptides comprise 4-30 viral peptides (e.g. in a single formulation or in several formulations).

According to a specific embodiment, the viral peptides comprise 4-20 viral peptides (e.g. in a single formulation or in several formulations).

According to a specific embodiment, the viral peptides comprise 4-10 viral peptides (e.g. in a single formulation or in several formulations).

According to a specific embodiment, the viral peptides comprise 4-8 viral peptides (e.g. in a single formulation or in several formulations).

According to a specific embodiment, the viral peptides comprise 4-6 viral peptides (e.g. in a single formulation or in several formulations).

According to one embodiment, the viral peptides comprise 1, 2, 3, 4, 5, 6, 7, 8, 9, 10, 11, 12, 13, 14, 15, 16, 17, 18, 19, 20, 25, 30, 35, 40, 45 or 50 viral peptides (e.g. in a single formulation or in several formulations).

According to a specific embodiment, the viral peptides comprise 4 viral peptides (e.g. in a single formulation or in several formulations).

According to a specific embodiment, the viral peptides comprise 5 viral peptides (e.g. in a single formulation or in several formulations).

According to a specific embodiment, the viral peptides comprise 6 viral peptides (e.g. in a single formulation or in several formulations).

According to a specific embodiment, the viral peptides comprise 8 viral peptides (e.g. in a single formulation or in several formulations).

According to a specific embodiment, the viral peptides comprise 10 viral peptides (e.g. in a single formulation or in several formulations).

According to a specific embodiment, the viral peptides comprise 15 viral peptides (e.g. in a single formulation or in several formulations).

According to a specific embodiment, the viral peptides comprise 20 viral peptides (e.g. in a single formulation or in several formulations).

According to a specific embodiment, the viral peptides comprise 30 viral peptides (e.g. in a single formulation or in several formulations).

According to a specific embodiment, the viral peptides comprise 40 viral peptides (e.g. in a single formulation or in several formulations).

According to a specific embodiment, the viral peptides comprise 50 viral peptides (e.g. in a single formulation or in several formulations).

According to a specific embodiment, the viral peptides comprise peptides from a single organism (i.e. from one virus type).

According to a specific embodiment, the viral peptides comprise peptides from two or more organism (i.e. a mixture from 2, 3, 4, 5 or more virus types).

According to one embodiment, the viral peptides comprise a BK virus peptide.

According to a specific embodiment, the viral peptides comprise at least one of an Epstein-Barr virus (EBV) peptide, a cytomegalovirus (CMV) peptide, a BK Virus peptide and an Adenovirus (Adv) peptide.

According to a specific embodiment, the viral peptides comprise an Epstein-Barr virus (EBV) peptide, a cytomegalovirus (CMV) peptide, a BK Virus peptide and an Adenovirus (Adv) peptide.

According to a specific embodiment, the viral peptides comprise at least one of EBV-LMP2, EBV-BZLF1, EBV-EBNA1, EBV-BRAF1, EBV-BMLF1, EBV-GP340/350 EBNA2, EBV-EBNA3a, EBV-EBNA3b, EBV-EBNA3c, CMV-pp65, CMV-IE-1, Adv-penton, Adv-hexon, BKV LT, BKV (capsid VP1), BKV (capsid protein VP2), BKV (capsid protein VP2, isoporm VP3), and BKV (small T antigen).

According to a specific embodiment, the viral peptides comprise at least one of AdV5 Hexon, hCMV pp65, EBV select (discussed below) and BKV LT.

Dedicated software can be used to analyze antigen sequences to identify immunogenic short peptides, i.e., peptides presentable in context of major histocompatibility complex (MHC) class I or MHC class II.

According to a specific embodiment, the antigen or antigens comprise a mixture of pepmixes which are overlapping peptide libraries (e.g. 15mers overlapping by 11 amino acids) spanning the entire protein sequence of three viruses: CMV, EBV, and Adeno (such pepmixes can be commercially bought e.g. from JPT Technologies, Berlin, Germany).

According to a specific embodiment, the viral peptides comprise “EBV select” i.e. a commercial product from Miltenyi Biotec comprising 43 MHC class 1 and class 2 restricted peptides from 13 different proteins from EBV (e.g. MACS GMP PepTivator® EBV Select, e.g. catalog no. 170-076-143). Additionally or alternatively, the viral peptides comprise “collection EBV” i.e., a commercial product from JPT have comprising a pepmix which includes peptides from 14 different EBV antigens. Additionally or alternatively, the viral peptides comprise PepMix™ BKV (capsid protein VP1), PepMix™ BKV (capsid protein VP2), PepMix™ BKV (capsid protein VP2, isoform VP3), PepMix™ BKV (large T antigen), PepMix™ BKV (small T antigen), commercially available from JPT.

According to another specific embodiment, the antigen or antigens comprise a mixture of seven pepmixes spanning EBV-LMP2, EBV-BZLF1, EBV-EBNA1, CMV-pp65, CMV-IE-1, Adv-penton and Adv-hexon at a concentration of e.g. 100 ng/peptide or 700 ng/mixture of the seven peptides.

According to one embodiment, the antigen or antigens comprise antigen or antigens of an infectious organism (e.g., bacterial, fungal organism) which typically affects immune comprised subjects, such as transplantation patients.

According to one embodiment, the antigen is a bacterial antigen, such as but not limited to, an antigen of anthrax; gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus and tetanus.

As further particular examples of bacterial antigens, anthrax antigens include, but are not limited to, anthrax protective antigen; gram-negative bacilli antigens include, but are not limited to, lipopolysaccharides; haemophilus influenza antigens include, but are not limited to, capsular polysaccharides; diptheria antigens include, but are not limited to, diptheria toxin; Mycobacterium tuberculosis antigens include, but are not limited to, mycolic acid, heat shock protein 65 (HSP65), the 30 kDa major secreted protein and antigen 85A; pertussis toxin antigens include, but are not limited to, hemagglutinin, pertactin, FIM2, FIM3 and adenylate cyclase; pneumococcal antigens include, but are not limited to, pneumolysin and pneumococcal capsular polysaccharides; rickettsiae antigens include, but are not limited to, rompA; streptococcal antigens include, but are not limited to, M proteins; and tetanus antigens include, but are not limited to, tetanus toxin.

According to one embodiment, the antigen is a superbug antigen (e.g. multi-drug resistant bacteria). Examples of superbugs include, but are not limited to, Enterococcus faecium, Clostridium difficile, Acinetobacter baumannii, Pseudomonas aeruginosa, and Enterobacteriaceae (including Escherichia coli, Klebsiella pneumoniae, Enterobacter spp.).

According to one embodiment, the antigen is a fungal antigen. Examples of fungi include, but are not limited to, candida, coccidiodes, cryptococcus, histoplasma, leishmania, plasmodium, protozoa, parasites, schistosomae, tinea, toxoplasma, and Trypanosoma cruzi.

As further particular examples of fungal antigens, coccidiodes antigens include, but are not limited to, spherule antigens; cryptococcal antigens include, but are not limited to, capsular polysaccharides; histoplasma antigens include, but are not limited to, heat shock protein 60 (HSP60); leishmania antigens include, but are not limited to, gp63 and lipophosphoglycan; Plasmodium falciparum antigens include, but are not limited to, merozoite surface antigens, sporozoite surface antigens, circumsporozoite antigens, gametocyte/gamete surface antigens, protozoal and other parasitic antigens including the blood-stage antigen pf 155/RESA; schistosomae antigens include, but are not limited to, glutathione-S-transferase and paramyosin; tinea fungal antigens include, but are not limited to, trichophytin; toxoplasma antigens include, but are not limited to, SAG-1 and p30; and Trypanosoma cruzi antigens include, but are not limited to, the 75-77 kDa antigen and the 56 kDa antigen.

According to one embodiment, the antigen or antigens comprise antigens associated with a malignant disease (e.g. tumor antigens).

According to one embodiment, the antigen is an antigen (or part thereof, e.g. antigen epitope) expressed by tumor cells. According to one embodiment, the antigen (or part thereof) is derived from a protein expressed in a hematopoietic tissue (e.g. hematopoietic malignancy such as leukemia antigen) or expressed in a solid tumor (e.g. melanoma, pancreatic cancer, liver cancer, gastrointestinal cancer, etc.).

Examples of tumor antigens include, but are not limited to, A33, BAGE, Bcl-2, B cell maturation antigen (BCMA), BCR-ABL, β-catenin, cancer testis antigens (CTA e.g. MAGE-1, MAGE-A2/A3 and NY-ESO-1), CA 125, CA 19-9, CA 50, CA 27.29 (BR 27.29), CA 15-3, CD5, CD19, CD20, CD21, CD22, CD33, CD37, CD45, CD123, CEA, c-Met, CS-1, cyclin B1, DAGE, EBNA, EGFR, ELA2, ephrinB2, estrogen receptor, FAP, ferritin, folate-binding protein, GAGE, G250/CA IX, GD-2, GM2, gp75, gp100 (Pmel 17), HA-1, HA-2, HER-2/neu, HM1.24, HPV E6, HPV E7, hTERT, Ki-67, LRP, mesothelin, mucin-like cancer-associated antigen (MCA), MUC1, p53, PR1, PRAME, PRTN3, RHAMM (CD168), WT-1. Further tumor antigens are provided in Molldrem J. Biology of Blood and Marrow Transplantation (2006) 12:13-18; Alatrash G. and Molldrem J., Expert Rev Hematol. (2011) 4(1): 37-50; Renkvist et al., Cancer Immunol Immunother (2001) 50:3-15; van der Bruggen P, Stroobant V, Vigneron N, Van den Eynde B. Peptide database: T cell-defined tumor antigens. Cancer Immun (2013), www(dot)cancerimmunity(dot)org/peptide/; Rittenhouse, Manderino, and Hass, Laboratory Medicine (1985) 16(9) 556-560; all of which are incorporated herein by reference.

According to one embodiment, the antigen or antigens comprise a mixture of antigens (e.g. a mixture of antigens of one group of antigens as discussed, e.g. viral antigens; or a mixture of antigens from different groups of antigens, e.g. viral and bacterial antigens, viral and tumor antigens).

According to one embodiment, the antigen or antigens comprise a mixture of viral peptides and tumor peptides (e.g. in a single formulation or in several formulations).

According to one embodiment, the antigen or antigens comprise a mixture of viral peptides and bacterial peptides (e.g. in a single formulation or in several formulations).

According to one embodiment, the antigen or antigens comprise a mixture of viral peptides and fungal peptides (e.g. in a single formulation or in several formulations).

According to one embodiment, loading of antigen presenting cells (e.g. mDCs) with an antigen or antigens can be carried out using any method known in the art.

According to one embodiment, in order to load (e.g. present) the viral peptides on antigen presenting cells (e.g. mDCs), the viral peptides are co-cultured with the antigen presenting cells (e.g. mDCs) for 30 minutes to 3 hours (e.g. 1 hour) at 37° C. at 5% CO₂. For instance, antigen presenting cells (e.g. mDCs) may be loaded with peptivators (e.g. AdV5 Hexon, HCMV pp65, EBV select and BKV LT) by incubation for 30 minutes to 3 hours (e.g. 1 hour) at 37° C. at 5% CO₂.

Following incubation, the viral peptide loaded antigen presenting cells (e.g. mDCs) are washed and centrifuged with e.g. ACD-A with 0.5% of 25% HAS and DPBS Buffer, and are resuspended in cell growth medium (e.g. T cell growth medium).

According to one embodiment, the antigen or antigens (e.g. viral peptide) loaded antigen presenting cells (e.g. mDCs) are irradiated via X-Ray source. Thus, according to one embodiment, the loaded antigen presenting cells (e.g. mDCs) are irradiated with about 1-5 Gy, about 5-10 Gy, about 10-20 Gy, about 10-30 Gy, about 10-40 Gy, about 10-50 Gy, about 20-30 Gy, about 20-40 Gy, about 20-50 Gy. According to a specific embodiment, the DCs are irradiated with about 10-40 Gy (e.g. 25-30 Gy e.g. 30 Gy).

Once irradiation is complete the loaded antigen presenting cells (e.g. viral peptide loaded mDCs are washed, centrifuged and resuspended in cell growth medium (e.g. T cell growth medium).

The antigen-loaded antigen presenting cells (e.g. mDCs) are then ready to use for generation of Tcm cells from the population of cells comprising memory CD8 T cells according to some embodiments of the invention.

According to a specific embodiment, the antigen presenting cells comprise dendritic cells (DCs).

According to a specific embodiment, the antigen presenting cells comprise mature dendritic cells (mDC).

According to a specific embodiment, at least 50%, 60%, 70%, 80%, 90%, 95%, 99% or 100% of the antigen presenting cells comprise mature dendritic cells (mDC).

According to a specific embodiment, the antigen presenting cells comprise irradiated dendritic cells.

According to one embodiment, the antigen presenting cells are of the same donor subject as the population of cells (as discussed below).

It will be appreciated that antigen presenting cells may express all of the antigens on a single cell or may express only part of the antigens on a single cell. Moreover, different antigen presenting cells (e.g. in the same preparation) may express different antigens. Accordingly, the antigen presenting cells (e.g. mDC) comprise a heterogeneous cell mixture.

According to some embodiments of the invention, the antigen or antigens (e.g. viral peptides) can be presented by genetically modified antigen presenting cells or artificial antigen presenting cells exhibiting MHC antigens (also referred to as human leukocyte antigen (HLA)) recognizable by the memory CD8 T cells (e.g. cell line transfected with the antigen or antigens). Additionally or alternatively, antigen or antigens (e.g. viral peptides) of some embodiments of the invention can be displayed on an artificial vehicle (e.g. liposome).

Thus, antigen presenting cells (as discussed above), cell lines, artificial vehicles (such as a liposome) or artificial antigen presenting cells (e.g. leukemic or fibroblast cell line transfected with the antigen or antigens), can be used to present short synthetic peptides fused or loaded thereto or to present protein extracts or purified proteins. Such short peptides, protein extracts or purified proteins may be viral-, bacterial-, fungal-, or tumor-antigen derived peptides or peptides representing any other antigen.

As mentioned above, the method of some embodiments of the invention is affected by providing a population of cells comprising T cells, wherein the T cells in the population of cells comprise at least 40% memory T cells expressing a CD45RA⁻CD8⁺ phenotype and depleted of CD4⁺, CD56⁺ and CD45RA⁺ expressing cells.

The term “population of cells comprising T cells” refers to a heterogeneous mixture of PBMCs comprising T cells, B cells and myeloid cells. The population of cells comprising T cells typically comprises T cells having numerous signatures, functions and capable of binding various antigens (e.g. cytotoxic T cells, memory T cells, effector T cells etc.).

According to one embodiment, the population of cells comprising T cells does not comprise erythrocytes and granulocytes.

The term “memory T cells” as used herein refers to a subset of T lymphocytes which have previously encountered and responded to an antigen, also referred to as antigen experienced T cells.

According to one embodiment, the memory T cells comprise at least about 20%, at least about 30%, at least about 40%, at least about 50%, at least about 60%, at least about 70%, at least about 80%, at least about 90%, at least about 95%, at least about 99%, or even 100% of the T cells in the population of cells.

It will be appreciated that under normal conditions (i.e. as determined in a healthy subject), the level of memory T cells comprises less than 20% of the total number of cells in a population of cells comprising T cells.

According to one embodiment, the memory T cells comprise T cells expressing a CD8 marker (i.e. CD8⁺ T cells).

According to another embodiment, the memory T cells comprise a CD8⁺CD45RO′ phenotype.

According to another embodiment, the memory T cells comprise a CD8⁺CD45RA-phenotype.

According to another embodiment, the memory T cells comprise a CD8⁺CD45RO⁺CD45RA-phenotype.

According to one embodiment, the memory T cells are devoid of CD45RA⁺ cells.

According to one embodiment, the memory T cells are devoid of CD4⁺ and/or CD56⁺ cells.

Selection of memory CD8⁺ T cells may be affected by selection of cells co-expressing CD8⁺ and CD45RA⁻ and/or cells co-expressing CD8⁺ and CD45RO′ and may be carried out using any method known in the art, such as by affinity based purification (e.g. such as by the use of MACS beads, FACS sorter and/or capture ELISA labeling).

Selection of memory CD8⁺ T cells may be further affected by selection of effector T cells and central memory T cells, the latter expressing e.g. CD62L, CCR7, CD27 and/or CD28.

In order to obtain a population of cells in which the T cell population comprises a high purity of memory T cells (e.g. at least about 30-50% memory T cells, e.g. 40% memory T cells) or in order to increase the number of memory T cells, PBMCs may be depleted of naive cells, e.g. CD45RA⁺ cells, of CD4⁺ cells (e.g. T helper cells), of CD56⁺ cells (e.g. NK cells) or any other cells not comprising a memory T cell phenotype.

Depletion of naïve T cells (e.g. expressing CD45RA⁺ cells), CD4⁺ and/or CD56+ cells may be carried out using any method known in the art, such as by affinity based purification (e.g. such as by the use of MACS beads, FACS sorter and/or capture ELISA labeling).

According to one embodiment, memory T cells are obtained from peripheral blood mononuclear cells (PBMCs).

According to one embodiment, memory T cells are obtained by a method comprising treating a second population of PBMCs of the same donor subject as the first population of PBMCs with one or more agents capable of depleting CD4⁺, CD56⁺ and CD45RA⁺ expressing cells so as to obtain a population of cells which include at least 40% memory T cells expressing a CD45RA⁻CD8⁺ phenotype.

According to one embodiment, the population of cells further comprises B cells and myeloid cells.

According to one embodiment, the CD14⁻ cells collected from the first population of cells are combined with the second population of cells prior to enrichment of memory T cells.

According to one embodiment, prior to enrichment of memory T cells, the CD14⁻ cells obtained from the first population of cells and/or the PBMCs obtained from the second population of cells are centrifuged and resuspended at a concentration of e.g. 10-50×10⁶ cells/ml, e.g. 30×10⁶ cells/ml, in cell growth media e.g. T Cell Growth Media (e.g. Click's Media with advanced RPMI 1640 supplemented with 1:100 Glutamaxe and 5% Human AB Serum) along with IL-7 (30 IU/mL)). According to one embodiment, the cell growth media is supplemented with IL-7 (e.g. at a concentration of e.g. 1-100 IU/mL, e.g. 30 IU/mL). The cell suspension is then seeded (e.g. in tissue culture flasks) and incubated for 12-36 hours, e.g. for 16-24 hours, e.g. for 24 hours, in at 37° C., 5% CO₂.

According to one embodiment, the second population of PBMCs are centrifuged and resuspended in buffer (e.g. in CliniMACS®/0.5% HSA Buffer) to a minimum of 1:2 ratio.

According to one embodiment, the second population of PBMCs are platelet washed (e.g. thrombowash), centrifuged and resuspended in in buffer (e.g. CliniMACS®/0.5% HSA buffer).

According to one embodiment, the post-platelet depleted cell preparation of one embodiment is incubated with IVIg for 5-30 minutes e.g. 10-15 minutes. After the initial incubation the CD4⁺, CD56⁺ and CD45RA⁺ binding agents are added to the cell preparation and incubated for e.g. 10-60 minutes, e.g. 30 minutes, on an orbital rotator.

According to one embodiment, the CD4⁺, CD56⁺ and/or CD45RA⁺ binding agent is an antibody.

According to a specific embodiment, the CD4⁺, CD56⁺ and/or CD45RA⁺ binding agent is a monoclonal antibody.

According to a specific embodiment, the CD4⁺, CD56⁺ and/or CD45RA⁺ monoclonal antibody is conjugated to magnetic particles.

According to a specific embodiment, the CD4⁺, CD56⁺ and/or CD45RA⁺ monoclonal antibody is conjugated to super-paramagnetic particles.

According to one embodiment, at the end of the incubation, the cells are washed by centrifugation and the cell pellet resuspended in buffer (e.g. CliniMACS®/0.5% HSA buffer) to remove excess reagent.

According to one embodiment, the CD4⁺/CD56⁺/CD45RA⁺ labeled cells are selected by magnetic separation techniques (as discussed in detail hereinabove).

According to a specific embodiment, the CD4⁺/CD56⁺/CD45RA⁺ labeled cells are processed on CliniMACS® column.

According to one embodiment, the CD4⁺/CD56⁺/CD45RA⁺ magnetically labeled cells (i.e. CD4⁺/CD56⁺/CD45RA⁺ expressing cells) are retained by the separation column (i.e. negative selection) and the CD4⁻/CD56⁻/CD45RA⁻ cells are collected. According to one embodiment, the collected cells are washed and resuspended in T cell Growth Medium. According to one embodiment, samples from each fraction are removed for cell count, viability and/or immunophenotyping.

According to one embodiment, the collected CD4⁻/CD56⁻/CD45RA⁻ cell fraction is adjusted at 0.01-10×10⁶ cells/ml, e.g. 2×10⁶ cells/ml, in T Cell Growth Media supplemented with cytokines and growth factors. Determination of cytokines and growth factors to be used is within the skill of a person of skill in the art. For example, the T Cell Growth Media is supplemented with IL-7 (e.g. at a concentration of e.g. 1-100 IU/mL, e.g. 30 IU/mL). The cell suspension is then seeded (e.g. in G-Rex®100) and incubated for 12-36 hours, e.g. for 16-24 hours, e.g. for 24 hours, in at 37° C., 5% CO₂.

In order to deplete alloreactive clones from the memory T cell pool by way of antigen (e.g. viral antigen) activation, the memory T cells are contacted with the antigenic peptides, e.g. viral peptides.

Typically, the non-GVHD inducing cells comprising a Tcm phenotype of the present invention are generated by contacting a population of cells comprising memory T cells with antigen presenting cells loaded with the antigenic peptides e.g. viral peptides (such as described above) in a culture supplemented with IL-21 (e.g. in an otherwise cytokine-free culture i.e., without the addition of any additional cytokines). This step is typically carried out for about 12-24 hours, about 12-36 hours, about 12-72 hours, 12-96 hours, 12-120 hours, about 24-36 hours, about 24-48 hours, about 24-72 hours, about 36-48 hours, about 36-72 hours, about 48-72 hours, about 48-96 hours, about 48-120 hours, 0.5-1 days, 0.5-2 days, 0.5-3 days, 0.5-5 days, 1-2 days, 1-3 days, 1-5 days, 1-7 days, 1-10 days, 2-3 days, 2-4 days, 2-5 days, 2-6 days, 2-8 days, 3-4 days, 3-5 days, 3-7 days, 4-5 days, 4-8 days, 5-7 days, 6-8 days or 8-10 days and allows enrichment of antigen (e.g. viral antigen) reactive cells.

According to a specific embodiment, contacting a population of PBMC depleted of CD4+, CD56+ and CD45RA+ cells and comprising memory CD8⁺ T cells (e.g. a population of cells comprising T cells, wherein the T cells in the population of cells comprise at least 40% memory T cells), with an antigen or antigens (such as described above) in a culture supplemented with IL-21 (otherwise cytokine-free culture) is affected for 12 hours-6 days (e.g. 3 days).

Contacting a population of cells comprising memory CD8⁺ T cells with an antigen or antigens (such as described above) in a culture supplemented with IL-21 is typically carried out in the presence of about 0.001-3000 IU/ml, 0.01-3000 IU/ml, 0.1-3000 IU/ml, 1-3000 IU/ml, 10-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 250-1000 IU/ml, 500-1000 IU/ml, 750-1000 IU/ml, 10-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-21. According to a specific embodiment, the concentration of IL-21 is 50-500 IU/ml (e.g. 100 IU/ml).

According to a specific embodiment, contacting a population of cells comprising memory CD8⁺ T cells with an antigen or antigens is affected in a cytokine-free culture (e.g. supplemented with only IL-21), such a culture condition enables survival and enrichment of only those cells which undergo stimulation and activation by the antigen or antigens (i.e. of antigen reactive cells, e.g. viral reactive memory T cells) as these cells secrete cytokines (e.g. IL-2) which enable their survival (all the rest of the cells die under these culture conditions).

According to one embodiment, the ratio of the population of cells comprising memory CD8⁺ T cells (i.e. CD4⁻CD56⁻CD45RA⁻ cells) to antigenic (e.g. viral) peptide loaded antigen presenting cells (e.g. mDCs) is typically about 2:1 to about 10:1, such as about 2:1, about 3:1, about 4:1, about 5:1, about 6:1, about 8:1 or about 10:1. According to a specific embodiment, the ratio of the population of cells comprising memory CD8⁺ T cells (i.e. CD4⁻CD56⁻CD45RA⁻ cells) to antigenic (e.g. viral) peptide loaded antigen presenting cells (e.g. mDCs) is about 2:1 to 8:1, e.g. about 5:1.

According to a specific embodiment, the population of cells comprising memory CD8⁺ T cells (i.e. CD4⁻CD56⁻CD45RA⁻ cells) are seeded (e.g. in G-Rex®100) at a concentration of 0.01-10×10⁶ cells/ml, e.g. 1×10⁶ cells/ml, together with the viral peptide loaded antigen presenting cells (e.g. mDCs) at a ratio of about 2:1 to about 8:1, e.g. about 5:1 (memory CD8⁺ T cells: antigen presenting cells (e.g. mDC)) in T Cell Growth Media along with IL-21 (e.g. at a concentration of 50-500 IU/ml, e.g. 100 IU/ml) for 1-5 days (e.g. 3 days) in 37° C., 5% CO₂.

Next, the resultant population of cells comprising memory CD8⁺ T cells (i.e. after culture with IL-21) are cultured in the presence of IL-21, IL-15 and/or IL-7 so as to allow proliferation of cells comprising the Tcm phenotype. This step is typically carried out for about 12-24 hours, about 12-36 hours, about 12-72 hours, about 12-96 hours, about 12-120 hours, about 12-240 hours, 24-36 hours, 24-48 hours, about 24-72 hours, 24-96 hours, 24-120 hours, 24-240 hours, about 48-72 hours, about 48-120 hours, about 48-240 hours, about 96-240 hours, about 120-144 hours, about 120-240 hours, about 144-240 hours, 0.5-1 days, 0.5-2 days, 0.5-3 days, 0.5-5 days, 0.5-10 days, 1-2 days, 1-3 days, 1-4 days, 1-6 days, 1-8 days, 1-9 days, 1-10 days, 2-3 days, 2-4 days, 2-5 days, 2-6 days, 2-8 days, 2-9 days, 2-10 days, 4-5 days, 4-6 days, 4-8 days, 4-9 days, 4-10 days, 5-6 days, 5-7 days, 5-8 days, 5-9 days, 5-10 days, 5-15 days, 6-7 days, 6-8 days, 6-9 days, 6-10 days, 6-12 days, 7-8 days, 7-9 days, 7-11 days, 7-13 days, 7-15 days, 8-9 days, 8-10 days, 9-10 days, 9-12 days, 9-15 days, 10-12 days, 10-15 days, 12-15 days, 14-16 days, 14-18 days, 16-18 days or 18-20 days. According to a specific embodiment, the resultant population of cells comprising memory CD8⁺ T cells (i.e. after culture with IL-21) are cultured in the presence of IL-21, IL-15 and IL-7 for about 6-12 days (e.g. 9 days).

This step is typically carried out in the presence of IL-21 at a concentration of about 0.001-3000 IU/ml, 0.01-3000 IU/ml, 0.1-3000 IU/ml, 1-3000 IU/ml, 10-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 250-1000 IU/ml, 500-1000 IU/ml, 750-1000 IU/ml, 10-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-21. According to a specific embodiment, the concentration of IL-21 is 50-500 IU/ml (e.g. 100 IU/ml).

This step is further carried out in the presence of IL-15 at a concentration of about 0.001-3000 IU/ml, 0.01-3000 IU/ml, 0.1-3000 IU/ml, 1-3000 IU/ml, 10-3000 IU/ml, 100-3000 IU/ml, 125-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 125-1000 IU/ml, 250-1000 IU/ml, 500-1000 IU/ml, 750-1000 IU/ml, 10-500 IU/ml, 50-500 IU/ml, 100-500 IU/ml, 125-500 IU/ml, 250-500 IU/ml, 250-500 IU/ml, 125-250 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-15. According to a specific embodiment the concentration of IL-15 is 50-500 IU/ml (e.g. 125 IU/ml).

This step is further carried out in the presence of IL-7 at a concentration of about 0.001-3000 IU/ml, 0.01-3000 IU/ml, 0.1-3000 IU/ml, 1-3000 IU/ml, 10-3000 IU/ml, 30-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 30-1000 IU/ml, 100-1000 IU/ml, 250-1000 IU/ml, 500-1000 IU/ml, 750-1000 IU/ml, 10-500 IU/ml, 30-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-7. According to a specific embodiment the concentration of IL-7 is 1-100 IU/ml (30 IU/ml).

According to a specific embodiment, the cell culture comprising the resultant population of cells comprising memory CD8⁺ T cells (i.e. after culture with IL-21) is supplemented with IL-7 (e.g. at a concentration of e.g. 1-100 IU/ml, e.g. 30 IU/mL), IL-15 (e.g. at a concentration of e.g. 50-500 IU/ml, e.g. 125 IU/mL) and IL-21 (e.g. at a concentration of 50-500 IU/ml, e.g. 100 IU/mL) at 50% of the culture volume, and cultured for about 6-12 days (e.g. 9 days) while supplementing IL-7, IL-15, IL-21 every about 48-96 hours, e.g. 48 hours, e.g. 72 hours.

According to one embodiment, the total length of culturing time for generating the Tcm cells is about 9, 10, 11, 12, 13, 14, 15, 17, 19 or 21 days (e.g. 12 days).

According to one embodiment, the cell culture comprising the resultant population of cells comprising memory CD8⁺ T cells (i.e. after culture with IL-21) is monitored for glucose levels.

According to one embodiment, the glucose is at a level comprising 10-500 mg/dl, e.g. 50-170 mg/dl.

According to one embodiment, when the glucose level is between 170 mg/dL and 130 mg/dL, cytokines IL-7, IL-15, IL-21 are added to the culture (as detailed above).

According to one embodiment, when the glucose level is between 129 mg/dL to 100 mg/dL, fresh T cell Growth medium e.g. 25% volume, e.g. 100 ml (e.g. 25% of the G-Rex®100 volume of 400 ml) plus cytokines IL-7, IL-15, IL-21 are added to the culture.

According to one embodiment, when the glucose level is between 99 mg/dL to 50 mg/dL, fresh T cell Growth medium e.g. 50% volume, e.g. 200 ml (e.g. 50% of the G-Rex®100 volume of 400 ml) plus cytokines IL-7, IL-15, IL-21 are added to the culture.

According to one embodiment, culturing further comprises adding glucose to a concentration of at least about 20 mg/dl, at least about 30 mg/dl, at least about 40 mg/dl, at least about 50 mg/dl, at least about 60 mg/dl, at least about 70 mg/dl, at least about 80 mg/dl, at least about 90 mg/dl, at least about 100 mg/dl

According to a specific embodiment, culturing further comprises adding glucose to a concentration of at least about 50 mg/dl.

According to one embodiment, the cell culture comprising the resultant population of cells comprising memory CD8⁺ T cells (i.e. after culture with IL-21) is monitored for pH levels. According to one embodiment, the pH is at the physiologic range (e.g. pH 7.2-7.6). In case the pH level is not at a physiological range, the pH level may be adjusted using any method known in the art.

According to one embodiment of the invention, there is provided a method of generating an isolated population of non-graft versus host disease (GVHD) inducing cells comprising a central memory T-lymphocyte (Tcm) phenotype, the cells being tolerance inducing cells and/or endowed with anti-disease activity, and capable of homing to the lymph nodes following transplantation, the method comprising:
(a) contacting a first population of peripheral blood mononuclear cells (PBMCs) from a donor subject with an antibody capable of selecting CD14⁺ expressing cells and selecting CD14⁺ expressing cells capable of maturing into dendritic cells;
(b) loading dendritic cells with viral peptides derived from 4-10 types of viruses, wherein at least one of the viruses comprises a BK virus;
(c) treating a second population of PBMCs of the same donor subject as the first population of PBMCs with one or more agents capable of depleting CD4⁺, CD56⁺ and CD45RA⁺ expressing cells so as to obtain a population of cells comprising memory T cells expressing a CD45RA⁻CD8⁺ phenotype;
(d) contacting the population of cells comprising the memory T cells with the dendritic cells loaded with the viral peptides of step (b) in the presence of IL-21 so as to allow enrichment of viral reactive memory T cells; and
(e) culturing the cells resulting from step (d) in the presence of IL-21, IL-15 and/or IL-7 so as to allow proliferation of cells comprising the Tcm phenotype, thereby generating the isolated population of non-GVHD inducing cells comprising the Tcm phenotype.

According to one embodiment, culturing the resultant population of cells comprising memory CD8⁺ T cells (i.e. after culture with IL-21) in the presence of IL-21, IL-15 and/or IL-7 is typically affected in an antigen free environment (i.e. without the addition of an antigen or antigens, e.g. viral peptides). However, it is to be understood that residual antigen or antigens (e.g. viral peptides) can be present in the cell culture after culture with IL-21 (i.e. in the Tcm proliferation step comprising, for example, the addition of IL-21, IL-15 and IL-7) and thus an antigen free environment relates to a cell culture without the addition of supplementary antigen presenting cells presenting antigen or antigens (e.g. viral peptides).

According to one embodiment, in order to obtain memory CD8⁺ T cells specific to an antigen or antigens, the antigen/s (e.g. tumor antigen, viral antigen) is administered to the donor subject prior to obtaining memory CD8⁺ T cells therefrom (e.g. prior to providing the population of T cells comprising at least 40% memory CD8⁺ T cells). Any method of immunizing a cell donor against an antigen in order to elicit an immunogenic response (e.g. generation of memory CD8⁺ T cells) may be employed.

The antigen may be administered as is or as part of a composition comprising an adjuvant (e.g. Complete Freund's adjuvant (CFA) or Incomplete Freund's adjuvant (IFA)). According to one embodiment, the antigen is administered to a donor subject once. According to one embodiment, the donor subject receives at least one additional (e.g. boost) administration of the antigen (e.g. 2, 3, 4 or more administrations). Such an additional administration may be affected 1, 3, 5, 7, 10, 12, 14, 21, 30 days or more following the first administration of the antigen.

In order to further enrich the memory CD8⁺ T cells against a particular antigen/s and to deplete alloreactive clones from the memory T cell pool, the population of cells comprising memory CD8⁺ T cells may be further contacted with the same antigen or antigens (e.g. the same antigen as administered to the cell donor), as described hereinabove.

It will be appreciated that cell samples and culture medium samples can be obtained at any stage during the process of generating the isolate population of non-GVHD inducing cells comprising the Tcm phenotype. These can be used for evaluating cell count, cell viability, sterility, immunophenotyping, glucose and pH levels, etc. Any method known in the art can be used to implement such procedures.

According to one embodiment, there is provided an isolated population of non-GVHD inducing cells comprising cells having a central memory T-lymphocyte (Tcm) phenotype, the cells being tolerance inducing cells and/or endowed with anti-disease activity, and capable of homing to the lymph nodes following transplantation, generated according to the method of some embodiments of the invention.

Following generation of the non-GVHD inducing comprising the Tcm phenotype, the cells may be used as fresh cells (e.g. within about 1, 2, 3, 4, 5, 6, 7, 8, 9 or 10 days, e.g. within about 3 days).

Alternatively, the cells may be cryopreserved until needed (e.g. for 1 week, 2 weeks, 1 month, 2 months, 4 months, 6 months, a year or more).

According to one embodiment, the non-GVHD inducing cells comprising the Tcm phenotype of the invention are not naturally occurring and are not a product of nature. These cells are typically produced by ex-vivo manipulation (i.e. exposure to an antigen or antigens in the presence of specific cytokines).

The above describe protocols are typically used for non-syngeneic applications and therefore the population of cells comprising memory T cells or PBMC used are typically allogeneic with respect to a recipient subject (e.g. from an allogeneic donor). Likewise, in cases in which xenogeneic applications may be beneficial, the memory T cells or PBMC used may be of a xenogeneic origin as discussed below. However, in cases in which a syngeneic applications may be beneficial, the population of cells comprising memory T cells or PBMC used may be autologous with respect to a recipient subject (e.g. from the subject). Such determinations are well within the capability of one of skill in the art, especially in view of the disclosure provided.

Thus, as mentioned, the population of cells comprising memory CD8⁺ T cells or PBMC may be syngeneic or non-syngeneic with respect to a subject.

As used herein, the term “syngeneic” cells refer to cells which are essentially genetically identical with the subject or essentially all lymphocytes of the subject. Examples of syngeneic cells include cells derived from the subject (also referred to in the art as an “autologous”), from a clone of the subject, or from an identical twin of the subject.

As used herein, the term “non-syngeneic” cells refer to cells which are not essentially genetically identical with the subject or essentially all lymphocytes of the subject, such as allogeneic cells or xenogeneic cells.

As used herein, the term “allogeneic” refers to cells which are derived from a donor subject who is of the same species as the recipient subject, but which is substantially non-clonal with the recipient subject. Typically, outbred, non-zygotic twin mammals of the same species are allogeneic with each other. It will be appreciated that an allogeneic cell may be HLA identical, partially HLA identical or HLA non-identical (i.e. displaying one or more disparate HLA determinant) with respect to the recipient subject.

According to one embodiment, the donor is a human being.

As used herein, the term “xenogeneic” refers to a cell which substantially expresses antigens of a different species relative to the species of a substantial proportion of the lymphocytes of the subject. Typically, outbred mammals of different species are xenogeneic with each other.

The present invention envisages that xenogeneic cells are derived from a variety of species. Thus, according to one embodiment, the cells may be derived from any mammal. Suitable species origins for the cells comprise the major domesticated or livestock animals and primates. Such animals include, but are not limited to, porcines (e.g. pig), bovines (e.g., cow), equines (e.g., horse), ovines (e.g., goat, sheep), felines (e.g., Felis domestica), canines (e.g., Canis domestica), rodents (e.g., mouse, rat, rabbit, guinea pig, gerbil, hamster), and primates (e.g., chimpanzee, rhesus monkey, macaque monkey, marmoset). Cells of xenogeneic origin (e.g. porcine origin) are preferably obtained from a source which is known to be free of zoonoses, such as porcine endogenous retroviruses. Similarly, human-derived cells or tissues are preferably obtained from substantially pathogen-free sources.

Thus, the source of the population of cells comprising memory CD8⁺ T cells or PBMCs will be determined with respect to the intended use of the cells (see further details hereinbelow) and is well within the capability of one skilled in the art, especially in light of the detailed disclosure provided herein.

The use of veto cells is especially beneficial in situations in which there is a need to eliminate graft rejection, overcome graft versus host disease (GVHD) and/or to induce donor specific tolerance, such as in transplantation of allogeneic or xenogeneic cells or tissues. The use of veto cells can also be beneficial for prolongation of “off-the-shelf” allogenic genetically modified T cells such as CAR-T cells or TCR transgenic T cells (TCR-T).

Accordingly, the veto cells of some embodiments of the invention may be used as an adjuvant therapy to facilitate engraftment of genetically modified cells transduced to express a cell surface receptor comprising a T cell receptor signaling module (i.e. an intracellular portion of the receptor responsible for activation of at least one of the normal effector functions of the T cell in which the receptor has been placed in), e.g. a transgenic T cell receptor (tg-TCR) or a chimeric antigen receptor (CAR), as discussed in detail herein above.

According to one embodiment, the veto cells of some embodiments of the invention can be used in conjunction with any CAR-T or TCR-T cells (e.g. CAR-T or TCR-T cells generated from cells of the veto cell donor), such as tumor specific CAR-T or TCR-T cells targeting a variety of tumor antigens for the treatment of hematopoietic or solid cancers. Examples of these cancers and their antigens that can be targeted include, but are not limited to, follicular lymphoma (CD20 or GD2), neuroblastoma (CD171), non-Hodgkin lymphoma (CD20), lymphoma (CD19), glioblastoma (IL13Rα2), chronic lymphocytic leukemia or CLL, Multiple Myeloma (BCMA, CD138, CS1) and acute lymphocytic leukemia or ALL (both CD19).

It will be appreciated that the veto cells and the CAR-T/TCR-T cells can be used concomitantly or subsequent to each other (e.g. on the same day or within e.g. about 1, 2, 3, 4, 5, 6, 7 days of each other).

As mentioned above, the veto cells of the invention are further endowed with anti-disease activity (e.g. anti-viral activity or anti-tumor activity e.g. GVL) and are therefore beneficial in situations in which a subject, e.g. transplanted subject, has a disease or condition (e.g. malignant, viral, bacterial, fungal, autoimmune or allergic disease or condition), pre- or post-transplantation (e.g. before immune reconstitution is established).

According to one embodiment, the veto cells of the invention are beneficial for preventing viral infection.

According to one embodiment, the veto cells of the invention are beneficial for killing cancer cells including residual cancer cells (e.g. leukemic cells).

According to one embodiment, the veto cells of the invention are beneficial for preventing disease relapse (e.g. leukemia relapse).

Thus, according to another aspect of the present invention, there is provided a method of treating a disease in a subject in need thereof, the method comprising administering to the subject a therapeutically effective amount of the isolated population of non-GVHD inducing cells of some embodiments of the invention (i.e. Tcm cells), thereby treating the disease in the subject.

According to another aspect of the invention, there is provided a use of the non-GVHD inducing cells comprising a Tcm phenotype of some embodiments of the invention for the manufacture of a medicament identified for treating a disease in a subject in need thereof.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “subject” or “subject in need thereof” refers to a mammal, preferably a human being, male or female at any age that is in need of a cell or tissue transplantation or suffers from a disease which may be treated with the non-GVHD inducing Tcm cells. Typically the subject is in need of cell or tissue transplantation (also referred to herein as recipient) due to a disorder or a pathological or undesired condition, state, or syndrome, or a physical, morphological or physiological abnormality which is amenable to treatment via cell or tissue transplantation. Examples of such disorders are provided further below.

Thus, the method of the present invention may be applied to treat any disease such as, but not limited to, a malignant disease, a disease associated with transplantation of a graft (e.g. graft rejection, graft versus host disease), an infectious disease (e.g. viral disease, fungal disease or a bacterial disease), an inflammatory disease, an autoimmune disease and/or an allergic disease or condition.

According to one embodiment, the subject has a malignant disease.

Malignant diseases (also termed cancers) which can be treated by the method of some embodiments of the invention can be any solid or non-solid tumor and/or tumor metastasis and/or disease relapse.

Examples of cancer include, but are not limited to, carcinoma, lymphoma, blastoma, sarcoma, and leukemia. More particular examples of such cancers include squamous cell cancer, soft-tissue sarcoma, Kaposi's sarcoma, melanoma, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, rectal cancer, endometrial or uterine carcinoma, carcinoid carcinoma, salivary gland carcinoma, kidney or renal cancer, liver cancer, prostate cancer, vulval cancer, thyroid cancer, hepatic carcinoma, mesothelioma, multiple myeloma, post-transplant lymphoproliferative disorder (PTLD), and various types of head and neck cancer (e.g. brain tumor). The cancerous conditions amenable for treatment of the invention include metastatic cancers.

According to a specific embodiment, the malignant disease is a leukemia, a lymphoma, a myeloma, a melanoma, a sarcoma, a neuroblastoma, a colon cancer, a colorectal cancer, a breast cancer, an ovarian cancer, an esophageal cancer, a synovial cell cancer, a hepatic cancer and a pancreatic cancer.

According to one embodiment, the malignant disease is a hematological malignancy. Exemplary hematological malignancies include, but are not limited to, leukemia [e.g., acute lymphatic, acute lymphoblastic, acute lymphoblastic pre-B cell, acute lymphoblastic T cell leukemia, acute—megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia, T-cell acute lymphocytic leukemia (T-ALL) and B-cell chronic lymphocytic leukemia (B-CLL)] and lymphoma [e.g., Hodgkin's disease, non-Hodgkin's lymphoma, Burkitt, cutaneous T cell, histiocytic, lymphoblastic, T cell, thymic, B cell, including low grade/follicular; small lymphocytic (SL) NHL; intermediate grade/follicular NHL; intermediate grade diffuse NHL; high grade immunoblastic NHL; high grade lymphoblastic NHL; high-grade small non-cleaved cell NHL; bulky disease NHL; mantle cell lymphoma; AIDS-related lymphoma; and Waldenstrom's Macroglobulinemia].

According to a specific embodiment, the hematological malignancy is a follicular lymphoma (FL), mantle cell lymphoma (MCL), chronic lymphocytic leukemia (CLL), multiple myeloma (MM), Hodgkin's lymphoma (HL), non-hodgkin's lymphoma (NHL), chronic myeloid leukemia (CML), myeloproliferative syndromes (MPD), acute myeloid leukemia (AML) or acute lymphoid leukemia (ALL).

According to one embodiment, the subject has a non-malignant disease.

According to one embodiment, the non-malignant disease is an organ dysfunction or failure, a hematologic disease, a graft related disease, an infectious disease, an inflammatory disease, an autoimmune disease, an allergic disease, a genetic disease or disorder, a metabolic disorder, a trauma and an injury.

According to one embodiment the subject of the present invention may suffer from any of a cardiovascular disease, a rheumatoid disease, a glandular disease, a gastrointestinal disease, a cutaneous disease, a hepatic disease, a neurological disease, a muscular disease, a nephric disease, a connective tissue disease, a systemic disease and/or a disease related to reproduction, treatable by cell or tissue transplantation.

Exemplary non-malignant diseases include, but are not limited to, severe combined immunodeficiency syndromes (SCID), sickle cell disease (e.g. sickle cell anemia), congenital neutropenia, thrombocytopenia, aplastic anemia (e.g. severe aplastic anemia), myelodysplastic syndrome, monosomy 7, osteopetrosis, Gaucher's disease, Hurler's disease, metachromatic leukodystrophy, adrenal leukodystrophy, thalassemia, congenital or genetically-determined hematopoietic abnormality, adenosine deaminase (ADA), lupus, autoimmune hepatitis, celiac disease, type I diabetes mellitus, Grave's disease, Guillain-Barr syndrome, Myasthenia gravis, Rheumatoid arthritis, scleroderma and psoriasis.

According to one embodiment, the non-malignant disease may be treated by transplantation of hematopoietic progenitor cells assisted by the addition of veto cells.

According to one embodiment, the non-malignant disease may be treated by transplantation of a cell or tissue graft (as discussed below), assisted by transplantation of hematopoietic progenitor cells and veto cells (e.g. inducing donor chimerism).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Specific types of viral pathogens causing infectious diseases treatable according to the teachings of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to the teachings of the present invention include, but are not limited to, those caused by human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, Adenovirus (Adv), cold viruses, flu viruses, Japanese encephalitis, polio, respiratory syncytial, rubella, smallpox, varicella zoster, rotavirus, West Nile virus and zika virus.

Specific examples of bacterial infections which may be treated according to the teachings of the present invention include, but are not limited to, those caused by anthrax; gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus and tetanus.

Specific examples of superbug infections (e.g. multi-drug resistant bacteria) which may be treated according to the teachings of the present invention include, but are not limited to, those caused by Enterococcus faecium, Clostridium difficile, Acinetobacter baumannii, Pseudomonas aeruginosa, and Enterobacteriaceae (including Escherichia coli, Klebsiella pneumoniae, Enterobacter spp).

Specific examples of fungal infections which may be treated according to the teachings of the present invention include, but are not limited to, those caused by candida, coccidiodes, cryptococcus, histoplasma, leishmania, plasmodium, protozoa, parasites, schistosomae, tinea, toxoplasma, and Trypanosoma cruzi.

Graft Rejection Diseases

According to other embodiment, the disease is associated with transplantation of a graft. Examples of diseases associated with transplantation of a graft include, but are not limited to, graft rejection, chronic graft rejection, subacute graft rejection, hyperacute graft rejection, acute graft rejection, allograft rejection, xenograft rejection and graft-versus-host disease (GVHD).

Non-Malignant Hematologic Disease

Examples of Non-malignant hematologic diseases include, but are not limited to, anemia, bone marrow disorders, deep vein thrombosis/pulmonary embolism, diamond blackfan anemia, hemochromatosis, hemophilia, immune hematologic disorders, iron metabolism disorders, sickle cell disease (e.g. sickle cell anemia), thalassemia, thrombocytopenia and Von Willebrand disease.

According to a specific embodiment, the non-malignant disease is a sickle cell disease or an autoimmune disease (e.g. treatable by HCT plus veto cells, as discussed below).

Non-limiting examples of autoimmune diseases include, but are not limited to, Acute disseminated encephalomyelitis (ADEM), Addison's disease, Agammaglobulinemia, Alopecia areata, Amyotrophic lateral sclerosis, Ankylosing Spondylitis, Antiphospholipid syndrome, Antisynthetase syndrome, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune uveitis (e.g. non-infectious uveitis), Behcet's disease, Celiac disease, Chronic Glomerulonephritis, Cold agglutinin disease, Crohn's disease, Dermatomyositis, Dermatomyositis, Diabetes mellitus type 1 (Type 1 diabetes mellitus (T1DM)), Eosinophilic fasciitis, Goodpasture's syndrome, Graves' disease, Guillain-Barre syndrome (GBS), Hashimoto's encephalopathy, Hashimoto's thyroiditis, Idiopathic thrombocytopenic purpura, Immune Thrombocytic Purpura (ITP), Lupus erythematosus (SLE), Miller-Fisher syndrome, Mixed connective tissue disease, Multiple sclerosis (MS), Myasthenia gravis, Narcolepsy, Pemphigus vulgaris, Pernicious anaemia, Polymyositis Primary biliary cirrhosis, Psoriasis, Psoriatic arthritis, Relapsing polychondritis, Rheumatoid arthritis (RA), Rheumatic fever, Systemic Scleroderma, Sjogren's syndrome, Temporal arteritis, Transverse myelitis, Ulcerative colitis (UC), Undifferentiated connective tissue disease, Vasculitis, and Wegener's granulomatosis.

According to a specific embodiment, the autoimmune disease is Lupus erythematosus (SLE), Multiple sclerosis (MS) and Diabetes mellitus type 1 (Type 1 diabetes mellitus (T1DM)).

According to a specific embodiment, the non-malignant disease is an aplastic anemia, a severe immune deficiency and a non-malignant bone marrow failure.

As mentioned above, in order to enhance the anti-disease activity of the non-GVHD inducing cells comprising a Tcm phenotype, it is beneficial to select an antigen or antigens (e.g. viral antigens) associated with the disease to be treated and to generate antigen specific Tcm cells for treatment.

Additionally or alternatively, as discussed above, the non-GVHD inducing cells comprising a Tcm phenotype may be genetically modified for therapy.

As discussed above, the non-GVHD inducing cells comprising a Tcm phenotype of the invention are endowed with veto activity. Accordingly, the Tcm cells of the present invention may be used as adjuvant therapy for a cell or tissue transplant. As the non-GVHD inducing cells comprising a Tcm phenotype of the present invention are also endowed with anti-disease activity the method of the present invention can furthermore be advantageously applied towards treating a disease in a subject while concomitantly facilitating engraftment of a transplant of cells or tissues.

According to one embodiment, the method further comprises transplanting a cell or tissue transplant into a subject.

According to one embodiment, the medicament further comprises a cell or tissue transplant.

According to one embodiment, there is provided a method of treating a subject in need of a non-syngeneic cell or tissue transplant, the method comprising: (a) transplanting a cell or tissue transplant into the subject; and (b) administering to the subject a therapeutically effective amount of the isolated population of non-GVHD inducing cells of some embodiments of the invention, thereby treating the subject in need of the cell or tissue transplant.

According to one embodiment, there is provided a use of the isolated population of non-GVHD inducing cells of some embodiments of the invention for the manufacture of a medicament identified as an adjuvant treatment for a non-syngeneic cell or tissue transplant into a subject, wherein the subject is in need of the non-syngeneic cell or tissue transplant.

As used herein, the phrase “cell or tissue transplant” refers to a bodily cell (e.g. a single cell or a group of cells) or tissue (e.g. solid tissues/organs or soft tissues, which may be transplanted in full or in part). Exemplary tissues or organs which may be transplanted according to the present teachings include, but are not limited to, liver, pancreas, spleen, kidney, heart, lung, skin, intestine and lymphoid/hematopoietic tissues (e.g. lymph node, Peyer's patches thymus or bone marrow). Exemplary cells which may be transplanted according to the present teachings include, but are not limited to, immature hematopoietic cells, including stem cells, cardiac cells, hepatic cells, pancreatic cells, spleen cells, pulmonary cells, brain cells, nephric cells, intestine/gut cells, ovarian cells, skin cells, (e.g. isolated population of any of these cells). Furthermore, the present invention also contemplates transplantation of whole organs, such as for example, kidney, heart, liver or skin.

Depending on the application and available sources, the cells or tissues of the present invention may be obtained from a prenatal organism, postnatal organism, an adult organism or a cadaver donor. Moreover, depending on the application needed the cells or tissues may be naïve or genetically modified. Such determinations are well within the ability of one of ordinary skill in the art.

Any method known in the art may be employed to obtain a cell or tissue (e.g. for transplantation).

According to a specific embodiment, the cell or tissue transplant comprises immature hematopoietic cells.

As used herein the phrase “immature hematopoietic cells” refers to a hematopoietic tissue or cell preparation comprising precursor hematopoietic cells (e.g. hematopoietic stem cells). Such tissue/cell preparation includes or is derived from a biological sample, for example, bone marrow, mobilized peripheral blood (e.g. mobilization of CD34⁺ cells to enhance their concentration), cord blood (e.g. umbilical cord), fetal liver, yolk sac and/or placenta. Additionally, purified CD34⁺ cells or other hematopoietic stem cells such as CD131⁺ cells can be used in accordance with the present teachings, either with or without ex-vivo expansion.

According to one embodiment, the immature hematopoietic cells comprise T cell depleted immature hematopoietic cells.

As used herein the phrase “T cell depleted immature hematopoietic cells” refers to a population of precursor hematopoietic cells which are depleted of T lymphocytes. The T cell depleted immature hematopoietic cells, may include e.g. CD34⁺, CD33⁺ and/or CD56⁺ cells. The T cell depleted immature hematopoietic cells may be depleted of CD3⁺ cells, CD2⁺ cells, CD8⁺ cells, CD4⁺ cells, α/β T cells and/or γ/δ T cells.

According to one embodiment, the immature hematopoietic cells comprise T cell depleted mobilized blood cells enriched for CD34⁺ immature hematopoietic cells.

According to an embodiment, the T cell depleted immature hematopoietic cells comprise at least about 0.1×10⁶ CD34⁺ cells, 0.5×10⁶ CD34⁺ cells, 1×10⁶ CD34⁺ cells, 2×10⁶ CD34⁺ cells, 3×10⁶ CD34⁺ cells, 4×10⁶ CD34⁺ cells, 5×10⁶ CD34⁺ cells, 6×10⁶ CD34⁺ cells, 7×10⁶ CD34⁺ cells, 8×10⁶ CD34⁺ cells, 9×10⁶ CD34⁺ cells, 10×10⁶ CD34⁺ cells, 15×10⁶ CD34⁺ cells or 20×10⁶ CD34⁺ cells per kg ideal body weight of the subject.

According to a specific embodiment, the T cell depleted immature hematopoietic cells comprise at least about 2.5-20×10⁶ CD34⁺ cells (e.g. 5-10×10⁶ CD34⁺ cells) per kg ideal body weight of the subject.

According to a specific embodiment, the T cell depleted immature hematopoietic cells comprise at least about 5×10⁶ CD34⁺ cells per kg ideal body weight of the subject.

According to a specific embodiment, the T cell depleted immature hematopoietic cells comprise at least about 6×10⁶ CD34⁺ cells per kg ideal body weight of the subject.

According to a specific embodiment, the T cell depleted immature hematopoietic cells comprise at least about 8×10⁶ CD34⁺ cells per kg ideal body weight of the subject.

According to a specific embodiment, the T cell depleted immature hematopoietic cells comprise at least about 10×10⁶ CD34⁺ cells per kg ideal body weight of the subject.

According to one embodiment, the immature hematopoietic cells are depleted of CD3⁺ and/or CD19⁺ cells.

According to an embodiment, the T cell depleted immature hematopoietic cells comprise less than about 50×10⁵ CD3⁺ cells, 40×10⁵ CD3⁺ cells, 30×10⁵ CD3⁺ cells, 20×10⁵ CD3⁺ cells, 15×10⁵ CD3⁺ cells, 10×10⁵ CD3⁺ cells, 9×10⁵ CD3⁺ cells, 8×10⁵ CD3⁺ cells, 7×10⁵ CD3⁺ cells, 6×10⁵ CD3⁺ cells, 5×10⁵ CD3⁺ cells, 4×10⁵ CD3⁺ cells, 3×10⁵ CD3⁺ cells, 2×10⁵ CD3⁺ cells, 1×10⁵ CD3⁺ cells, 0.5×10⁵ CD3⁺ cells or 0.1×10⁵ CD3⁺ cells per kilogram ideal body weight of the subject.

According to a specific embodiment, the T cell depleted immature hematopoietic cells comprise less than 1-5×10⁵ CD3⁺ cells (e.g. 2-5×10⁵ CD3⁺ cells) per kilogram ideal body weight of the subject.

According to a specific embodiment, the T cell depleted immature hematopoietic cells comprise less than 5×10⁵ CD3⁺ cells per kilogram ideal body weight of the subject.

According to a specific embodiment, the T cell depleted immature hematopoietic cells comprise less than 4×10⁵ CD3⁺ cells per kilogram ideal body weight of the subject.

According to a specific embodiment, the T cell depleted immature hematopoietic cells comprise less than 3×10⁵ CD3⁺ cells per kilogram ideal body weight of the subject.

According to a specific embodiment, the T cell depleted immature hematopoietic cells comprise less than 2×10⁵ CD3⁺ cells per kilogram ideal body weight of the subject.

According to one embodiment, the immature hematopoietic cells are depleted of CD8⁺ cells.

According to an embodiment, the T cell depleted immature hematopoietic cells comprise less than 1×10⁴-5×10⁵ CD8⁺ cells (e.g. 0.1-4×10⁵ CD8⁺ cells, e.g. 1-3×10⁵ CD8⁺ cells) per kilogram ideal body weight of the subject.

According to an embodiment, the T cell depleted immature hematopoietic cells comprise less than about 50×10⁵ CD8⁺ cells, 25×10⁵ CD8⁺ cells, 15×10⁵ CD8⁺ cells, 10×10⁵ CD8⁺ cells, 9×10⁵ CD8⁺ cells, 8×10⁵ CD8⁺ cells, 7×10⁵ CD8⁺ cells, 6×10⁵ CD8⁺ cells, 5×10⁵ CD8⁺ cells, 4×10⁵ CD8⁺ cells, 3×10⁵ CD8⁺ cells, 2×10⁵ CD8⁺ cells, 1×10⁵ CD8⁺ cells, 9×10⁴ CD8⁺ cells, 8×10⁴ CD8⁺ cells, 7×10⁴ CD8⁺ cells, 6×10⁴ CD8⁺ cells, 5×10⁴ CD8⁺ cells, 4×10⁴ CD8⁺ cells, 3×10⁴ CD8⁺ cells, 2×10⁴ CD8⁺ cells or 1×10⁴ CD8⁺ cells per kilogram ideal body weight of the subject.

According to a specific embodiment, the T cell depleted immature hematopoietic cells comprise less than 5×10⁵ CD8⁺ cells per ideal kilogram body weight of the subject.

According to a specific embodiment, the T cell depleted immature hematopoietic cells comprise less than 4×10⁵ CD8⁺ cells per ideal kilogram body weight of the subject.

According to a specific embodiment, the T cell depleted immature hematopoietic cells comprise less than 3×10⁵ CD8⁺ cells per ideal kilogram body weight of the subject.

According to an embodiment, the T cell depleted immature hematopoietic cells comprise less than about 1×10⁶ CD8⁺ TCRα/β⁻ cells, 0.5×10⁶ CD8⁺ TCRα/β⁻ cells, 1×10⁵ CD8⁺ TCRα/β⁻ cells, 0.5×10⁵ CD8⁺ TCRα/β⁻ cells, 1×10⁴ CD8⁺ TCRα/β⁻ cells, 0.5×10⁴ CD8⁺ TCRα/β⁻ cells, 1×10³ CD8⁺ TCRα/β⁻ cells or 0.5×10³ CD8⁺ TCRα/β⁻ cells per kilogram ideal body weight of the subject.

According to a specific embodiment, the T cell depleted immature hematopoietic cells comprise less than 1×10⁵-1×10⁶ CD8⁺ TCRα/β⁻ cells per kilogram ideal body weight of the subject.

According to a specific embodiment, the T cell depleted immature hematopoietic cells comprise less than 1×10⁶ CD8⁺ TCRα/β⁻ cells per kilogram ideal body weight of the subject.

According to a specific embodiment, the T cell depleted immature hematopoietic cells comprise less than 5×10⁵ CD8⁺ TCRα/β⁻ cells per kilogram ideal body weight of the subject.

According to a specific embodiment, the T cell depleted immature hematopoietic cells comprise less than 1×10⁵ CD8⁺ TCRα/β⁻ cells per kilogram ideal body weight of the subject.

According to one embodiment, the immature hematopoietic cells are depleted of B cells.

According to an embodiment, the immature hematopoietic cells are depleted of B cells (CD19⁺ and/or CD20⁺ B cells).

According to an embodiment, the immature hematopoietic cells comprise less than about 50×10⁵ CD19⁺ and/or CD20⁺ cells, 40×10⁵ CD19⁺ and/or CD20⁺ cells, 30×10⁵ CD19⁺ and/or CD20⁺ cells, 20×10⁵ CD19⁺ and/or CD20⁺ cells, 10×10⁵ CD19⁺ and/or CD20⁺ cells, 9×10⁵ CD19⁺ and/or CD20⁺ cells, 8×10⁵ CD19⁺ and/or CD20⁺ cells, 7×10⁵ CD19⁺ and/or CD20⁺ cells, 6×10⁵ CD19⁺ and/or CD20⁺ cells, 5×10⁵ CD19⁺ and/or CD20⁺ cells, 4×10⁵ CD19⁺ and/or CD20⁺ cells, 3×10⁵ CD19⁺ and/or CD20⁺ cells, 2×10⁵ CD19⁺ and/or CD20⁺ cells or 1×10⁵ CD19⁺ and/or CD20⁺ cells per kilogram ideal body weight of the subject.

According to a specific embodiment, the immature hematopoietic cells comprise less than 1-5×10⁵ CD19⁺ and/or CD20⁺ cells (e.g. 3-5×10⁵ CD19⁺ and/or CD20⁺ cells) per kilogram ideal body weight of the subject.

According to a specific embodiment, the immature hematopoietic cells comprise less than 4×10⁵ CD19⁺ and/or CD20⁺ cells per kilogram ideal body weight of the subject.

According to a specific embodiment, the immature hematopoietic cells comprise less than 3×10⁵ CD19⁺ and/or CD20⁺ cells per kilogram ideal body weight of the subject.

According to a specific embodiment, the immature hematopoietic cells comprise less than 2×10⁵ CD19⁺ and/or CD20⁺ cells per kilogram ideal body weight of the subject.

According to a specific embodiment, the T cell depleted immature hematopoietic cells comprise two or more batches of cells, e.g. a first batch comprising CD34+ selected cells and a second batch comprising CD3⁺/CD19⁺-depleted cells (i.e. obtained from the same donor). It will be appreciated that these can be used concomitantly or subsequent to each other (e.g. on the same day or within e.g. about 1, 2, 3, 4, 5, 6, 7 days of each other, as discussed below).

Depletion of T cells, e.g. CD3⁺, CD2⁺, TCRα/β⁺, CD4⁺ and/or CD8⁺ cells, or B cells, e.g. CD19⁺ and/or CD20⁺ cells, may be carried out using any method known in the art, such as by eradication (e.g. killing) with specific antibodies or by affinity based purification e.g. such as by the use of magnetic cell separation techniques, FACS sorter and/or capture ELISA labeling.

Such methods are described herein and in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 1992). For example, cells can be sorted by, for example, flow cytometry or FACS. Thus, fluorescence activated cell sorting (FACS) may be used and may have varying degrees of color channels, low angle and obtuse light scattering detecting channels, and impedance channels. Any ligand-dependent separation techniques known in the art may be used in conjunction with both positive and negative separation techniques that rely on the physical properties of the cells rather than antibody affinity, including but not limited to elutriation and density gradient centrifugation.

Other methods for cell sorting include, for example, panning and separation using affinity techniques, including those techniques using solid supports such as plates, beads and columns. Thus, biological samples may be separated by “panning” with an antibody attached to a solid matrix, e.g. to a plate.

Alternatively, cells may be sorted/separated by magnetic separation techniques, and some of these methods utilize magnetic beads. Different magnetic beads are available from a number of sources, including for example, Dynal (Norway), Advanced Magnetics (Cambridge, MA, U.S.A.), Immuncon (Philadelphia, U.S.A.), Immunotec (Marseille, France), Invitrogen, Stem cell Technologies (U.S.A) and Cellpro (U.S.A). Alternatively, antibodies can be biotinylated or conjugated with digoxigenin and used in conjunction with avidin or anti-digoxigenin coated affinity columns.

According to an embodiment, different depletion/separation methods can be combined, for example, magnetic cell sorting can be combined with FACS, to increase the separation quality or to allow sorting by multiple parameters.

According to a specific embodiment, T cell depleted immature hematopoietic cells are obtained by a method comprising collecting mobilized PBMCs from a donor subject (e.g. the same donor subject from which non-mobilized PBMCs were collected for generation of non-GVHD inducing cells comprising a Tcm phenotype).

According to one embodiment, mobilization is affected by G-CSF.

According to one embodiment, mobilization is affected by G-CSF and plerixafor.

According to a specific embodiment, the collection of mobilized PBMCs is obtained in a single collection.

According to a specific embodiment, the collection of the mobilized PBMCs is obtained in two, three, four, five or more daily collection, e.g. three daily collections (e.g. on consequent days or within a few days apart).

According to a specific embodiment, a back-up fraction of unmodified mobilized PBMC containing at least about 0.1-5×10⁶ CD34⁺ cells, e.g. 2×10⁶ CD34⁺ cells, e.g. 1×10⁶ CD34⁺ cells per kg ideal body weight (e.g. obtained from the first collection) is set aside and cryopreserved.

According to a specific embodiment, the collections of mobilized PBMCs (e.g. the remaining from the first and the second day's collections) are pooled, CD34⁺ selected (as discussed below) and cryopreserved.

According to a specific embodiment, the third day's collection is depleted of CD3⁺/CD19⁺ cells (as discussed below) and cryopreserved.

According to one embodiment, enrichment of CD34⁺ expressing cells is affected by incubating the PBMCs with a CD34 binding agent.

According to a specific embodiment, the CD34 binding agent is an antibody.

According to a specific embodiment, the CD34 antibody is a monoclonal antibody.

According to a specific embodiment, the CD34 monoclonal antibody is conjugated to magnetic particles.

According to a specific embodiment, the CD34 monoclonal antibody is conjugated to super-paramagnetic particles.

According to one embodiment, the CD34⁺ labeled cells are selected by magnetic separation techniques (as discussed in detail hereinabove).

According to a specific embodiment, the CD34 magnetically labeled cells (i.e. CD34⁺ expressing cells) are retained by the separation column (i.e. positive selection) and the CD34⁻ cells are removed. The CD34⁺ cells are then released from the column and collected. According to one embodiment, samples from each fraction are removed for cell count, viability and/or immunophenotyping.

According to one embodiment, the mobilized PBMCs are depleted of platelets using e.g. COBE 2991.

According to one embodiment, the post-platelet depleted PBMC preparation of one embodiment is incubated with IVIg for 5-30 minutes e.g. 10-15 minutes. After the initial incubation the CD3⁺ and CD19⁺ binding agents are added to the cell preparation and incubated for e.g. 10-60 minutes, e.g. 30 minutes, on an orbital rotator.

According to a specific embodiment, the CD3 and/or CD19 binding agent is an antibody.

According to a specific embodiment, the CD3 and/or CD19 antibody is a monoclonal antibody.

According to a specific embodiment, the CD3 and/or CD19 monoclonal antibody is conjugated to magnetic particles.

According to a specific embodiment, the CD3 and/or CD19 monoclonal antibody is conjugated to super-paramagnetic particles.

According to one embodiment, at the end of the incubation, the cells are washed by centrifugation and the cell pellet resuspended in buffer (e.g. COBE 2991) to remove excess reagent.

According to one embodiment, the CD3⁺/CD19⁺ labeled cells are selected by magnetic separation techniques (as discussed in detail hereinabove).

According to a specific embodiment, the CD3⁺/CD19⁺ labeled cells are processed on CliniMACS® column.

According to a specific embodiment, the CD3⁺/CD19⁺ magnetically labeled cells (i.e. CD3⁺/CD19⁺ expressing cells) are retained by the separation column (i.e. negative selection) and the CD4⁻/CD56⁻/CD45RA⁻ cells are collected. According to one embodiment, the collected cells (CD3⁻/CD19⁻ cells) are washed and collected. According to one embodiment, samples from each fraction are removed for cell count, viability and/or immunophenotyping.

According to a specific embodiment, a second CD3⁺ depletion step is carried out (as discussed above) in situations in which more than about 1×10⁵ CD3 to 5×10⁵ CD3, e.g. 1×10⁵ CD3 to 3×10⁵ CD3, e.g. 2.5×10⁵ CD3, e.g. 2×10⁵ CD3, per kg ideal body weight are present in the collected cell fraction.

Alternatively infusion of only part of the T cell depleted fraction is utilized so as to avoid infusion of more than 5×10⁵ CD3, e.g. 3×10⁵ CD3, e.g. 2×10⁵ CD3 cells/kg ideal body weight.

According to one embodiment, the T cell depleted immature hematopoietic cells (i.e. for cell transplantation) and the PBMCs used for generation of the non-GVHD inducing cells comprising a Tcm phenotype (i.e. for generation of veto cells) are obtained from the same donor subject.

Depending on the application, the method may be affected using a cell or tissue which is syngeneic or non-syngeneic with the recipient subject (e.g. allogeneic), as discussed hereinabove.

According to an embodiment of the present invention, both the recipient subject and the donor subject are humans.

According to an embodiment of the present invention, the subject (i.e. recipient subject) suffers a disease which can be treated by transplantation of a cell or tissue graft.

According to an embodiment of the present invention, the subject (i.e. recipient subject) suffers a disease which can be treated by transplantation of an organ or tissues.

According to an embodiment of the present invention, the subject (i.e. recipient subject) suffers from a disease which can be treated by immature hematopoietic cell transplantation (e.g. T cell depleted immature hematopoietic cells).

According to an embodiment of the present invention, the subject (i.e. recipient subject) suffers a disease which can be treated by co-transplantation of immature hematopoietic cells and a solid organ or tissue (e.g. kidney, pulmonary, liver, pancreatic, cardiac, etc.).

According to an embodiment of the present invention, the subject (i.e. recipient subject) suffers a disease which can be treated by co-transplantation of immature hematopoietic cells and isolated cells obtained from a solid organ or tissue (e.g. kidney, pulmonary cells, liver cells, pancreatic cells, cardiac cells, etc. as discussed above).

According to an embodiment of the present invention, the subject (i.e. recipient subject) suffers a disease which can be treated by co-transplantation of several organs (e.g. cardiac and pulmonary tissues).

According to one embodiment, the cells, organs or tissues for co-transplantation are obtained from the same donor.

Transplanting the cell or tissue into the subject may be affected in numerous ways, depending on various parameters, such as, for example, the cell or tissue type; the type, stage or severity of the recipient's disease (e.g. type and stage of malignant disease); the physical or physiological parameters specific to the subject; and/or the desired therapeutic outcome.

Transplanting a cell or tissue transplant of the present invention may be affected by transplanting the cell or tissue transplant into any one of various anatomical locations, depending on the application. The cell or tissue transplant may be transplanted into a homotopic anatomical location (a normal anatomical location for the transplant), or into an ectopic anatomical location (an abnormal anatomical location for the transplant). Depending on the application, the cell or tissue transplant may be advantageously implanted under the renal capsule, or into the kidney, the testicular fat, the sub cutis, the omentum, the portal vein, the liver, the spleen, the heart cavity, the heart, the chest cavity, the lung, the skin, the pancreas and/or the intra-abdominal space.

The immature hematopoietic cells (e.g. T cell depleted immature hematopoietic cells) of some embodiments of the invention may be transplanted into a subject using any method known in the art for cell transplantation, such as but not limited to, cell infusion (e.g. I.V.) or via an intraperitoneal route.

Following transplantation of the cell or tissue transplant into the subject according to the present teachings, it is advisable, according to standard medical practice, to monitor the growth functionality and immuno-compatibility of the cells, tissues or organs according to any one of various standard art techniques. For example, the cell numbers of immature hematopoietic cells can be monitored in a subject by standard blood and bone marrow tests (e.g. by FACS analysis). Structural development of the cells or tissues may be monitored via computerized tomography, or ultrasound imaging.

Depending on the transplantation context, in order to facilitate engraftment of the cell or tissue transplant, the method may further advantageously comprise conditioning the subject under sublethal, lethal or supralethal conditions prior to the transplanting.

As used herein, the terms “sublethal”, “lethal”, and “supralethal”, when relating to conditioning of subjects of the present invention, refer to myelotoxic and/or lymphocytotoxic treatments which, when applied to a representative population of the subjects, respectively, are typically: non-lethal to essentially all members of the population; lethal to some but not all members of the population; or lethal to essentially all members of the population under normal conditions of sterility.

According to some embodiments of the invention, the sublethal, lethal or supralethal conditioning comprises a total body irradiation (TBI), total lymphoid irradiation (TLI, i.e. exposure of all lymph nodes, the thymus, and spleen), partial body irradiation (e.g. specific exposure of the lungs, kidney, brain etc.), myeloablative conditioning and/or non-myeloablative conditioning, e.g. with different combinations including, but not limited to, co-stimulatory blockade, chemotherapeutic agent and/or antibody immunotherapy. According to some embodiments of the invention, the conditioning comprises a combination of any of the above described conditioning protocols (e.g. chemotherapeutic agent and TBI, co-stimulatory blockade and chemotherapeutic agent, antibody immunotherapy and chemotherapeutic agent, etc).

According to one embodiment, the conditioning is affected by conditioning the subject under supralethal conditions, such as under myeloablative conditions (i.e. intensive conditioning regimen in which the bone marrow cells are destroyed).

Alternatively, the conditioning may be affected by conditioning the subject under lethal or sublethal conditions, such as by conditioning the subject under myeloreductive conditions or non-myeloablative conditions, respectively (i.e. reduced intensity conditioning which is a less aggressive conditioning regimen).

According to a specific embodiment, the conditioning comprises non-myeloablative conditioning (e.g. a reduced intensity conditioning regimen).

According to an embodiment, the reduced intensity conditioning is affected for up to 2 weeks (e.g. 1-10 or 1-7 days) prior to transplantation of the cell or tissue graft.

According to a specific embodiment, the non-myeloablative conditioning comprises a chemotherapeutic agent and TBI/TLI.

According to one embodiment, the TBI comprises a single or fractionated irradiation dose within the range of 0.5-1 Gy, 0.5-1.5 Gy, 0.5-2 Gy, 0.5-2.5 Gy, 0.5-5 Gy, 0.5-7.5 Gy, 0.5-10 Gy, 0.5-15 Gy, 1-1.5 Gy, 1-2 Gy, 1-2.5 Gy, 1-3 Gy, 1-3.5 Gy, 1-4 Gy, 1-4.5 Gy, 1-5 Gy, 1-7.5 Gy, 1-10 Gy, 2-3 Gy, 2-4 Gy, 2-5 Gy, 2-6 Gy, 2-7 Gy, 2-8 Gy, 2-9 Gy, 2-10 Gy, 3-4 Gy, 3-5 Gy, 3-6 Gy, 3- 7 Gy, 3-8 Gy, 3-9 Gy, 3-10 Gy, 4-5 Gy, 4-6 Gy, 4-7 Gy, 4-8 Gy, 4-9 Gy, 4-10 Gy, 5-6 Gy, 5-7 Gy, 5-8 Gy, 5-9 Gy, 5-10 Gy, 6-7 Gy, 6-8 Gy, 6-9 Gy, 6-10 Gy, 7-8 Gy, 7-9 Gy, 7-10 Gy, 8-9 Gy, 8-10 Gy, 10-12 Gy or 10-15 Gy.

According to a specific embodiment, the TBI comprises a single or fractionated irradiation dose within the range of 1-7.5 Gy.

According to a specific embodiment, the TBI comprises a single or fractionated irradiation dose within the range of 1-5 Gy.

According to a specific embodiment, the TBI comprises a single or fractionated irradiation dose of 3 Gy.

According to an embodiment, TBI treatment is administered to the subject 1-10 days (e.g. 1-3 days) prior to transplantation. According to one embodiment, the subject is conditioned once with TBI 1, 2, 3 or 4 days prior to transplantation.

According to an embodiment, TBI is administered on day −2 prior to transplantation.

According to an embodiment, TBI is administered on day −1 prior to transplantation.

According to an embodiment, TBI is administered on the day of transplantation, e.g. in the morning of day 0, and transplantation is carried out on the same day, e.g. in the evening.

According to a specific embodiment, the TBI comprises a single or fractionated irradiation dose on day −1 (i.e. one day prior to transplantation).

According to a specific embodiment, the TLI comprises an irradiation dose within the range of 0.5-1 Gy, 0.5-1.5 Gy, 0.5-2.5 Gy, 0.5-5 Gy, 0.5-7.5 Gy, 0.5-10 Gy, 0.5-15 Gy, 1-1.5 Gy, 1-2 Gy, 1-2.5 Gy, 1-3 Gy, 1-3.5 Gy, 1-4 Gy, 1-4.5 Gy, 1-1.5 Gy, 1-7.5 Gy, 1-10 Gy, 2-3 Gy, 2-4 Gy, 2-5 Gy, 2-6 Gy, 2-7 Gy, 2-8 Gy, 2-9 Gy, 2-10 Gy, 3-4 Gy, 3-5 Gy, 3-6 Gy, 3-7 Gy, 3-8 Gy, 3-9 Gy, 3- 10 Gy, 4-5 Gy, 4-6 Gy, 4-7 Gy, 4-8 Gy, 4-9 Gy, 4-10 Gy, 5-6 Gy, 5-7 Gy, 5-8 Gy, 5-9 Gy, 5-10 Gy, 6-7 Gy, 6-8 Gy, 6-9 Gy, 6-10 Gy, 7-8 Gy, 7-9 Gy, 7-10 Gy, 8-9 Gy, 8-10 Gy, 10-12 Gy, 10-15 Gy, 10-20 Gy, 10-30 Gy, 10-40 Gy, 10-50 Gy, 0.5-20 Gy, 0.5-30 Gy, 0.5-40 Gy or 0.5-50 Gy.

According to a specific embodiment, the TLI comprises a single or fractionated irradiation dose within the range of 1-7.5 Gy.

According to a specific embodiment, the TLI comprises a single or fractionated irradiation dose within the range of 1-5 Gy.

According to a specific embodiment, the TLI comprises a single or fractionated irradiation dose of 3 Gy.

According to an embodiment, TLI treatment is administered to the subject 1-10 days (e.g. 1-3 days) prior to transplantation. According to one embodiment, the subject is conditioned once with TLI 1, 2, 3 or 4 days prior to transplantation.

According to an embodiment, TLI is administered on day −2 prior to transplantation.

According to an embodiment, TLI is administered on day −1 prior to transplantation.

According to an embodiment, TLI is administered on the day of transplantation, e.g. in the morning of day 0, and transplantation is carried out on the same day, e.g. in the evening.

According to a specific embodiment, the TLI comprises a single or fractionated irradiation dose on day −1 (i.e. one day prior to transplantation).

According to one embodiment, the conditioning comprises a chemotherapeutic agent. Exemplary chemotherapeutic agents include, but are not limited to, Busulfan, Busulfex, Cyclophosphamide, Fludarabine, Melphalan, Myleran, Rapamycin, Trisulphan, and Thiotepa. The chemotherapeutic agent/s may be administered to the subject in a single dose or in several doses e.g. 2, 3, 4, 5, 6, 7, 8, 9, 10 or more doses (e.g. daily doses) prior to transplantation.

According to a specific embodiment, the subject is administered a chemotherapeutic agent (e.g. Fludarabine e.g. at a dose of about 30 mg/m²/day) for 3, 4, 5 or 6 consecutive days (e.g. 4 consecutive days) prior to transplantation (e.g. on days −7 to −4, e.g. on days −6 to −3).

Fludarabine is commercially available from e.g. Sanofi Genzyme, Bayer and Teva, e.g. under the brand name e.g. Fludara.

According to one embodiment, the pre-transplant conditioning comprises in vivo T cell debulking.

According to some embodiments, the in-vivo T cell debulking is affected by antibodies.

According to some embodiments of the invention, the antibodies comprise an anti-CD8 antibody, an anti-CD4 antibody, or both.

According to some embodiments of the invention, the antibodies comprise anti-thymocyte globulin (ATG) antibodies, anti-CD52 antibodies or anti-CD3 (OKT3) antibodies.

According to one embodiment, the antibody is administered to the subject in a single dose or in several doses e.g. 2, 3, 4, 5, 6, 7, 8, 9, 10 or more doses (e.g. daily doses) prior to transplantation.

According to a specific embodiment, the subject is administered an antibody therapeutic agent (e.g. ATG e.g. at a dose of about 2 mg per kg ideal body weight) for 2, 3, 4 or 5 consecutive days (e.g. 3 consecutive days) prior to transplantation (e.g. on days −10 to −8 prior to transplantation, e.g. on days −9 to −7 prior to transplantation).

According to a specific embodiment, the subject is treated with three administrations of ATG prior to transplantation (e.g. at a dose of about 2 mg per kg ideal body weight, e.g. on days −9, −8 and −7 prior to transplantation).

According to a specific embodiment, the subject is treated with two administrations of ATG prior to transplantation (e.g. at a dose of about 2 mg per kg ideal body weight, e.g. on days −9 and −8, or −8 and −7 prior to transplantation).

According to a specific embodiment, the subject is treated with a single administration of ATG prior to transplantation (e.g. at a dose of about 2 mg per kg ideal body weight, e.g. on days −9, −8 or −7 prior to transplantation).

According to a specific embodiment, the pre-transplant conditioning does not comprise in vivo T cell debulking.

According to a specific embodiment, the subject is not treated with ATG prior to transplantation.

It will be appreciated that when using no ATG or lower doses of ATG are used, e.g. single dose or two doses (e.g. each at a dose of about 2 mg per kg ideal body weight), higher radiation doses can be used as part of the non-myeloablative conditioning protocol (e.g. TBI at a single or fractionated irradiation dose of 3-5 Gy, e.g. 3 Gy, 4 Gy or 5 Gy).

Anti-thymocyte globulin (ATG) antibodies are commercially available from e.g. Genzyme and Pfizer, e.g. under the brand names e.g. Thymoglobulin and Atgam.

According to one embodiment, when the subject has a B-cell malignancy, the pre-transplant conditioning regimen comprises rituximab (e.g. at a dose of about 375 mg/m², e.g. on days −12, −11, −10, −9, −8 or −7, e.g. on day −10 prior to transplantation).

Rituximab is commercially available from e.g. Genentech and Roche, e.g. under the brand names e.g. Mabthera, Rixathon, Truxima, Rituxan.

According to one embodiment, the method comprises post-transplant administration of a therapeutically effective amount of cyclophosphamide.

According to one embodiment, the present invention further contemplates administration of cyclophosphamide prior to transplantation (e.g. on days 6, 5, 4 or 3 prior to transplantation, i.e. D−6 to −3) in addition to the administration following transplantation as described herein.

For example, in case of cell or tissue graft, the therapeutic effective amount of cyclophosphamide comprises about 1-25 mg, 1-50 mg, 1-75 mg, 1-100 mg, 1-250 mg, 1-500 mg, 1-750 mg, 1-1000 mg, 5-50 mg, 5-75 mg, 5-100 mg, 5-250 mg, 5-500 mg, 5-750 mg, 5-1000 mg, 10-50 mg, 10-75 mg, 10-100 mg, 10-250 mg, 10-500 mg, 10-750 mg, 10-1000 mg, 25-50 mg, 25-75 mg, 25-100 mg, 25-125 mg, 25-200 mg, 25-300 mg, 25-400 mg, 25-500 mg, 25-750 mg, 25-1000 mg, 50-75 mg, 50-100 mg, 50-125 mg, 50-150 mg, 50-175 mg, 50-200 mg, 50-250 mg, 50-500 mg, 50-1000 mg, 75-100 mg, 75-125 mg, 75-150 mg, 75-250 mg, 75-500 mg, 75-1000 mg, 100-125 mg, 100-150 mg, 100-200 mg, 100-300 mg, 100-400 mg, 100-500 mg, 100-1000 mg, 125-150 mg, 125-250 mg, 125-500 mg, 125-1000 mg, 150-200 mg, 150-300 mg, 150-500 mg, 150-1000 mg, 200-300 mg, 200-400 mg, 200-500 mg, 200-750 mg, 200-1000 mg, 250-500 mg, 250-750 mg, 250-1000 mg per kilogram ideal body weight of the subject.

According to a specific embodiment, the therapeutic effective amount of cyclophosphamide is about 25-200 mg per kilogram ideal body weight of the subject.

According to one embodiment, cyclophosphamide is administered in a single dose.

According to one embodiment, cyclophosphamide is administered in multiple doses, e.g. in 2, 3, 4, 5 doses or more.

According to a specific embodiment, cyclophosphamide is administered in two doses.

The dose of each cyclophosphamide administration may comprise about 5 mg, 7.5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg or 500 mg per kilogram ideal body weight of the subject.

According to a specific embodiment, each dose of cyclophosphamide is 50 mg per kilogram ideal body weight of the subject.

As mentioned, cyclophosphamide is administered post transplantation. Thus, for example, cyclophosphamide may be administered to the subject 1, 2, 3, 4, 5 days post-transplant (i.e., D+1, +2, +3, +4, +5).

According to a specific embodiment, cyclophosphamide is administered to the subject in two doses 3 and 4 days post-transplant.

Cyclophosphamide is commercially available from e.g. Zydus (German Remedies), Roxane Laboratories Inc-Boehringer Ingelheim, Bristol-Myers Squibb Co—Mead Johnson and Co, and Pfizer—Pharmacia & Upjohn, under the brand names of Endoxan, Cytoxan, Neosar, Procytox and Revimmune.

According to one embodiment, the subject is treated with additional supportive drugs, e.g. chemotherapy adjuvants.

According to one embodiment, the subject is treated with a dose of Mesna (e.g. 10 mg/kg intravenous piggy back (IVPB) just prior to the first dose of cyclophosphamide (e.g. 2 hours, 1 hour, 30 minutes, 15 minutes prior to the first dose of cyclophosphamide). According to one embodiment, administration of mesna is repeated every 4 hours for a total of 10 doses.

Mesna is commercially available from e.g. Baxter under the brand names of Uromitexan and Mesnex.

According to a one embodiment, the subject is treated with ondansetron (or another anti-emetic) prior to each dose of Cyclophosphamide (Cy).

According to one embodiment, the subject is not treated with an immunosuppressive agent (e.g. aside from the CY and veto cells discussed herein).

According to one embodiment, the subject is treated with an immunosuppressive agent.

Examples of immunosuppressive agents include, but are not limited to, Tacrolimus (also referred to as FK-506 or fujimycin, trade names: Prograf, Advagraf, Protopic), Mycophenolate Mofetil, Mycophenolate Sodium, Prednisone, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, tramadol, rapamycin (sirolimus) and rapamycin analogs (such as CCI-779, RAD001, AP23573). These agents may be administered individually or in combination.

According to one embodiment, corticosteroids are not administered as a pretreatment to the veto cells.

According to another aspect of the invention, there is provided a method of treating a subject in need of an immature hematopoietic cell transplantation, the method comprising:
(a) conditioning the subject under non-myeloablative pre-transplant conditioning protocol, wherein the non-myeloablative conditioning comprises a total body irradiation (TBI) and a chemotherapeutic agent, wherein the TBI and the chemotherapeutic agent are administered on days −7 to −1 prior to transplantation (e.g. on days −7 to −2, or on days −6 to −1 prior to transplantation);
(b) transplanting into the subject a dose of T cell depleted immature hematopoietic cells, wherein the T cell depleted immature hematopoietic cells comprises less than 5×10⁵ CD3⁺ T cells per kilogram ideal body weight of the subject, and wherein the T cell depleted immature hematopoietic cells comprise at least 5×10⁶ CD34⁺ cells per kilogram ideal body weight of the subject;
(c) administering to the subject a therapeutically effective amount of cyclophosphamide, wherein the therapeutically effective amount of the cyclophosphamide comprises 25-200 mg cyclophosphamide per kilogram ideal body weight of the subject, and wherein the therapeutically effective amount of the cyclophosphamide is to be administered to the subject in two doses 3 and 4 days following the transplantation of the T cell depleted immature hematopoietic cells; and
(d) administering to the subject a therapeutically effective amount of the isolated population of non-GVHD inducing cells of some embodiments of the invention, wherein the isolated population of non-GVHD inducing cells are administered on day 7 following the transplantation of the T cell depleted immature hematopoietic cells, thereby treating the subject in need of the immature hematopoietic cell transplantation.

According to a specific embodiment, TBI is administered on the day of transplantation of the T cell depleted immature hematopoietic cells e.g. in the morning of day 0, and transplantation is carried out on the same day, e.g. in the evening.

According to a specific embodiment, the subject is treated as follows: Fludarabine is administered to the subject in 4 consequetive doses (e.g. 30 mg/m²/day) on days −6 to −3 (prior to transplantation), TBI is administered to the subject at a single or fractionated irradiation dose (e.g. of 3-5 Gy, e.g. 3 Gy) on day −1 prior to transplantation, on day 0 (i.e. transplantation day) the subject is infused (e.g. IV) with megadose T cell depleted immature hematopoietic cells comprising CD34⁺ selected cells and/or CD3/CD19-depleted cells (e.g. cells collected after mobilization of a donor, depleted of CD3/CD19 expressing cells and/or selected for expression of CD34, cryopreserved and thawed on the day of the transplant, as described herein), on days +3 and +4 the subject is administered (e.g. IV) with Cyclophosphamide (CY) each at a dose of e.g. 50 mg/kg ideal body weight/day, and on day +7 the subject is infused with the anti-viral veto cells of some embodiments of the invention at a dose of e.g. 2.5-10×10⁶ CD8⁺ cells per kg ideal body weight.

According to a specific embodiment, the subject is administered with ATG (Thymoglobulin) in 3 consequetive doses (e.g. 2 mg per kg ideal body weight) on days −9 to −7 (prior to transplantation).

According to a specific embodiment, the subject is treated as follows: Fludarabine is administered to the subject in 4 consequetive doses (e.g. 30 mg/m²/day) on days −7 to −4 (prior to transplantation), TBI is administered to the subject at a single or fractionated irradiation dose (e.g. of 3-5 Gy, e.g. 3 Gy) on day −2 prior to transplantation, on day −1 the subject is infused (e.g. IV) with a first dose of megadose T cell depleted immature hematopoietic cells comprising CD34⁺ selected cells (e.g. cells collected after mobilization of a donor and selected for expression of CD34 and used as fresh cells, as described herein), on day 0 the subject is infused (e.g. IV) with a second dose of megadose T cell depleted immature hematopoietic cells comprising CD3/CD19-depleted cells (e.g. cells collected after mobilization of a donor, depleted of CD3/CD19 expressing cells and used as fresh cells as described herein), on days +3 and +4 the subject is administered (e.g. IV) with Cyclophosphamide (CY) each at a dose of e.g. 50 mg/kg ideal body weight/day, and on day +7 the subject is infused with the anti-viral veto cells of some embodiments of the invention at a dose of e.g. 2.5-10×10⁶ CD8⁺ cells per kg ideal body weight.

According to a specific embodiment, the subject is administered with ATG (Thymoglobulin) in 3 consequetive doses (e.g. 2 mg per kg ideal body weight) on days −10 to −8 (prior to transplantation).

According to a specific embodiment, the subject is treated with a dose of Mesna (e.g. 10 mg/kg intravenous piggy back (IVPB) just prior to the first dose of cyclophosphamide. According to one embodiment, this is repeated every 4 hours for a total of 10 doses.

According to a specific embodiment, the subject is treated with ondansetron (or another anti-emetic) prior to each dose of Cyclophosphamide (CY).

According to a specific embodiment, premedication for the veto cells does not include corticosteroids.

According to a specific embodiment, when the subject has a B-cell malignancy, the pre-transplant conditioning regimen comprises rituximab (e.g. at a dose of about 375 mg/m2, e.g. on day −10 prior to transplantation).

The number of administrations and the therapeutically effective amount of the pre-transplant and post-transplant immunosuppressive drugs and/or immunosuppressive irradiation may be adjusted as needed taking into account the type of transplantation and the subject's response to the regimen. Determination of the number of administrations and the therapeutically effective amount is well within the capability of those skilled in the art, especially in light of the detailed disclosure provided herein.

Regardless of the transplant type, to avoid graft rejection and graft versus host disease (GHVD) and/or to induce donor specific tolerance, the method of the present invention utilizes the novel Tcm cells (as described in detail hereinabove).

According to one embodiment, the non-GVHD inducing cells comprising a Tcm phenotype (i.e. veto cells) of some embodiments of the invention are used per se for reduction of graft rejection and or for reduction of GVHD of transplanted cells, tissues or organs (e.g. transplanted from the same donor).

According to one embodiment, the non-GVHD inducing cells comprising a Tcm phenotype (i.e. veto cells) are for administration either concomitantly with, prior to, or following the transplantation of the cell or tissue transplant.

According to one embodiment, the non-GVHD inducing cells comprising a Tcm phenotype (i.e. veto cells) are for administration either concomitantly with, prior to, or following the transplantation of immature hematopoietic cells (e.g. T cell depleted immature hematopoietic cells).

According to a specific embodiment, the non-GVHD inducing cells comprising a Tcm phenotype (i.e. veto cells) are for administration following the non-syngeneic cell or tissue transplant.

According to a specific embodiment, the non-GVHD inducing cells comprising a Tcm phenotype (i.e. veto cells) are for administration following the immature hematopoietic cells (e.g. T cell depleted immature hematopoietic cells).

According to a one embodiment, the non-GVHD inducing cells comprising a Tcm phenotype (i.e. veto cells) are for administration on day 3, 4, 5, 6, 7, 8, 9, 10, 12, 14, 21 following the non-syngeneic cell or tissue transplant (e.g. T cell depleted immature hematopoietic cells).

According to a one embodiment, the non-GVHD inducing cells comprising a Tcm phenotype (i.e. veto cells) are for administration on day 3, 4, 5, 6, 7, 8, 9, 10, 12, 14, 21 following the immature hematopoietic cells (e.g. T cell depleted immature hematopoietic cells).

According to a specific embodiment, the non-GVHD inducing cells comprising a Tcm phenotype (i.e. veto cells) are for administration on day 6-9 following the non-syngeneic cell or tissue transplant (e.g. immature hematopoietic cells e.g. T cell depleted immature hematopoietic cells).

According to a specific embodiment, the non-GVHD inducing cells comprising a Tcm phenotype (i.e. veto cells) are for administration on day 9 following the non-syngeneic cell or tissue transplant.

According to a specific embodiment, the non-GVHD inducing cells comprising a Tcm phenotype (i.e. veto cells) are for administration on day 8 following the non-syngeneic cell or tissue transplant.

According to a specific embodiment, the non-GVHD inducing cells comprising a Tcm phenotype (i.e. veto cells) are for administration on day 7 following the non-syngeneic cell or tissue transplant.

According to a specific embodiment, the non-GVHD inducing cells comprising a Tcm phenotype (i.e. veto cells) are for administration on day 6 following the non-syngeneic cell or tissue transplant.

According to a specific embodiment, the non-GVHD inducing cells comprising a Tcm phenotype (i.e. veto cells) are for administration on day 9 following the immature hematopoietic cells (e.g. T cell depleted immature hematopoietic cells).

According to a specific embodiment, the non-GVHD inducing cells comprising a Tcm phenotype (i.e. veto cells) are for administration on day 8 following the immature hematopoietic cells (e.g. T cell depleted immature hematopoietic cells).

According to a specific embodiment, the non-GVHD inducing cells comprising a Tcm phenotype (i.e. veto cells) are for administration on day 7 following the immature hematopoietic cells (e.g. T cell depleted immature hematopoietic cells).

According to a specific embodiment, the non-GVHD inducing cells comprising a Tcm phenotype (i.e. veto cells) are for administration on day 6 following the immature hematopoietic cells (e.g. T cell depleted immature hematopoietic cells).

According to a specific embodiment, when veto cells are used as adjuvant treatment for transplantation of cells or tissues obtained from a solid organ or tissue (e.g. kidney, pulmonary cells, liver cells, pancreatic cells, cardiac cells, etc. as discussed above), the veto cells may be administered concomitantly with, prior to, or following the transplantation of the cell or tissue transplant.

According to a specific embodiment, when co-transplantation of immature hematopoietic cells (e.g. T cell depleted immature hematopoietic cells) and cells or tissues obtained from a solid organ or tissue (e.g. kidney, pulmonary cells, liver cells, pancreatic cells, cardiac cells, etc. as discussed above) is carried out, the veto cells, the immature hematopoietic cells and the cells or tissues can be administered in any order (e.g. kidney, immature hematopoietic cells and veto cells; immature hematopoietic cells, kidney and veto cells; etc).

According to a specific embodiment, when co-transplantation of immature hematopoietic cells (e.g. T cell depleted immature hematopoietic cells) and cells or tissues obtained from a solid organ or tissue (e.g. kidney, pulmonary cells, liver cells, pancreatic cells, cardiac cells, etc. as discussed above) is carried out, the veto cells and the immature hematopoietic cells are administered and the cell or tissue graft is transplanted only following establishment of chimerism (e.g. immature hematopoietic cells, veto cells an kidney).

According to a specific embodiment, the immature hematopoietic cells may be administered in a single administration, e.g. comprising CD3⁺/CD19⁺-depleted CD34⁺ cells (e.g. when using cryopreserved cells which are thawed on the day of transplantation).

According to a specific embodiment, the immature hematopoietic cells may be administered in two or more administrations, e.g. a first administration comprising CD34⁺ selected cells and a second administration comprising CD3⁺/CD19⁺-depleted cells (e.g. when using fresh cells, i.e. cells which are collected and used within about 1, 2, 3, 4, 5, 6, 7, 8, 9 or 10 days, e.g. within about 1 day). It will be appreciated that these can be administered concomitantly or subsequent to each other (e.g. on the same day or within 1, 2, 3, 4, 5 days of each other).

The non-GVHD inducing cells comprising a Tcm phenotype of some embodiments of the invention may be administered via any method known in the art for cell transplantation, such as but not limited to, cell infusion (e.g. intravenous) or via an intraperitoneal rout.

According to one embodiment, the non-GVHD inducing cells comprising a Tcm phenotype are used as fresh cells.

According to one embodiment, the non-GVHD inducing cells comprising a Tcm phenotype are cryopreserved.

The non-GVHD inducing cells comprising a Tcm phenotype of some embodiments of the invention can be administered to an organism per se, or in a pharmaceutical composition where it is mixed with suitable carriers or excipients.

Likewise, the cell or tissue transplant, e.g. immature hematopoietic cells (e.g. T cell depleted immature hematopoietic cells) of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the Tcm cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

According to one embodiment, the non-GVHD inducing cells comprising a Tcm phenotype are formulated for administration as fresh cells.

According to one embodiment, the non-GVHD inducing cells comprising a Tcm phenotype are formulated for administration as cryopreserved cells.

Pharmaceutical compositions suitable for use in context of some embodiments of the invention include compositions wherein the active ingredients are contained in an amount effective to achieve the intended purpose. More specifically, a therapeutically effective amount means an amount of active ingredients (non-GVHD inducing Tcm cells) efficient for tolerization (i.e. veto effect), anti-disease effect, anti-tumor effect and/or immune reconstitution without inducing GVHD. Since the Tcm cells of the present invention home to the lymph nodes following transplantation, lower amounts of cells (compared to the dose of cells previously used, see for example WO 2001/049243) may be needed to achieve the beneficial effect/s of the cells (e.g. tolerization, anti-disease, anti-tumor effect and/or immune reconstitution). It will be appreciated that lower levels of immunosuppressive drugs (discussed above) may be needed in conjunction with the Tcm cells of the present invention. Likewise, lower levels of non-myeloablative drugs (discussed above) may be needed in conjunction with the Tcm cells of the present invention (such as exclusion of ATG from the pre-transplant conditioning protocol).

Determination of the therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

For example, in case of cell transplantation (e.g. transplantation of megadose T cell depleted immature hematopoietic cells, as discussed hereinbelow) the number of Tcm cells infused to a subject should be more than 1×10⁶ cells per kg ideal body weight. The number of Tcm cells infused to a subject should typically be in the range of 0.01×10⁶ to 20×10⁶ cells per kg ideal body weight, 0.01×10⁶ to 0.5×10⁶ cells per kg ideal body weight, 0.01×10⁶ to 1×10⁶ cells per kg ideal body weight, 0.01×10⁶ to 5×10⁶ cells per kg ideal body weight, 0.1×10⁶ to 0.5×10⁶ cells per kg ideal body weight, 0.1×10⁶ to 1×10⁶ cells per kg ideal body weight, 0.1×10⁶ to 5×10⁶ cells per kg ideal body weight, 0.5×10⁶ to 1×10⁶ cells per kg ideal body weight, 0.5×10⁶ to 1×10⁶ cells per kg ideal body weight, 0.5×10⁶ to 5×10⁶ cells per kg ideal body weight, 1×10⁶ to 5×10⁶ cells per kg ideal body weight, 1×10⁶ to 20×10⁶ cells per kg ideal body weight, 5×10⁶ to 20×10⁶ cells per kg ideal body weight, 10×10⁶ to 15×10⁶ cells per kg ideal body weight, 10×10⁶ to 20×10⁶ cells per kg ideal body weight or 15×10⁶ to 20×10⁶ cells per kg ideal body weight.

According to a specific embodiment, the dose of Tcm cells infused to a subject is about 2.5×10⁶ CD8⁺ cells per kg ideal body weight.

According to a specific embodiment, the dose of Tcm cells infused to a subject is about 5×10⁶ CD8⁺ cells per kg ideal body weight.

According to a specific embodiment, the dose of Tcm cells infused to a subject is about 10×10⁶ CD8⁺ cells per kg ideal body weight.

The term “ideal body weight” as used herein, refers to the measurement used clinically to adjust drug dosing, help estimate renal function and the pharmacokinetics (such as in obese patients).

The formula for estimating ideal body weight in (kg) is as follows:

Males: IBW=50 kg+2.3 kg for each inch over 5 feet.

Females: IBW=45.5 kg+2.3 kg for each inch over 5 feet.

Ideal body weight is discussed in detail in Peterson et al. [Am J Clin Nutr 2016; 103:1197-203], incorporated herein by reference.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide ample levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to one embodiment, the non-GVHD inducing cells comprising a Tcm phenotype of some embodiments of the invention can be manufactured for an “off-the-shelf” product for therapy (e.g. as veto cells for use in therapy as discussed above).

According to one embodiment, the subject is not treated chronically (e.g. for a prolonged period of time, e.g. for more than 8, 9, 10, 12, 14, 21, 25, 30, 45, 60 or 90 days e.g. 10-21 days, e.g. 10 or 14 days) with GVHD prophylaxis post-transplant (e.g. with corticosteroids and/or with immunosuppressive agents).

According to one embodiment, in case of relapse after hematopoietic stem cell transplantation, the subject may be further treated by donor lymphocyte infusions (DLIs). For example, the subject may be administered with graded doses of T-cells as previously described by Dazzi et al [Dazzi, Szydlo et al., Blood, (2000) 96: 2712-6] fully incorporated herein by reference.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Clinical Protocol

### Patient Inclusion Criteria

Age 12-70 years
Patients with a diagnosis either follicular lymphoma (FL), mantle cell lymphoma (MCL), chronic lymphocytic leukemia (CLL), multiple myeloma (MM), Hodgkin's lymphoma (HL), non-hodgkin's lymphoma (NHL), chronic myeloid leukemia (CML), myeloproliferative syndromes (MPD), acute myeloid leukemia (AML) or acute lymphoid leukemia (ALL).
Patients with aplastic anemia and severe immune deficiency or non-malignant bone marrow failure states.
Patients with hematological malignancies must have had persistent or progressive disease despite initial chemotherapy and must have achieved stable disease or a partial or complete response to their most recent chemotherapy.
Availability of a cell donor
Karnofsky performance status≥70%
   Adequate major organ system function as demonstrated by:
   Left ventricular ejection fraction of at least 40%
   Pulmonary function test (PFT) demonstrating an adjusted diffusion capacity of least 50% predicted value for hemoglobin concentration
   Serum creatinine≤1.5 mg/dl.
   SGPT≤200 IU/ml
   Bilirubin<1.5 mg/dl (unless Gilbert's syndrome).
   Negative pregnancy test in women with child bearing potential.

### Patient Exclusion Criteria

HIV seropositive.
Uncontrolled infection or serious medical or psychiatric condition that would limit tolerance to the protocol treatment.
Active CNS malignancy.

### Treatment Plan

There are two collections from the donor, one “unprimed” mononuclear cells (MNC) for the preparation of the Veto Cells and the second collection of mobilized peripheral blood progenitor cells (PBPCs) for preparation of the megadose T cell depleted stem cell transplant. Both collections follow standard of care procedures (SOC) and donors sign SOC consent for donation.

The collection of unprimed (i.e. non-mobilized) peripheral blood mononuclear cells for production of the anti-viral central memory CD8⁺ veto T cells may be done at any time, but generally are done about 7 days before the planned transplant date (i.e. Day 0, i.e. D0). The veto cells are either used fresh at the end of their manufacturing and are to be infused on D+7, or are cryopreserved at the end of their manufacturing.

The collection of the mobilized PBPCs using G-CSF and plerixafor, if clinically necessary, for the megadose transplant can be completed 7 days or more before collection of the PBMCs for production of the of the anti-viral central memory CD8⁺ veto cells. Alternatively, fresh CD34⁺ stem cells can be used wherein collection of the mobilized stem cells can be performed on three consecutive days (day −2, day −1 and day 0) while collection of PBMC for veto cells can begin on day −8. An optimal PBMC cell dose goal of ≥10×10⁶ CD34⁺ cells per kg ideal body weight is procured; a minimum of 5×10⁶ CD34⁺ cells per kg ideal body weight is required which includes an unfractionated aliquot of PBPCs containing approximately 1×10⁶ CD34⁺ cells per kg ideal body weight to be cryopreserved as a backup in case of inadequate veto cell production or graft failure post-transplant or to be available for treatment of graft-failure. If the donor does not tolerate the procedure, or an adequate cell dose cannot be collected, an alternative donor may be used if available.

The mobilized PBPCs are generally collected in 3 days. The first 2 days collections are pooled and CD34⁺ cells selected using the CliniMacs device (Miltenyi), the cells can be used fresh (along with the cells of the third collection) or may be cryopreserved. The third day's collection are depleted of CD3⁺/CD19⁺ cells, the cells can be used fresh (along with the cells of the previous 2 days collections) or may be cryopreserved. The maximum T-cell dose in the entire T-cell depleted stem cell transplant is 2×10⁵ CD3⁺ cells/kg ideal recipient body weight. The back-up fraction of unmanipulated PBPCs are removed from the first day's collection before the CD34⁺ cell selection procedure is performed on the remaining PBPC product.

When using fresh CD34⁺ selected and CD3⁺/CD19⁺-depleted stem cells, these are typically administered on day −1 and 0, respectively. Alternatively, when using cryopreserved CD34⁺ selected and CD3⁺/CD19⁺-depleted stem cells they are typically thawed and infused on day 0.

### Production of Anti-Viral Central Memory CD8⁺ Veto T Cells (Veto Cells)

A detailed protocol for production of veto cells is provided in Example 2, hereinbelow. This section provides a brief description of some embodiments of the invention.

The protocol comprises of three steps:

### Step 1: Preparation of Stimulators (Day −8 to day −5):

Preparation of Donor DC from monocytes in PBMC

Day −8: Approximately 1×10¹⁰ mononuclear cells are collected by leukapheresis and kept overnight.

Day −7: The cells are processed by Ficol separation and thereafter half of the mononuclear cells are used for monocyte isolation by CD14 magnetic beads (Miltenyi magnetic beads sorting system. Thereafter, the CD14⁺ monocytes are differentiated to immature DC with granulocyte macrophage-colony stimulating factor (GM-CSF) and interleukin 4 (IL-4).

In parallel, the CD14⁻ (i.e. CD14 neg) cells are combined with half of the initial mononuclear cells obtained after ficol isolation and stored overnight in 37° C. 5% O2/CO2 in the presence of IL-7. These cells are used for the enrichment of CD8⁺ memory T cells on the next day.

Day −6: DC Maturation by addition of cytokine-cocktail comprising lipopolysaccharide (LPS), Interferon gamma (IFNγ), GM-CSF, and IL-4 for 16 hours of incubation.

Day −5: Mature DCs (mDCs) are harvested, loaded with viral peptide cocktail and irradiated.

Step 2: Preparation of Responders and Co-Culture with Viral Loaded mDC (Day −6 to Day −5):

A) Memory T Cell (CD4⁻CD56⁻CD45⁻) Purification (from Donor PBMC—Responders)

Day −6: Donor CD14⁻ cells combined with half of the mononuclear cells that were incubated overnight with IL-7, are depleted of CD4⁺, CD56⁺, and CD45RA⁺ cells by magnetic beads and stored for an additional night in 37° C. 5% O2/CO2 in the presence of IL-7.

B) Establishment of Responder (Donor) Stimulator (Donor Viral Peptides Loaded DCs) Co-Culture.

Day −5: Purified memory responder T cells (CD4⁻CD56⁻CD45RA⁻) are incubated for 3 days with viral peptide loaded mDCs in the presence of IL-21.

### Step 3: Differentiation and Expansion of Anti-Viral Veto Tcm (Day −5 to +7):

A) Expansion of Anti-Viral Central Memory CD8⁺ Veto T Cells (Tcm)

Days −5 to −2: Initiation of Tcm phenotype with IL-21 only.

Days −2 to +7: Expansions of Tcm cultures by splitting according to glucose level consumption and addition of medium supplemented with the following cytokines: IL-7, IL-15 and IL-21 depending on the observed expansion in culture.

Day +7: Harvest and infusion of cells.

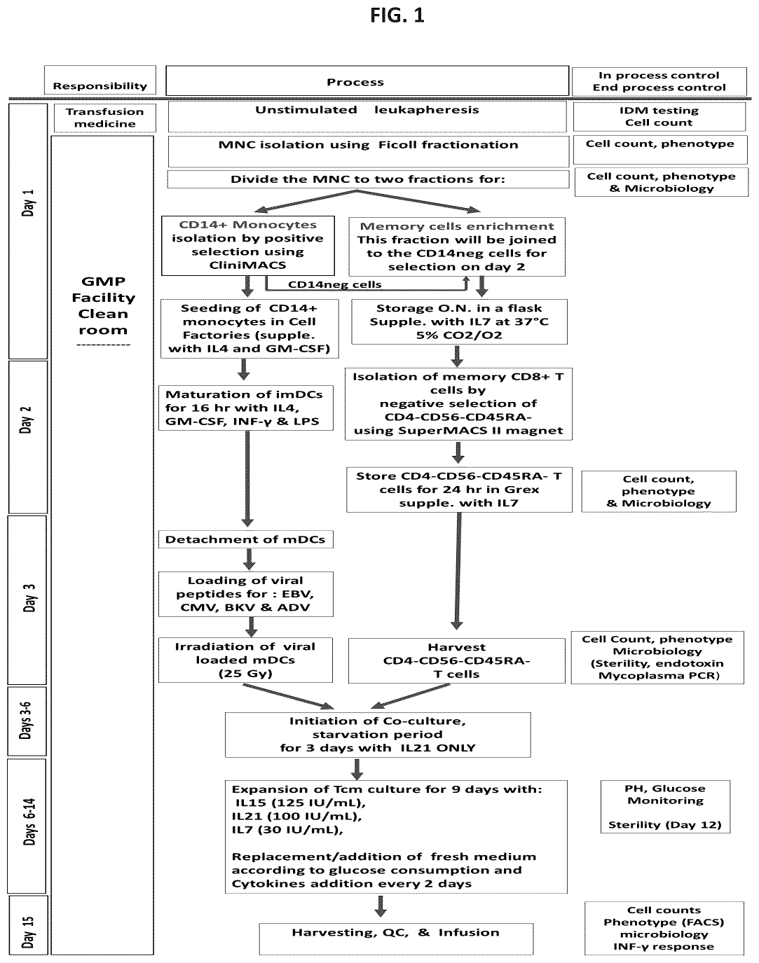

Approximate timeline of Tem production and DC preparation from the intended PBMC donor is shown in FIG. 1. Cells are harvested and infused on day +7 post hematopoietic stem cell transplant.

An aliquot of the anti-viral central memory CD8⁺ veto cells are assessed for release criteria and functional immune studies (as discussed below).

Treatment Plan—Conditioning Regimen

Including Fresh CD34⁺ Cells

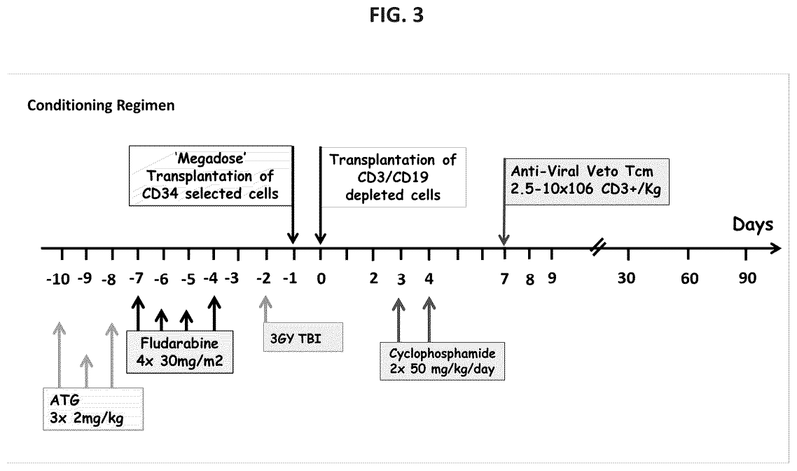

As illustrated in FIG. 3, the conditioning regimen from D−10 to D−2 (i.e. days 10 to 2 before the transplant), includes Anti-thymocyte globulin (ATG) (3 doses from day −10 to day −8), Fludarabine (Flu) (4 doses from day−7 to day −4) and 3 Gy total body irradiation (TBI) (once on day−2). The T-cell depleted stem cell graft is infused on day −1 and day 0. On D+3 and +4 post-transplant (i.e. days 3 and 4 after the transplant), the patient receives cyclophosphamide (CY) followed by the infusion of the veto cells on D+7 post-transplant (i.e. on day 7 after transplant). Patients with B-cell malignancies may receive rituximab 375 mg/m² on day −11.

Day and Treatment:
−11 Admit/Intravenous (IV) hydration/Rituxan 375 mg/m² (for B-cell malignancies)
−10 ATG (Thymoglobulin) 2 mg/kg
−9 ATG (Thymoglobulin) 2 mg/kg
−8 ATG (Thymoglobulin) 2 mg/kg
−7 Fludarabine 30 mg/m²
−6 Fludarabine 30 mg/m²
−5 Fludarabine 30 mg/m²
−4 Fludarabine 30 mg/m²
−3 Rest (no treatment)
−2 TBI 3 Gy in a single fraction.
−1 Infusion of megadose T-cell depleted PBPCs (containing the CD34⁺ selected cells)
0 Infusion of CD3⁻/CD19⁻ depleted cells
+3 Cyclophosphamide (CY) 50 mg/kg/day IV
+4 Cyclophosphamide (CY) 50 mg/kg/day IV
+7 Infusion of Anti-viral veto cells at doses as indicated.

Of note, patients receive a dose of Mesna 10 mg/kg intravenous piggy back (IVPB) just prior to the first dose of cyclophosphamide. This is repeated every 4 hours for a total of 10 doses. Patients also receive ondansetron (or other anti-emetic) prior to each dose of Cyclophosphamide (Cy).

Premedication for the Veto cells does not include corticosteroids.

With Cryopreserved CD34⁺ Cells

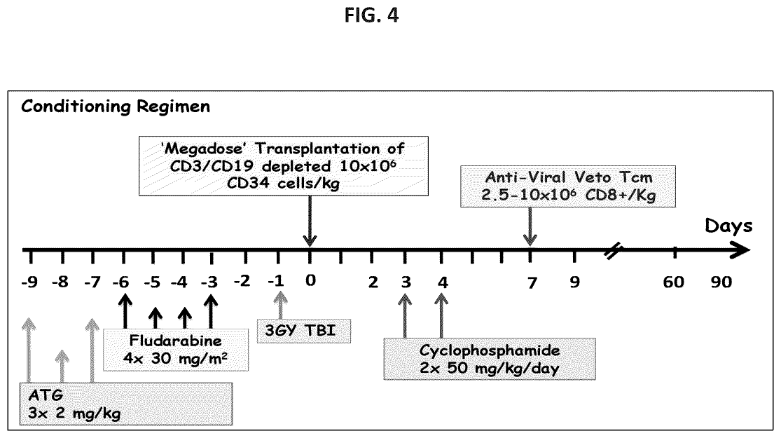

As illustrated in FIG. 4, from D-9 to D-1 (i.e. days 9 to 1 before the transplant), the patient receives Anti-thymocyte globulin (ATG), Fludarabine (Fu) and low dose total body irradiation (TBI) as the preparative regimen. On D+3 and +4 after transplant (i.e. days 3 to 4 after the transplant), the patient receives cyclophosphamide (CY) followed by the infusion of the veto cells on day 7 post-transplant (D+7 after transplant). Patients with B-cell malignancies may receive rituximab 375 mg/m² on day −10.

Day and Treatment:
−10 Admit/Intravenous (IV) hydration/Rituxan 375 mg/m² (for B-cell malignancies)
−9 ATG (Thymoglobulin) 2 mg per kg ideal body weight
−8 ATG (Thymoglobulin) 2 mg per kg ideal body weight
−7 ATG (Thymoglobulin) 2 mg per kg ideal body weight
−6 Fludarabine 30 mg/m²
−5 Fludarabine 30 mg/m²
−4 Fludarabine 30 mg/m²
−3 Fludarabine 30 mg/m²
−2 Rest (no treatment)
−1 TBI 3 Gy in a single fraction.
0 infusion of megadose T cell depleted PBPCs (containing the CD34⁺ selected cells and CD3⁺/CD19⁺-depleted cells)
+3 Cyclophosphamide (CY) 50 mg/kg ideal body weight/day IV
+4 Cyclophosphamide (CY) 50 mg/kg ideal body weight/day IV
+7 infusion of Anti-viral veto cells at doses as indicated.

Of note, patients receive a dose of Mesna 10 mg/kg intravenous piggy back (IVPB) just prior to the first dose of cyclophosphamide. This is repeated every 4 hours for a total of 10 doses. Patients also receive ondansetron (or other anti-emetic) prior to each dose of Cyclophosphamide (Cy).

Premedication for the Veto cells does not include corticosteroids.

Anti-Viral Veto Cell Dose Levels—CD8⁺ Cell Dose Escalation

Dose level 1: 2.5×10⁶ CD8⁺ cells per kg ideal body weight

Dose level 2: 5.0×10⁶ CD8⁺ cells per kg ideal body weight

Dose level 3: 10×10⁶ CD8⁺ cells per kg ideal body weight

No additional post-transplant immunosuppressive therapy is administered as GVHD prophylaxis.

Consideration if Manufactured Cell Product is Less than the Planned Dose Level

Dose level one comprises 2.5×10⁶ cells per kg ideal body weight. Of note, a lower dose is not typically considered acceptable.

For dose level 2, the target dose is 5×10⁶ cells per kg ideal body weight, however, a dose of 2.6 to 5×10⁶ cells per kg ideal body weight is considered adequate and considered dose level 2.

For dose level 3, the target dose is 10×10⁶ cells per kg ideal body weight, however, a dose of 5.1 to 10×10⁶ cells per kg ideal body weight is considered adequate, and considered dose level 3.

Evaluation During Study

Effort is made to adhere to the schedule of events and all protocol requirements. Variations in schedule of events and other protocol requirements that do not affect the rights and safety of the patient are not considered as deviations. Such variations may include laboratory assessments completed outside of schedule and occasional missed required research samples. Missed samples for correlative studies are not constitute protocol deviations.

Evaluation Prior to Transplant (Baseline):

Standard work up for transplant as well as disease assessment is done prior to study entry as part of diagnostic or routine pre-transplant evaluation. The following tests are standard of care pre-transplant tests and not protocol specific. The results are used to determine transplant eligibility and are not repeated prior to the beginning of treatment. If the treatment is delayed for more than 30 days after consenting, the PI or designee should determine which, if any, tests need to be repeated as clinically indicated.
CBC, differential and platelets
Bilirubin, serum creatinine and creatinine clearance, ALT, albumin, electrolytes, LDH, alkaline phosphatase
Infectious diseases panel (hepatitis serology (B, C), HIV, HTLV, I/II, CMV, TPHA screen), toxoplasma serology, Strongyloides serology (if indicated), tuberculosis, IRGA (if indicated)
PT and PTT
ABO and Rh typing
Bone marrow aspiration with cytogenetics and molecular studies if clinically indicated
Chimerism analysis baseline
Pregnancy test in females of childbearing potential
Pulmonary function test with DLCO
Echocardiogram to assess Left ventricular EF and pulmonary artery pressure
Chest X ray and ECG
Serum for donor-specific anti-HLA antibodies (DSA)

The active treatment period for patients treated in this study is from the beginning of the preparative regimen (Day −9) through Day +42 post-transplant. After that, patients have follow-up as clinically indicated through D+100. Thereafter, disease relapse, infections, acute and chronic GVHD and survival data are collected according to the standard follow up of stem cell transplant recipients. After one year, patients are removed from the study. Acute GVHD and Chronic GVHD are scored according to NIH Consensus Criteria.

Standard Post Evaluations

Standard Post Evaluations are per SOC post allogeneic transplant. Bone marrow aspiration and peripheral blood—to evaluate treatment response, engraftment, chimerism and immune reconstitution—are performed monthly for 3 months and as clinically indicated.

Correlative Studies

Immune reconstitution is assessed by flow cytometry immunophenotype panel and antiviral responses by tetramer analysis. Immune tolerance test is performed approximately 3 months post-transplant. Missed samples for correlative studies are not constitute protocol deviations.

The development of acute GVHD is assessed and scored by NIH consensus criteria. Disease relapse or progression is assessed by morphologic relapse of the patient's malignancy. Data on bacterial, viral fungal and parasitic infections is collected. The development of chronic GVHD is described as per NIH Consensus Criteria [Jagasia M H, et al. National Institutes of Health Consensus Development Project on Criteria for Clinical Trials in Chronic Graft-versus-Host Disease: I. The 2014 Diagnosis and Staging Working Group report. Biol Blood Marrow Transplant. (2015) 21:389-401 e381].

TABLE 1A
Table of Assessments

| | Pretreatment | Week 1 | Week 2 | Week 3 | Day 30 | Day 60 | Day 90 | Day 100 |
|---|---|---|---|---|---|---|---|---|
| | (within 30 days of | (+/−1 | (+/−2 | (+/−3 | (+/−5 | (+/−5 | (+/−5 | (+/−14 |
| | study enrollment) | day) | days) | days) | days) | days) | days) | days) |
| Kamofsky Performance | X | | | | | | | |
| Status | | | | | | | | |
| Laboratory Tests¹ | X | | | | | | | |
| Pregnancy Test² | X | | | | | | | |
| CBC | | X | X | X | X | X | X | X |
| Chimerism Analysis | X | X | X | X | X | X | X | X |
| Pulmonary Function | X | | | | | | | |
| Test with DLCO | | | | | | | | |
| ECHO | X | | | | | | | |
| Chest x-ray | X | | | | | | | |
| Bone marrow aspiration³ | X | | | | X³ | X³ | X³ | |
| Correlative Studies⁴ | | | | | X | X | X | |

¹Baseline Laboratory Tests: CBC, differential and platelets, bilirubin, serum creatinine and creatinine clearance, ALT, albumin, electrolytes, LDH, alkaline phosphatase, infectious diseases panel (hepatitis serology (B, C), HIV, HTLV, I/II, CMV, TPHA screen), toxoplasma serology, strongyloides serology (if indicated), tuberculosis IRGA (if indicated), PT and PTT, ABO and Rh typing, serum for donor-specific anti-HLA antibodies (DSA).
²Pregnancy test (urine) in females of childbearing potential.
³If clinically indicated.
⁴Correlative studies (immune reconstitution will be assessed by flow cytometry immunophenotype panel and antiviral responses by tetramer analysis), immune tolerance test will be performed as feasible by Dr. Yair Reisner's lab approximately monthly for 3 months. Missed samples for correlative studies will not constitute protocol deviations.

Supportive Care
No post-transplant growth factors
No post-transplant immunosuppressive therapy is administered as GVHD prophylaxis.
Other aspects of supportive care are administered as clinically indicated (e.g. support by G-CSF in cases in which engraftment is slow, or treatment of GVHD by e.g. methylprednisolone, tacrolimus or other immunosuppressive medications). Concomitant medications administered to the patient during the study are documented in the patient's primary medical record

Contingencies for Inadequate Anti-Viral Veto Cell Production

If the veto cells meet release criteria, but the cell dose is less than the target cell dose level: the cells are infused if the dose is ≥2.5×10⁶ CD8⁺ cells per kg ideal body weight (dose level 1) In this case, the patients are evaluated in their assigned dose level by the intention to treat principle.

If the veto cells dose is less than 2.5×10⁶ CD8⁺ cells per kg ideal body weight, or if the veto cells do not meet release criteria, these cells are not infused. Instead, the backup PBPCs plus the T cell depleted PBPCs are infused to reconstitute a “standard PBPC transplant,” and the patient receives a standard of care preparative regimen, with tacrolimus and mycophenolate as standard GVHD prophylaxis.

Donor Lymphocyte Infusion (DLI) for Persistent or Progressive Disease

If GVHD is not present, patients who have achieved engraftment and have ≥5% myeloid donor cells and who have progressive disease at any time or persistent disease at ≥3 months post-transplant may receive donor lymphocyte infusion as standard of care or alternative therapy.

Treatment of Graft Failure

This is a non-myeloablative regimen. It is anticipated that autologous hematopoietic recovery will likely occur if graft rejection occurs. Engraftment is defined as detection of donor cells by chimerism analysis and recovery of an absolute neutrophil count, ANC>0.5×10⁹/L. Patients who fail to have donor cell engraftment and hematopoietic recovery (ANC>0.5×10⁹/L after day 28) or who achieve recovery but subsequently have ANC fall to <0.5×10⁹/L with at least 3 consecutive determinations over a 5 day period with <10% cellularity on bone marrow biopsy and no evidence of donor chimerism (not associated with reversible infection, myelosuppressive drug treatment or disease progression) are defined as graft failure and considered treatment failures and taken off study. These patients are considered for re-transplantation as clinically indicated and may use the backup unmodified PBPCs.

Interventions for GVHD Occurring after Transplant

The following are guidelines for management of acute GVHD. This is modified as clinically indicated by the patient's attending physician.

Acute GVHD
a) Grade I—monitor without medication (Rx) for 1 week or topical steroids only.
b) If grade II-IV occurs, treat with methylprednisolone 1-2 mg/kg ideal body weight/day.
c) If, after addition of methylprednisolone, grade≥II persists for 48 hrs or progresses, tacrolimus or other immunosuppressive medications may be added as clinically indicated or on active GVHD treatment protocols.

Patients with grade 3-4 steroid resistant acute GVHD are considered treatment failures and may receive other immunosuppressive treatments as clinically indicated.

Statistical Considerations

1. Preliminaries. This is a phase I-II trial to determine the optimal dose of donor-derived veto cells given to patients with hematologic malignancies, including NHL, AML, CLL, or MM, who previously have relapsed or have been declared refractory to initial therapy, and subsequently have achieved partial or complete remission. Each patient first receives an allogeneic stem cell transplant (allosct) from a haplo-identical donor, with conditioning regimen comprising of ATG, fludarabine and TBI, with double pulsed cyclophosphamide given as GVHD prophylaxis on days 3 and 4 following allosct, followed by veto cells given on day 7 post allosct. The veto cell dose of each patient cohort is chosen adaptively using the phase I-II design.

2. Dose-Finding Design

Goal: The primary scientific goal of the trial is to determine an optimal veto cell dose among the three levels 2.5×10⁶, 5×10⁶, and 10⁷ CD8⁺ cells per kilogram ideal body weight, hereafter referred to and coded numerically in the dose-outcome model as doses 1, 2, 3 (see Table 1B, below). Dose-finding are done using the sequentially adaptive phase I-II EffTox trade-off-based design as previously discussed [Thall P F and Cook J D. Dose-finding based on efficacy-toxicity trade-offs. Biometrics (2004) 60:684-693; Thall P F et al. Using effective sample size for prior calibration in Bayesian phase I-II dose-finding. Clinical Trials. (2014) 11:657-666].

Co-Primary Outcomes: For the purpose of dose-finding, (1) Toxicity is defined as a grade 3 or 4 GVHD, or death from any cause, within 42 days of veto cell infusion; and (2) Efficacy is defined as the patient being alive and engrafted at day 42 post veto cell infusion. A patient who is enrolled in the trial but, for any reason, does not receive veto cells on day 7 post allosct are replaced in the sample.

Dose Acceptability: For the two EffTox dose acceptability rules, the upper limit on the probability of Toxicity is 0.20, the minimum probability of Efficacy 0.80, and posterior probability decision cut-offs 0.10 are used or both acceptability criteria. If the lowest dose level is found to be unacceptable in terms of either high Toxicity or low Efficacy, the trial are terminated and no veto cell dose level are selected.

Maximum Sample Size and Cohort Size: A maximum of 24 patients are treated in up to 8 cohorts of size 3, with the first cohort treated at dose d=2, and doses for all subsequent cohorts chosen adaptively to maximize the Efficacy-Toxicity trade-off computed based on the most recent accumulated data. Accrual are suspended between cohorts, if necessary, to ensure that both 42-day Efficacy and Toxicity outcomes are evaluated for all previously treated patients in order to apply the outcome-adaptive rules.

Prior: The prior on the model parameters was computed using the EffTox program version 4.0.12, from the elicited mean marginal outcome probabilities given on Table 1B, with hyper-variances calibrated to obtain prior effective sample size 1.

TABLE 1B
Elicited prior mean probabilities of Toxicity
and Efficacy for each veto cell dose level

| | 2.5 × 10⁶ veto | 5 × 10⁶ veto | 10 × 10⁶ veto |
|---|---|---|---|
| | cells per kg | cells per kg | cells per kg |
| | ideal body weight | ideal body weight | ideal body weight |
| | d = 1 | d = 2 | d = 3 |
| Pr (Toxicity) | .10 | .20 | .30 |
| Pr (Efficacy) | .70 | .80 | .90 |

Efficacy-Toxicity Trade-Off Contours: The three equivalent trade-off probability pairs used for computing the trade-off contours are (0.30, 0), (0.55, 0.30), and (1, 0.80).

3. Design Operating Characteristics

Operating characteristics (OCs) of the design, under six hypothetical dose-outcome scenarios, are summarized below. The simulations to compute the OCs were carried out using EffTox version 4.0.12, with 2000 replications per scenario, using simulation seed 10502.

TABLE 2A
Operating characteristics of the design

| | Dose Level | | | |
|---|---|---|---|---|
| Scenario | 1 | 2 | 3 | None |

1

| | | | | |
|---|---|---|---|---|
| True pT, pE | .10, .60 | .20, .80 | .40, .85 | — |
| Trade-off Value | 0.31 | 0.47 | 0.30 | — |
| % selected | 10 | 68 | 5 | 17 |
| # Patients Treated | 3.4 | 17.3 | 1.6 | — |

2

| | | | | |
|---|---|---|---|---|
| True pT, pE | .02, .60 | .05, .70 | .10, .80 | — |
| Trade-off Value | 0.41 | 0.51 | 0.60 | — |
| % selected | 6 | 29 | 58 | 7 |
| # Patients Treated | 1.3 | 12.1 | 9.9 | — |

3

| | | | | |
|---|---|---|---|---|
| True pT, pE | .02, .60 | .05, .70 | .10, .90 | — |
| Trade-off Value | 0.41 | 0.51 | 0.74 | — |
| % selected | 2 | 29 | 67 | 2 |
| # Patients Treated | 0.6 | 12.1 | 11.0 | — |

4

| | | | | |
|---|---|---|---|---|
| True pT, pE | .15, .80 | .35, .82 | .45, .85 | — |
| Trade-off Value | 0.54 | 0.32 | 0.24 | — |
| % selected | 62 | 20 | 1 | 18 |
| # Patients Treated | 11.3 | 10.3 | 0.4 | — |

5

| | | | | |
|---|---|---|---|---|
| True pT, pE | .40, .65 | .45, .75 | .50, .80 | — |
| Trade-off Value | 0.02 | 0.10 | 0.10 | — |
| % selected | 15 | 4 | 1 | 80 |
| # Patients Treated | 6.6 | 6.9 | 0.5 | — |

6

| | | | | |
|---|---|---|---|---|
| True pT, pE | .02, .40 | .05, .45 | .10, .50 | — |
| Trade-off Value | 0.12 | 0.16 | 0.17 | — |
| % selected | 8 | 1 | 9 | 81 |
| # Patients Treated | 3.1 | 5.2 | 6.9 | — |

4. Implementation

The EffTox design are implemented using the MD Anderson Cancer Center (MDACC) Department of Biostatistics Clinical Trial Conduct website www(dot)biostatistics(dot)mdanderson (dot)org/ClinicalTrialConduct/. The study biostatistician Peter Thall or a designated Statistical Analyst in the Biostatistics Department should be consulted if necessary during trial conduct.

5. Secondary Outcomes and Data Analyses

Secondary outcomes include infection, progression-free survival (PFS) time, overall survival (OS) time, cytomegalovirus, and immune reconstitution, with a 1-year follow up period for each patient. Unadjusted distributions of time-to-event outcomes are estimated using the method of Kaplan and Meier [Kaplan, E L and Meier, P: Nonparametric estimator from incomplete observations. J. American Statistical Association (1958) 53:457-481] and their relationship to prognostic covariates and veto cell dose level are evaluated by Bayesian piecewise exponential survival regression [Ibrahim J G, Chen M-H, Sinha D. Bayesian Survival Analysis. Springer, NY (2001)].

Data and Protocol Management

Protocol Compliance

Patients are monitored for toxicity and engraftment weekly during the initial 28 days and as clinically indicated by the clinical trial Investigator and/or his designee(s). The Study Chairman are the final arbiter of toxicity should a difference of opinion exist.

Criteria for Removal from the Study

Patients are taken off study for any of the following:
1) Withdrawal of patient consent to participate.
2) Inadequate veto cell product for infusion.
3) Progression of disease.
4) Development of graft failure requiring second transplantation.
5) One year after treatment.
6) An increasing or unexpected pattern of toxicity observed deemed unacceptable by the study chairman.
7) Investigator judgment when the well-being and best interest of the patient is compromised.
8) Noncompliance considered by the study chairman to compromise evaluation of the study endpoints
9) Death

### Reporting Requirements

Adverse Event Definition

An adverse event is the appearance or worsening of any undesirable sign, symptom, or medical condition occurring after starting the study drug even if the event is not considered to be related to study drug. Medical conditions/diseases present before starting study drug are only considered adverse events if they worsen after starting study drug. Abnormal laboratory values or test results constitute adverse events only if they induce clinical signs or symptoms, are considered clinically significant, or require therapy.

Assessment of Adverse Events

The Investigator or physician designee is responsible for verifying and providing source documentation for all adverse events and assigning the attribution for each event for all subjects enrolled on the trial.

Adverse events and protocol specific data are entered into Redcap. Redcap is used as the electronic case report form for this protocol.

Severity of the Adverse Events (AEs)

All grades of AEs related to Anti-Veto cell infusion are collected.

General Grading:

Grade 1: Mild: discomfort present with no disruption of daily activity, no treatment required beyond prophylaxis.

Grade 2: Moderate: discomfort present with some disruption of daily activity, require treatment.

Grade 3: Severe: discomfort that interrupts normal daily activity, not responding to first line treatment.

Grade 4: Life Threatening: discomfort that represents immediate risk of death

TABLE 2B
Recommended Adverse Event Recording Guidelines
Recommended Adverse Event Recording Guidelines

| Attribution | Grade 1 | Grade 2 | Grade 3 | Grade 4 | Grade 5 |
|---|---|---|---|---|---|
| Unrelated | Phase I | Phase I | Phase I | Phase I | Phase I |
| | | | Phase II | Phase II | Phase II |
| | | | | Phase III | Phase III |
| Unlikely | Phase I | Phase I | Phase I | Phase I | Phase I |
| | | | Phase II | Phase II | Phase II |
| | | | | Phase III | Phase III |
| Possible | Phase I | Phase I | Phase I | Phase I | Phase I |
| | Phase II | Phase II | Phase II | Phase II | Phase II |
| | | Phase III | Phase III | Phase III | Phase III |
| Probable | Phase I | Phase I | Phase I | Phase I | Phase I |
| | Phase II | Phase II | Phase II | Phase II | Phase II |
| | | Phase III | Phase III | Phase III | Phase III |
| Definitive | Phase I | Phase I | Phase I | Phase I | Phase I |
| | Phase II | Phase II | Phase II | Phase II | Phase II |
| | | Phase III | Phase III | Phase III | Phase III |

Causality Assessment

The investigational component of the treatment plan of this study is the infusion of Veto Cells.

Adverse events related to the infusion of the Veto Cells. Infusion reactions could occur. No other toxicities are expected.

Therefore, events known to be caused by Veto cell infusion and its direct consequences are assessed as definitely related when assessing the causality.

When the relationship of the adverse event cannot be ruled out between the Veto cell infusion and the transplant conditioning, the event are scored as probably or possible related.

Events known to be related to drugs used as chemotherapy as well as to drugs used as supportive treatment are scored as unrelated to the Anti-Veto Cell infusion.

The principal investigator is the final arbiter in determining the causality assessment.

Adverse Events Considered Serious

For the purpose of this study, abnormal laboratory findings considered associated to the original disease as well as isolated changes in laboratory parameters such as electrolyte magnesium and metabolic imbalances, uric acid changes, elevations of GPT, GOT, LDH and alkaline phosphatase are not considered adverse events and are not be collected in the database.

Serious Adverse Event Reporting (SAE)

An adverse event or suspected adverse reaction is considered “serious” if, in the view of either the investigator or the sponsor, it results in any of the following outcomes:
Death
A life-threatening adverse drug experience—any adverse experience that places the patient, in the view of the initial reporter, at immediate risk of death from the adverse experience as it occurred. It does not include an adverse experience that, had it occurred in a more severe form, might have caused death.
Inpatient hospitalization or prolongation of existing hospitalization
A persistent or significant incapacity or substantial disruption of the ability to conduct normal life functions.
A congenital anomaly/birth defect.
Important medical events that may not result in death, be life-threatening, or require hospitalization may be considered a serious adverse drug experience when, based upon appropriate medical judgment, they may jeopardize the patient or subject and may require medical or surgical intervention to prevent one of the outcomes listed in this definition.

Examples of such medical events include allergic bronchospasm requiring intensive treatment in an emergency room or at home, blood dyscrasias or convulsions that do not result in inpatient hospitalization, or the development of drug dependency or drug abuse (21 CFR 312.32).
Important medical events as defined above, may also be considered serious adverse events. Any important medical event can and should be reported as an SAE if deemed appropriate by the Principal Investigator or the IND Sponsor, IND Office.
All events occurring during the conduct of a protocol and meeting the definition of a SAE must be reported to the IRB in accordance with the timeframes and procedures outlined in “The University of Texas M. D. Anderson Cancer Center Institutional Review Board Policy for Investigators on Reporting Unanticipated Adverse Events for Drugs and Devices”. Unless stated otherwise in the protocol, all SAEs, expected or unexpected, must be reported to the IND Office, regardless of attribution (within 5 working days of knowledge of the event).
All life-threatening or fatal events, that are unexpected, and related to the study drug, must have a written report submitted within 24 hours (next working day) of knowledge of the event to the Safety Project Manager in the IND Office.
Unless otherwise noted, the electronic SAE application (eSAE) are utilized for safety reporting to the IND Office and MDACC IRB.
Serious adverse events are captured from the time of the first protocol-specific intervention, until 30 days after infusion of anti-Veto cells, unless the participant withdraws consent. Serious adverse events must be followed until clinical recovery is complete and laboratory tests have returned to baseline, progression of the event has stabilized, or there has been acceptable resolution of the event.
Additionally, any serious adverse events that occur after the 30 day time period that are related to the study treatment must be reported to the IND Office.

### Background Drug Information

Cyclophosphamide

Stability and Storage Requirements:
Prior to mixing: Room temperature
After mixing: Stable for 24 hours at room temperature and 6 days when refrigerated.

Usual Dosage Range: Up to 2000 mg/m² as a single dose, repeated every 3 weeks. Smaller doses may be given more frequently. Doses of up to 1.5 gm/m²/d×4 may be used in conjunction with bone marrow or peripheral blood progenitor cell transplant.

Known Side Effects and Toxicities: Myelosuppression (leukopenia greater than thrombocytopenia), hemorrhagic cystitis, nausea, vomiting, alopecia, and rare amenorrhea and azoospermia.

Special Precautions: Adequate hydration with 2-3 liters of fluid daily with copius urine output can prevent cystitis. Due to significant renal excretion, dose reductions must be made in patients with renal insufficiency.

Mechanism of Action: Cyclophosphamide is considered a classical bifunctional alkylating agent, with the predominant alkylation reaction occurring at the 7 nitrogen of guanine.

Cyclophosphamide must be activated by liver microsomal enzymes in order to damage the DNA molecule. Although active throughout the cell cycle, the agent is most active during the S phase.

Human Pharmacology: Cyclophosphamide can be given orally or intravenously, although oral absorption is incomplete (30-60% of a dose is recoverable in the stool). Maximum plasma levels are achieved within one hour from an oral dose. Plasma T-1/2 is 4-6.5 hours. Approximately 60% of an intravenous dose is recovered in the urine within 24 hours, requiring dosage adjustments in patients with renal insufficiency. The drug is activated and subsequently deactivated by liver microsomal enzymes.

Fludarabine (FU)

Synonyms (Trade names, etc.): Fludara

Therapeutic Classification: fluorinated nucleoside analog

Each vial contains 50 mg lyophilized drug, to be reconstituted with 2 ml sterile water to a solution that is 25 mg/ml for IV administration.

Pharmaceutical Data: Intravenous Powder for Solution: 50 mg

Solution Preparation: mix each vial with 2 ml sterile pyrogen-free water to a clear solution, which is 25 mg/ml for IV administration only. Reconstituted solution should be used within 8 hours.
Use proper procedures for handling and disposal of chemotherapy

Mechanism of Action: After phosphorylation to fluoro-ara-ATP the drug appears to incorporate into DNA and inhibit DNA polymerase alpha, ribonucleotide reductase and DNA primase, thus inhibiting DNA synthesis.

Human Safety and Pharmacology: The half-life of the activated compound is approximately 10 hours, but the pharmacology is incompletely understood. Excretion is impaired in patients with impaired renal function.

Anti-Thymocyte Globulin (ATG) (Thymoglobulin)

Therapeutic Classification: Immunosuppressant

Mechanism of Action: ATG is a rabbit anti-thymocyte globulin. It is a purified, sterile, primarily monomeric IgG fraction of hyperimmune serum of selective immunosuppressant. It is believed to act by modifying the number and function of lymphocytes.

Pharmaceutical Data: ATG is diluted in 0.9% NaCl. ATG is given intravenously as a 4 hour infusion into a central vein. It is a colorless to slightly opalescent protein solution. ATG should not be diluted in dextrose or low salt fluids, as this may cause precipitation. Infusion with acidic solution may cause physical instabilities. Although no further incompatibilities or drug interactions are known, it is recommended to administer ATG alone.

Stability and Storage Requirements: Commercially available. Solution for infusion contains 1 mg of ATG/2 ml. It should be stored in a refrigerator at 2° C. to 8° C. (do not freeze). Diluted ATG is stable for 24 hours.

Known Side Effects: ATG may cause cytopenia of any cell line, chills, fever, or edema. A serum-sickness like syndrome may also develop. Adverse reactions including rash, pruritus, urticaria, wheal and flare reactions, bronchospasm bleeding and anaphylactic shock have been reported. Resuscitation equipment should be available during administration. After an anaphylactic reaction, infusion should not be resumed.

Mesna

Synonyms (Trade Names, etc.): Mesnex

Therapeutic Classification: Hemorrhagic cystitis inhibitor, Antineoplastic Agent

Pharmaceutical Data: 100 mg/ml solution for injection, 400 mg Oral Tablet

Usual Dosage Range:
Cyclophosphamide adverse reaction, High dose; Prophylaxis—Hemorrhagic cystitis; Prophylaxis: 40% IV (W/W) of cyclophosphamide dose at 0, 3, 6, 9 hrs and IV fluids.
Cyclophosphamide adverse reaction, Low dose; Prophylaxis—Hemorrhagic cystitis; Prophylaxis: 20 mg/kg ideal body weight ORALLY every 3-4 hrs.
Hemorrhagic cystitis; Prophylaxis—Ifosfamide adverse reaction; Prophylaxis: bolus IV regimen, IV bolus 20% of ifosfamide dose (w/w) at 0, 4, 8 hrs.
Hemorrhagic cystitis; Prophylaxis—Ifosfamide adverse reaction; Prophylaxis: oral tablet regimen, IV bolus 20% of ifosfamide dose (w/w) at time of infusion, tablets 40% of ifosfamide dose at 2, 6 hrs.
Hemorrhagic cystitis; Prophylaxis—Ifosfamide adverse reaction; Prophylaxis: continuous IV regimen (non-FDA approved regimen), bolus dose (20% total ifosfamide dose), then 40% of ifosfamide CIV.

Known Side Effects and Toxicities: Common adverse effects: fatigue, headache, nausea, vomiting, and diarrhea (occur with higher doses (80 mg/kg ideal body weight)). Hypotension can occur.

Special Precautions: IV mesna should continue 12-24 hr after the completion of ifosfamide or cyclophosphamide.

Contraindications: hypersensitivity to mesna/other thiol compounds.

Drug Interactions: warfarin

### Example 2

### Production of Anti-Viral Central Memory CD8⁺ Veto Cells

### Manufacturing and Characterization Information of the Cell Preparations

Cell Type and Derivation

This protocol requires the collection of two separate stem cells preparations. One type of stem cell preparation is from a mobilized haploidentical donor, part of which is selected for CD34⁺ cells, and the other part depleted of CD3⁺/CD19⁺ cells using the Miltenyi CliniMACS® device and cryopreserved. The second type of preparation is an allogeneic non-mobilized peripheral blood mononuclear (PBMC) cells for the production of the anti-viral central memory CD8⁺ veto T cells. The PBMC are used to generate dendritic cells and memory responder T cells (CD4⁻CD56⁻CD45RA⁻), which are then co-cultured to stimulate production of anti-viral central memory CD8⁺ veto cells (Tcm). The Tcm cells are then expanded, harvested and infused. This preparation is used in the manufacture and infusion of Tcm cells, to achieve engraftment without GVHD after the infusion of T cell depleted megadose haploidentical donor from CD34⁺ enriched cells.

Manufacturing Laboratory

Cell preparations of some embodiments of the invention are processed in the Department of Stem Cell Transplantation and Cellular Therapy (SCTCT) Cell Therapy Laboratory (CTL) at MDACC. This facility contains both a Cell Processing Laboratory for minimally manipulated cell preparations and Classified Suites (Class ISO 7) for more than minimally manipulated cell preparations. The cell preparation of some embodiments of the invention is manufactured primarily in the Class ISO 7 suites. The CTL also includes a Flow Cytometry and Quality Control Laboratories for the support of clinical trials, research, and development.

Relevant Accreditations (FACT, CAP, CLIA)

The SCTCT CTL is registered with the FDA (FEI #0001670014) and is accredited by the Foundation of Accreditation of Cellular Therapy (FACT), the College of American Pathologists (CAP), and holds a Clinical Laboratory Improvement Amendments (CLIA) Certificate for Accreditation issued by the Centers for Medicare and Medicaid Services (CMS).

Starting Material

PBMC: Two separate cell type preparations are required for this protocol. One preparation comprises an allogeneic non-mobilized peripheral blood mononuclear cells for the production of the anti-viral central memory CD8⁺ veto T cells (Tcm) while the other is from a mobilized haploidentical peripheral blood preparation. Both preparations are collected via leukapheresis.

The non-mobilized (unprimed) peripheral blood mononuclear cells for production of the Tcm cells are collected about 7 days before the planned transplant date (Day 0). The target leukapheresis mononuclear cells yield is of 1×10¹⁰. The Tcm cells are infused on D+7 after infusion of the CD34⁺ Enriched Preparation.

The mobilized peripheral blood cells are collected over 3 days. The Day 1 and Day 2 collections are CD34⁺ selected and cryopreserved. The third day's collection is depleted of CD3⁺/CD19⁺ cells and cryopreserved. Multiple collections can be pooled prior to the enrichment procedure. A back up fraction of unmodified PBMC containing 2×10⁶ CD34⁺ cells per kg ideal body weight is taken from the day 1 collection and cryopreserved.

Process Description

MDACC donors are first assessed for suitability including eligibility screening and physical examination. Donors are then consented and scheduled for collection. Target cell numbers are protocol specific and multiple collections may be required. After the collection procedure is complete, cells are transported from the harvesting facility to the CTL Laboratory by CTL staff in plastic coolers to protect the cells from temperature fluctuations and physical damage. Preparations are then logged at the CTL.

Infectious Disease Testing and Prevention of Cross-Contamination

Donor Eligibility

All donors are screened and tested according to the below. Donor must be tested within 7 days for Therapeutic Cells (PBMC).

ZIKA Virus Donor Screening:

Donors are considered ineligible if they have any of the following risk factors:

1. Medical diagnosis of ZIKV infection in the past 6 months.

2. Residence in, or travel to, an area with active ZIKV transmission within the past 6 months.

3. Sex within the past 6 months with a male who is known to have either of the risk factors listed in items 1 or 2, above.

Medical History

MDACC donor and patient medical histories are determined in the SCTCT Clinic by the Attending Physician or their designee(s).

List of Testing

All tests are performed by CLIA-certified laboratories using FDA-approved tests where available. Infectious disease testing includes Anti HIV-1/HIV-2 (DHIV/1/2), Hepatitis C Virus Antibody (DHCVAB), Anti HTLV I/HTLVII (DHTLV I/II), Hepatitis B Antigen (DHBSAG), Hepatitis B Core Antibody (DHBCAB), West Nile Virus-NAT testing (WNV by Nucleic Acid Testing), Serology test for syphilis (Donor TP-HA or RPR), Anti CMV (Donor CMV Antibody), Chagas Disease, HIV, HCV antigen testing by Nucleic Acid Testing (NAT). Of note: The NAT HIV/HCV testing is a combination testing. If result is reactive, discriminatory testing is performed.

Donor Eligibility Undetermined

All cell preparations according to one embodiment obtained from donors where eligibility has not been determined must be stored in quarantine until eligibility has been determined. Cell preparations in quarantine are clearly labeled to prevent release. If cell preparations must be released prior to eligibility determination then urgent medical need must be documented. These cell preparations must be labeled “NOT EVALUATED FOR INFECTIOUS SUBSTANCES” and “WARNING: Advise patient of communicable disease risks”.

Release of Cell Preparations from Ineligible Donor

The release of cell preparations of one embodiment from an ineligible donor requires the documentation of urgent medical need and the consent of the patient. Cell preparations are labeled with a “Biohazard” Label including “WARNING: Advise patient of communicable disease risks”. The screening and test results must accompany the cell preparations and be provided to the infusing physician.

Infusion of Cell Preparations with Positive IDM Results

The infusion of a cell preparation according to one embodiment with a positive IDM result requires the documentation of urgent medical need and the consent of the patient. Cell preparations are labeled with a “Biohazard” Label including “WARNING: Advise patient of communicable disease risks”. Screening and test results must accompany the cell preparation and be provided to the infusing physician. Prophylactic treatment and additional patient monitoring may be necessary.

### Cell Processing

### Generation of the Anti-viral Central Memory CD8⁺ Veto Cells (Tcm Cells)

Procedure Overview

The collection of unprimed peripheral blood mononuclear cells for production of the anti-viral central memory CD8⁺ veto T cells are collected 7 days before the planned infusion date (Day 0). The veto cells are infused on D+7 post megadose T Cell Depleted Cells.

As illustrated in FIG. 1:

Day 1: MNC Isolation and DC Maturation

PBMC Isolation and Thrombowash:

The non-mobilized leukapheresis cell preparation of one embodiment is diluted at 1:2 with Dulbecco's Phosphate-Buffered Saline (DPBS) without Calcium and Magnesium supplemented with 0.5% of Human Serum Albumin, 25% (HSA). Samples for cell count, Trypan Blue (TB) viability and sterility are obtained prior to ficoll process. The MNC are isolated by ficoll density gradient separation. After ficoll, the MNC cell preparation of one embodiment is platelet washed (thrombowash) twice by manual centrifugation prior to CD14⁺ isolation and resuspended with Wash Buffer. Samples for cell counts and TB viability are removed for QC testing.

Following the Post-Ficoll Thrombowash, the cells are divided into two equal fractions and diluted up to approximately 500 mL each. One half are processed for the Dendritic Cell (DC) isolation by CD14⁺ Selection and the other half (Fraction I) are kept overnight for the CD8⁺ Memory T Cell Enrichment process the following day.

Fraction I is centrifuged and resuspended at a concentration of 30×10⁶ cells/ml in T Cell Growth Media (Click's Media with advanced RPMI 1640 supplemented with 1:100 Glutamaxe and 5% Human AB Serum) along with IL-7 (30 IU/mL) Samples for sterility testing are removed. Fraction I is then plated onto tissue culture flasks and incubated overnight at 37° C., 5% CO₂.

DC Isolation—CD14⁺ Monocyte Isolation

The DC isolation fraction is centrifuged and resuspended in 50 ml of Magnetic Bead Buffer (Dulbecco's Phosphate-Buffered Saline (DPBS) without Calcium and Magnesium supplemented with 0.6% ACD-A and 0.5% of 25% HAS). Samples for cell count and TB viability are removed.

The DC Isolation MNC fraction is incubated with the CD14 Reagent (CD14 monoclonal antibodies conjugated to super-paramagnetic iron dextran particles). The cells are then washed with Magnetic Beads buffer to remove excess reagent. Samples for cell count, TB viability and immunophenotyping are removed. Following the wash, CD14 labeled cells are processed on the SuperMACS™ II using the XS Separation Column per established SOP. The magnetically labeled cells (CD14⁺) are retained by the column and the CD14 negative cells are removed. The CD14⁺ cells are then released from the column and collected. The CD14⁺ fraction is washed and resuspended in DCs medium (CellGro/1% HSA), while CD14⁻ fraction is washed and resuspended in T cell growth medium. Samples from each fraction are removed for cell count, TB viability and immunophenotyping. The CD14⁺ enriched cell preparation of one embodiment is then continued to the DC Maturation step, while the CD14 negative cell concentration is adjusted at 30×10⁶ cells/ml in T Cell Growth Media along with IL-7 (30 IU/mL). Samples for sterility testing are removed. The CD14 Negative Fraction is then plated onto tissue culture flasks and incubated overnight at 37° C., 5% CO₂. These cells are combined on the next day with Fraction I for the CD8⁺ Memory T Cell Enrichment process.

DC Maturation—CD14⁺ Monocyte Adherence

The CD14⁺ enriched cell preparation of one embodiment is resuspended at a cell concentration of 3×10⁶ cells/ml in DC Medium (CellGro/1% HSA) supplemented with IL-4 (1000 IU/mL) and GM-CSF (2000 IU/mL). Samples for sterility testing are removed. The cell suspension is then seeded in Cell Factory plates and incubated overnight for 16-24 hours in at 37° C., 5% CO₂.

Day 2: Isolation of Memory Cell and DC Maturation Induction

DC Maturation Induction

After the 24 hour incubation of the CD14⁺ Enriched cells, cytokines: IL-4 (1000 IU/mL), GM-CSF (2000 IU/mL), LPS (40 ng/mL), and IFN-γ (200 IU/mL) are added to the Cell Factories to induce maturation of the DCs. The cells are then incubated with the cytokines at 37° C., 5% CO₂ for 16 hours (+/−2 hours).

Isolation of Memory Cell

After the overnight storage, the cells in Fraction I and in the CD14 Negative fraction are harvested and combined. Once combined (T Cell Isolation TNC Fraction), the cells are centrifuged and resuspended in CliniMACS®/0.5% HSA Buffer to a minimum of 1:2 ratio. Platelet depletion (thrombowash) is performed by centrifugation and resuspension of the cells pellet in CliniMACS®/0.5% HSA. A sample for cell count and TB viability is removed.

The post-platelet depleted cell preparation of one embodiment is incubated with IVIg for 10-15 minutes. After the initial incubation the CD4⁺, CD56⁺ and CD45RA⁺ reagents (CD4⁺, CD56⁺ and CD45RA⁺ antibodies conjugated to super-paramagnetic particles) are added to the cell preparation and incubated for 30 minutes on an orbital rotator. At the end of the incubation the cells are washed by centrifugation and the cell pellet resuspended in CliniMACS®/0.5% HSA buffer to remove excess reagent. Cell count and immunophenotyping samples are removed. The CD4⁺/CD56⁺/CD45RA⁺ labeled cells are then processed on the CliniMACS® using the depletion tubing set and the depletion program. In this case, the magnetically labeled cells (CD4⁺, CD56⁺ and CD45RA⁺) are retained by the column and CD4, CD56 and CD45RA negative cells pass through the column and are collected as a CD4⁻CD56⁻CD45RA⁻ Depleted Fraction (Fraction II). Fraction II is washed and resuspended in T cell Growth Medium. Samples from each fraction are removed for cell count, TB viability and immunophenotyping. Once the volume and TNC of each fraction is determined, the positive fraction is discarded and the negative fraction (Fraction II) is further processed.

Fraction II cell concentration is adjusted at 2×10⁶ cells/ml in T Cell Growth Media along with IL-7 (30 IU/mL). Samples for sterility testing are removed. Fraction II is seeded in G-Rex®100 and incubated at 37° C., 5% with CO₂ for 24 hours (+/−2 hours).

Limitations:

The maximum TNC to process with one reagent kit and tubing set is 200×10⁶ TNC/mL unless higher number is approved by the Laboratory Director or designee. The maximum load volume for the CliniMACS® instrument is 300 ml.

Day 3: Mature DC Harvest and Viral Peptide Loading and Co-Culture

Mature DC (mDC) Harvest

After the 16 hour incubation, the supernatant is removed and the Cell Factories are gently washed with warm Magnetic Bead Buffer. The washing buffer and any non-adherent cells are removed and added to the mDC supernatant. Samples from the mDC supernatant and wash buffer are removed for cell count, TB viability and immunophenotyping.

The adherent mDC are detached and harvested from the Cell Factories by adding ice-cold Magnetic Bead buffer and left resting for 30 minutes on frozen gel packs. After the 30 minutes, the mDC are harvested and the Cell Factories are washed once with ice-cold Magnet bead buffer. The Cell Factories are inspected microscopically to determine whether all the mDCs are removed. If it is observed that not all the mDCs are removed the wash process is repeated. The harvested (adherent) mDCs suspension is then centrifuged, washed and resuspended in Magnetic Bead Buffer. Samples from the adherent mDCs are removed for cell count, TB. After the mDCs volume and TNC is determined the cell concentration is adjusted to 1×10⁷ cells/ml for peptide loading. In-process samples for immunophenotyping are removed. Once the volume and TNC of each fraction is determined the mDC supernatant fraction is discarded and the mDCs cell preparation of one embodiment is further processed.

Loading of Viral Peptides

After mDCs are harvested, the calculated peptivators (AdV5 Hexon, HCMV pp65, EBV select and BKV LT) are added to the cells and are incubated for 1 hour at 37° C., 5% CO₂. Following the incubation the viral peptide loaded mDCs are washed and centrifuged with Magnetic Bead Buffer. Then the viral peptide loaded mDCs are resuspended with T cell Growth Medium and irradiated with 30-25 Gy via X-Ray source.

Once irradiation is complete the viral peptide loaded mDCs are washed, centrifuged and resuspended in T Cell Growth Medium. Samples are removed for cell count, TB viability, immunophenotyping and sterility.

Co-Culture Preparation and Starvation Phase

Following the 24 hour incubation of the Fraction II (CD4⁻CD56⁻CD45RA⁻), the cells are retrieved from the incubator, centrifuged and resuspended in T Cell Growth Medium. Samples for cell count, TB viability, immunophenotyping and sterility are removed. The cells from Fraction II (CD4⁻CD56⁻CD45RA⁻), are then washed and seeded in G-Rex®100 at a concentration of 1×10⁶ cells/ml (100 ml/G-Rex®100) together with the viral peptide loaded mDCs at a ratio of 5:1 (Fraction II Cells: Dendritic Cells (DC)) in T Cell Growth Media along with IL-21 (100 IU/mL). The co-culture cells are incubated for 3 days in 37° C., 5% CO₂ for the starvation period.

Day 6: Differentiation and Expansion of Anti-Viral Veto Cells (Tcm)

Following the 72 hour starvation phase, a sample from the supernatant of the G-Rex®100 is carefully removed without disrupting the cell layer, to determine the pH and glucose level of the culture. The pH should be at the physiologic range (pH 7.2-7.6) and the glucose at least 50 mg/dl. Fresh T cell growth medium supplemented with IL-7 (30 IU/mL), IL-15 (125 IU/mL) and IL-21 (100 IU/mL) is added to each G-Rex®100 at 50% of the culture volume. The cells are then incubated for an additional 48 hours at 37° C. with 5% CO₂. This process is repeated every 72 hours until the end of culture.

The cells are in culture for up to 12 days. Fresh media containing cytokines or only cytokines are added every 48 hours depending on the pH and glucose level. If the glucose level is between:
Glucose Level of 170 mg/dL to 130 mg/dL: only cytokines IL-7, IL-15, IL-21 are added to the culture.
Glucose Level of 129 mg/dL to 100 mg/dL: 100 ml (25% of the G-Rex®100 volume) of fresh T cell growth medium+cytokines IL-7, IL-15, IL-21 are added to the culture.
Glucose Level of 99 mg/dL to 50 mg/dL: 200 ml (50% of the G-Rex®100 volume) of fresh T cell Growth medium+cytokines IL-7, IL-15, IL-21 are added to the culture.

Of note: The maximum volume level in the G-Rex®100 is 400 mL. The volume of culture is replenished if the maximum volume in the G-Rex®100 is reached.

Day 12:

On day 12, a sterility sample is obtained for quality control release testing.

Day 13: Culture Cell Count Assessment

On Day 13 a cell count, TB viability and immunophenotyping sample is removed for TNC determination.

Day 14:

On Day 14, fresh media containing cytokines or only cytokines is added depending on the pH and glucose level as described above. Once media and/or cytokines are added the cell culture is incubated for 24 hours (+/−2 hours) at 37° C., 5% CO₂ prior to harvest.

Total Time in culture: 12 days (Day 3 to Day 15)

Day 15: Final Formulation and Infusion of Anti-Viral CD8 Central Memory Cells (Tcm)

On Day 15, the Tcm cells, inspected for visual contamination, harvested and washed with cell suspension media (Plamalyte-A/0.5% of 25% HSA). Samples are removed for non-release testing: cell count, TB viability, sterility and release-testing: endotoxin and mycoplasma prior to wash of the cells.

After the cell preparation of one embodiment is washed and resuspended, release testing samples for cell count, viability, and immunophenotyping are removed. Following the cell count and TNC determination, the cell preparation of one embodiment is resuspended in approximately 50-100 mL of cell suspension media to a Tcm cell dose according to the dose-prescribed in the clinical protocol. Samples for release testing are obtained for gram stain and sterility as non-release testing. The final Tcm cell preparation meets release criteria prior to release of the cell preparation.

The final Tcm cells are transported from the CTL to the patient floor by the CTL staff in a plastic cooler to protect the cell preparation from temperature fluctuations and physical damage. The CTL Staff deliver and issue the cell preparation to infusing personnel.

Generation of the CD34⁺ Enriched Cells

Procedure Overview

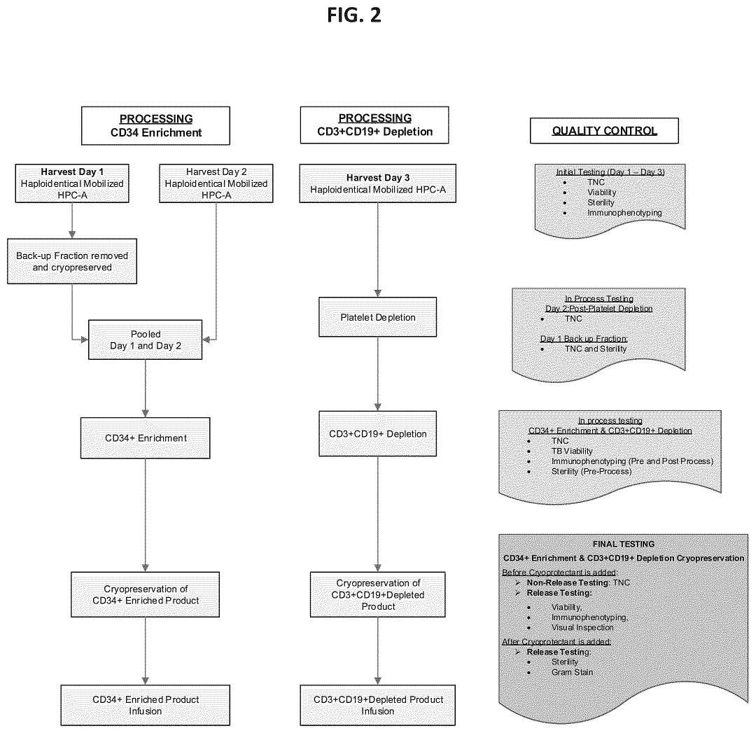

As illustrated in FIG. 2, the mobilized hematopoietic progenitor cells (also termed HPC-A) is collected in three days. A back-up fraction of unmodified PBMC containing 1×10⁶ CD34⁺ cells per kg ideal body weight is set aside and cryopreserved. The remaining first and second day's collection are pooled, CD34⁺ selected and cryopreserved. The third day's collection is depleted of CD3⁺/CD19⁺ cells and cryopreserved. The maximum T cell dose in the entire T cell depleted stem cell transplant collected from all fractions is 2×10⁵ CD3⁺ cells per kg ideal body weight.

Both procedures are performed in the CTL Core Laboratory using the Miltenyi CliniMACS® device. The CTL has well established SOPs for the use of this device and has used it in the manufacture of cell preparations for multiple clinical trials. Single HPC-A preparations may be split or pooled depending on cell number and scheduling. Cell preparations of one embodiment are cryopreserved at the completion of the respective procedure.

CD34⁺ Enrichment Process

Setup

On the first day collection of the mobilized HPC-A, the CliniMACS® PBS/EDTA Buffer bags (1000 ml) containing 0.5% Human Serum Albumin (HSA) are prepared prior to processing according to well-established laboratory SOP. The weight of the HPC-A preparation is determined. Samples from Day 1 and Day 2 of the HPC-A are removed for the following tests: cell count, viability, sterility, immunophenotyping. Each buffer and cell preparation bag is labeled with Patient's name, MDACC number, and date of preparation. A back-up fraction of 2×10⁶ CD34⁺ cells per kg ideal body weight is removed from the first day's collection before the CD34⁺ selection procedure is performed on the remaining PBPC cell preparation. The remaining cells of the first day collection are kept overnight and pooled with the second day collection for the CD34⁺ enrichment process.

CD34⁺ Isolation

The pooled HPC-A cell preparations of one embodiment are incubated with the CD34 reagent (CD34 antibody conjugated to super-paramagnetic particles). The cells are washed to remove excess reagent. Cell count and immunophenotyping samples are removed. The CD34 labeled cells are then processed on the CliniMACS® using the CliniMACS® tubing set and the CD34⁺ selection program. In this case, the magnetically labeled cells (CD34⁺) are retained by the column and CD34 negative cells are removed. The CD34⁺ cells are then released from the column and collected. Samples from each fraction are removed for cell count, viability and immunophenotyping.

The CD34⁺ enriched cell preparation may be cryopreserved for infusion (as discussed below) or may be used as fresh cells (as discussed above).

Limitations:

The maximum TNC to process with one reagent kit and tubing set is 6×10¹⁰ unless higher number is approved by the Laboratory Director or designee. The maximum load volume for the CliniMACS® instrument is 300 ml.

CD3⁺/CD19⁺ Depletion Process

Setup

CliniMACS® PBS/EDTA Buffer bags (1000 ml) containing 0.5% Human Serum Albumin (HSA) is prepared prior to processing according to well-established laboratory SOP. The weight of the HPC-A cell preparation is determined. Samples from the HPC-A are removed for the following tests: cell count, viability, sterility, and immunophenotyping. Each buffer and cell preparation bag is labeled with Patient's name, Medical Record Number (MRN), and date of preparation.

Platelet Depletion

The HPC-A cell preparation of one embodiment is platelet depleted prior to the CD3⁺/CD19⁺ depletion using the COBE 2991. Once the cells are removed from the COBE 2991, samples for cell count are removed. The cell preparation is then incubated with the CD3 reagent and CD19 reagent.

CD3⁺/CD19⁺ Depletion

The post-platelet depletion HPC-A cell preparation of one embodiment is incubated with IVIg for 10-15 minutes. After the initial incubation the CD3⁺ and CD19⁺ reagents (CD3 and CD19 antibodies conjugated to super-paramagnetic particles) are added to the cell preparation and incubated for 30 minutes on an orbital rotator. At the end of the incubation, the cell are washed using the COBE 2991 to remove excess reagent. Cell count and immunophenotyping samples are removed. The CD3⁺/CD19⁺ labeled cells are then processed on the CliniMACS® using the depletion tubing set and the depletion program. In this case, the magnetically labeled cells (CD3⁺/CD19⁺) are retained by the column and CD3 and CD19 negative cells pass through the column and are collected as a CD3⁺/CD19⁺ depleted fraction. Samples from each fraction are removed for cell count, viability and immunophenotyping.

The CD3⁺/CD19⁺ depleted cell preparation of one embodiment is cryopreserved for infusion (as discussed below) or may be used as fresh cells (as discussed above).

Limitations: The maximum TNC to process with one reagent kit and tubing set is 8×10¹⁰ unless higher number is approved by the Laboratory Director or designee.

2^{nd} CD3⁺ Depletion (not a Required Step, to be Used as Needed)

If more than 2×10⁵ CD3/kg ideal body weight are present, a second cycle of CD3 depletion may be performed on the CD3⁺/CD19⁺ depleted to further reduce the CD3 population in the cell preparation. Samples for cell counts and immunophenotyping, viability and sterility are obtained from each fraction.

Alternatively infusion of only part of the T cell depleted fraction is utilized so as to avoid infusion of more than 2×10⁵ CD3 cells/kg ideal body weight.

Final Formulation and Cryopreservation of CD34⁺ Enriched and CD3⁺/CD19⁺ Depleted Cell Preparations:

After cell selection or depletion and sampling, the cell preparations of one embodiment are concentrated and cryopreserved according to procedures validated at the CTL. A final cell count, viability, immunophenotyping, gram stain and sterility sample is obtained prior to cryopreservation of the cell preparation. Following the cell count and TNC determination, the cell preparation of one embodiment is cryopreserved in freeze media (50% Plasma-Lyte A, 7.5% DMSO, and 35% HSA 25%) according to SOP. Bags containing the CD34⁺ Enriched cells or CD3⁺/CD19⁺ Depleted cells are frozen in a controlled rate freezer and stored in a liquid nitrogen freezer in vapor phase.

Of note: The final cell preparation is the preparation before cryopreservation as the thawing of the cells occurs at bedside without further manipulation.

Cell Preparation Release and Infusion

On the day of infusion, the CD34⁺ Enriched cells and/or CD3⁺/CD19⁺ Depleted cell preparations are delivered to the patient floor by the CTL staff and issued to the infusing personnel. The final cell preparations meet lot release criteria prior to release of the cell preparations. These cells are then infused into the patient according to protocol specific dose. Of note, when cryopreserved are used, the cells are thawed on the floor and released to infusing personnel per standard operating procedure. Also, when cryopreserved are used, the final cell preparations are transported from the CTL to the floor by CTL staff in an approved styrofoam LN2 transportation container with seamless metal inserts that are be filled with approximately 2 inches of liquid nitrogen.

All cell preparation testing is protocol specific and preformed according to SOPs.

Reagents

TABLE 3
List of reagents, manufacturers, status (Licensed,
Certificate of Analysis (CoA), or Master File (MF))

| Reagent | Supplier | Regulatory Status |
|---|---|---|
| Ficoll-Paque ™ Premium | GE Helthcare | Certificate of Analysis |
| | Bio-Sciences AB | |
| Dendritic Cell Medium (DC) - CellGro | CellGenix | Certificate of Analysis |
| Click's Medium (EHAA) w/o | Irvine Scientific | Certificate of Analysis |
| Mercaptoethanol, L-Glutamine | | |
| Advanced RPMI 1640 | Gibco/Life | Certificate of Analysis |
| | Technologies | |
| Acid Citrate Dextrose - Formula A | Fenwall | Licensed Product Insert |
| (ACD-A) | | |
| Human Serum AB | Gemini Bio- | Certificate of Analysis |
| | Products | |
| Plasma-Lyte A | Baxter | Licensed Product Insert |
| Human Serum Albumin (HSA), 25% | Baxter | Licensed Product Insert |
| Dimethyl Sulfoxide (DMSO) | OriGen | Certificate of Analysis |
| | Biomedical | |
| Dulbecco's Phosphate Buffered Saline | Mediatech, | Certificate of Analysis |
| (DPBS) w/o Ca⁺² and Mg⁺² | Inc./Corning | |
| MACS GMP Peptivator AdV5 Hexon | Miltenyi Biotec | Certificate of Analysis |
| MACS GMP Peptivator HCMV pp65 | Miltenyi Biotec | Certificate of Analysis |
| MACS GMP Peptivator BKV LT | Miltenyi Biotec | Certificate of Analysis |
| MACS GMP Peptivator EBV Select | Miltenyi Biotec | Certificate of Analysis |
| CliniMACS ® CD4 Reagent System | Miltenyi Biotec | Cross Reference Letter |
| | | Certificate of Analysis |
| CliniMACS ® CD56 Reagent System | Miltenyi Biotec | Cross Reference Letter - |
| | | Certificate of Analysis |
| CliniMACS ® CD45RA Reagent System | Miltenyi Biotec | Cross Reference Letter - |
| | | Certificate of Analysis |
| CliniMACS ® CD14 Reagent System | Miltenyi Biotec | Cross Reference Letter - |
| | | Certificate of Analysis |
| CliniMACS ® CD34 Reagent System | Miltenyi Biotec | Cross Reference Letter - |
| | | Certificate of Analysis |
| CliniMACS ® CD3 Reagent System | Miltenyi Biotec | Cross Reference Letter - |
| | | Certificate of Analysis |
| CliniMACS ® CD19 Reagent System | Miltenyi Biotec | Cross Reference Letter - |
| | | Certificate of Analysis |
| CliniMACS ® PBS/EDTA Buffer | Miltenyi Biotec | Cross Reference Letter - |
| | | Certificate of Analysis |
| LPS VacciGrade | InvivoGen | Certificate of Analysis |
| Interferon-Gamma (IFN-γ) | CellGenix | Certificate of Analysis |
| Interleukine -4 (IL-4) | CellGenix | Certificate of Analysis |
| Interleukine -7 (IL-7) | CellGenix | Certificate of Analysis |
| Interleukine -15 (IL-15) | CellGenix | Certificate of Analysis |
| Interleukine -21 (IL-21) | CellGenix | Certificate of Analysis |
| GM-CSF | CellGenix | Certificate of Analysis |
| Gammagard | Baxter | Licensed Product Insert |
| XS Columns | Miltenyi Biotec | Certificate of Analysis |
| CliniMACS ® Depletion Tubing Set | Miltenyi Biotec | Certificate of Analysis |

Reagents are protocol specific and stored according to manufacturer's recommendations. All reagents are qualified prior to the initiation of cell preparation manufacturing. The inventory control of reagents includes documentation of receipt, reagent documentation review, tracking by the inventory manager; and review and release by QA as appropriate. Documentation for all FDA approved reagents is available through the MDACC Pharmacy. Certificates of Analysis and Cross-reference Letters for unapproved reagents must be obtained from the supplier. MSDS are managed as part of the Laboratory Safety Policy.

Qualification Program

Reagents must be qualified prior to the initiation of production of clinical preparations. This is typically accomplished during the validation of the procedures involved in manufacture. Reagents and vendors may have previously been qualified as part of a previous clinical protocol.

Removal of Reagent from the Final Cell Preparation

The reagents are removed from the final cell preparation. This is accomplished using various cell washing, concentrating, and re-suspension procedures.

Testing of Cell Preparations

Microbiological Testing

Sterility Testing:

BD BACTEC: The MDACC Department of Laboratory Medicine tests In-Process and final cell preparation samples using this automated microbial detection system. All test are held for 14 days or until the cell preparation becomes positive. Test results are obtained before release of the cell preparation.

Samples to be Tested:

The number and timing of testing is a factor of the amount of manipulations performed on the cells, culture time, and test requirements. The samples to be tested must be optimal for the type of test. Sample conditions are validated prior to the initiation of manufacturing to insure that no part of the culture media or other buffers interfere with the sterility assay. A minimum sample volume of 0.5 ml/test is required.

Sterility Sampling Points:

Anti-viral Central Memory CD8⁺ Veto T Cells
1. Day 1: HPC-A—Pre-Ficoll
2. Day 1: HPC-A—Post Ficoll-Fraction I
3. Day 1: CD14⁺ Isolation—Negative Fraction (overnight)
4. Day 1: CD14⁺ Isolation—Positive Fraction with cytokines (overnight)
5. Day 2: CD4⁺CD56⁺CD45RA⁺ Depletion—Negative fraction (Fraction II) (overnight)
6. Day 3: Viral Loaded DCs—Post irradiation
7. Day 3: Fraction II—Overnight Harvest
8. Day 3: Co-Culture—Fraction II and Viral Loaded DCs
9. Day 12: Co-Culture—Day 12
10. Day 13: Co-Culture—Day 13
11. Day 15: Co-Culture—Post Harvest
12. Day 15: Final Tcm Cells

CD34⁺ Megadose
1. HPC-A—Harvest
2. CD34⁺ Enrichment Cells—Final cell preparation (Pre-cryo) or used fresh
3. CD3⁺CD19⁺ Depleted Cells—Final cell preparation (Pre-cryo) or used fresh

Mycoplasma Testing

The cryopreserved Tcm cell preparation of one embodiment is tested for the presence of Mycoplasma. The Tcm cell preparation (cryopreserved) is tested by a Gel Endpoint Polymerase (PCR) based assay. The PCR assay is performed by an approved licensed reference laboratory. In addition to the PCR based assay, the final Tcm cells are also tested using an approved biochemical mycoplasma rapid detection test (MycoAlert, Lonza Walkersville, Inc.). The sample is taken at time of harvest before cell washing and contains both cell and media. The sample volume is a minimum of 0.5 ml. The MycoAlert result is used as an interim test release criteria while the mycoplasma PCR based assay is performed. The mycoplasma PCR result is not used as a release criteria but is analyzed along with clinical responses to help identify criteria associated with positive clinical outcomes. Mycoplasma result must be Negative.

Positive Sterility Results Received Post Preparation Infusion

In the event the 14 day sterility or mycoplasma results on the cultured cell preparation return positive after the cell preparation has been infused, the Laboratory Director, Technical Director, Medical Director, Laboratory Quality Assurance, Principle Investigator, Attending Physician and the Investigational New Drug (IND) Office—or the Institutional Sponsor/VP of Clinical Research is notified. Quality Assurance is conducted an investigation to identify any corrective and/or preventative actions. The investigation and proposed corrections for a positive sterility testing includes: identification of the microorganism(s) and anti-microbial agent sensitivity testing; evaluation of the current procedure for collecting and processing the cell preparation to determine the step at which contamination could be introduced; development of changes in procedures that assist in preventing sterility lapses in the future; and implementation of appropriate testing to ensure that such changes to procedures produce a sterile cell preparation. The Attending Physician is responsible for notification, monitoring and treatment of the patient. The Principal Investigator is responsible for notifying the appropriate regulatory agencies (FDA, IRB) of deviations and adverse events. Additionally, the sterility failure and result of investigation of the cause and any corrective actions is reported (in an information amendment submitted to the IND in a timely manner, e.g. within 30 calendar days after initial receipt of the positive culture test result).

Gram Stain

In situations where a rapid microbial test is required, a Gram Stain is performed by the MDACC Laboratory Medicine Department. A minimum of 0.5 ml of sample is required. The Tcm cells, CD34⁺ enriched cell preparation and CD3⁺CD19⁺ Depleted cell preparation are tested and results are part of the release criteria for fresh infusion. Cell preparations must have no organisms seen.

Identity

All CTL cell preparations are labeled and stored as to ensure accurate identification and prevent mix-ups. All cell preparations are assigned a unique identifier number. Critical steps during cell preparation labeling and retrieval require verification by supervising personnel.

CD34⁺ Enriched and CD3⁺/CD19⁺ Depleted Cell Preparations: Flow cytometry is also used to identify the different cell populations that are infused back into the patient. In addition to CD34⁺ and CD3⁺/CD19⁺ determination, the following subset markers are evaluated: CD3, CD4, CD8, CD16/CD56, CD19 CD14, CD45RO and CD37.

Tem Cell Preparations: Flow cytometry is also used to identify the different cell populations that are infused back into the patient. In addition to CD45, CD3, CD8, CD62L, and CD45RO determination the following subset markers are evaluated: CD4, CD56, CD16, CD95 and CD45RA.

Purity

Flow cytometry is used at multiple points to determine the cellular phenotype. The Tcm Cell Preparation Flow cytometry marker for CD3⁺CD8⁺CD62L⁺CD45RO⁺7AAD⁻ of CD45⁺ cells must be ≥30%.

The total of both CD34⁺ Enriched Preparation and CD3⁺CD19⁺ Depleted preparation must have a minimal acceptable cell dose of greater than 6×10⁶ CD34⁺ cells per kg ideal body weight.

All other immunophenotyping data are not used as release criteria but are analyzed along with clinical responses to help identify appropriate potency assay that may be associated with positive clinical outcomes.

Potency

Immunophenotyping by flow cytometry is used to measure several potential potency markers including the markers mentioned in the Purity section above. None of the other immunophenotyping data is used as release criteria but is analyzed along with clinical responses to help identify criteria associated with positive clinical outcomes.

A potency assay is developed throughout this Phase I and be established prior to the initiation of the Phase III trial.

Endotoxin

Endotoxin levels are determined in the CTL Quality Control laboratory using the Limulus Amebocyte Lysate (LAL) assay. This method is validated for all cell/media combinations to be tested prior to clinical use. The final Tcm cell preparations are tested and results are part of the release criteria. A minimum of 0.5 mL of sample is required. Endotoxin level must be less than 5 EU/Kg ideal body weight of the recipient.

Visual Inspection

All cell preparations are visually inspected prior to infusion. Cell preparations must have no evidence of contamination.

Viability

Viability can be determined by the CTL by using either the Trypan Blue (TB) assay or by using flow cytometry (7AAD). The final Tcm Cells, CD34⁺ Enriched Cell Preparations and CD3⁺CD19⁺ Depleted Preparations must have a 7AAD viability of ≥70%. Trypan blue viability may be used as in-process testing during the process.

Cell Dose

The CTL cell dose is determined using cell counting and immunophenotyping by flow cytometry.

The final Tcm cell preparation dose is determined by the CD8⁺ dose level on which the subject is enrolled for the clinical trial. Excess cells may be used for correlative studies for cell function, phenotype studies and cryopreservation. Data is not used as release criteria, but is analyzed along with clinical responses to help identify criteria associated with positive clinical outcomes. If the Tcm cell preparation CD8⁺ dose is less than the planned dose level one (1) of 2.5×10⁶ CD8⁺ cells per kg ideal body weight (dose level 1) or if the CD8⁺ cells do not meet release criteria, these cells are not infused. Instead, the backup PBPC plus the T cell depleted PBPC is infused to reconstitute a “standard PBPC transplant” and the patient receives a standard of care preparative regimen per protocol. If the Tcm cells meet release criteria, but the CD8⁺ cell dose is less than the target cell dose level of dose level 2 of 5×10⁶ CD8⁺ cells per kg ideal body weight then 2.6 to 5×10⁶ CD8⁺ cells per kg ideal body weight is considered adequate and considered dose level 2. For dose level 3 with a target dose of 10×10⁶ CD8⁺ cells per kg ideal body weight then 5.1 to 10×10⁶ CD8⁺ cells per kg ideal body weight is considered adequate, and considered dose level 3.

The total of both the CD34⁺ Enriched Preparations and CD3⁺CD19⁺ Depleted preparations must have a minimal acceptable cell dose of 6×10⁶ CD34⁺ cells per kg ideal body weight and not to exceed the limits of T cell dose defined by the release criteria as 2×10⁵ CD3⁺ cells per kg ideal body weight. The final CD34⁺ Enriched cell preparation dose is monitored and analyzed along with clinical responses to help identify criteria associated with positive clinical outcomes.

Retain Aliquot

All QC tubes are retained for one week; however the usefulness of these samples decreases rapidly after 48 hours for most samples. All frozen cell preparations also have reference vials simultaneously stored at the time of cryopreservation.

Cell Preparation Release Criteria Testing and Additional Testing

TABLE 4
Tcm Preparation Lot Release testing and Specifications

| Test | Specifications |
|---|---|
| Viability (7AAD) | ≥70% |
| Visual Inspection | No evidence of contamination |
| Gram Stain | No organism seen |
| Sterility (Day 12) | Negative-to-Date |
| Mycoplasma | Negative |
| (MycoAlert) | |
| Endotoxin (LAL) | <5 EU/Kg ideal body weight |
| Immunophenotyping | ≥30% CD3⁺CD8⁺CD62L⁺CD45RO⁺7AAD⁻ |
| | of CD45⁺ cells |
| CD8⁺ Cell Dose | Dose 1: 2.5 × 10⁶ CD8⁺/kg ideal body weight |
| | Dose 2: ≤5 × 10⁶ CD8⁺/kg ideal body weight |
| | but ≥2.6 × 10⁶ CD8⁺/kg ideal body weight |
| | Dose 3: ≤10 × 10⁶ CD8⁺/kg ideal body weight |
| | but ≥5.1 × 10⁶ CD8⁺/kg ideal body weitght |

TABLE 5
CD34⁺ Enriched Preparation Lot Release testing and Specifications

| Test: | Specifications: |
|---|---|
| Sterility | No Growth at 14 Days |
| Gram Stain | No Organisms Seen |
| Viability (Final Preparation) | ≥70% |
| Total CD3⁺ Cell Dose | <2 × 10⁵ CD3⁺ cells per kg ideal |
| | body weight |
| Total CD34⁺ Cell Dose | ≥5 × 10⁶ CD34⁺ cells per kg ideal |
| | body weight |

TABLE 6
Additional In-Process Test (non-release)

| Test: | Specification |
|---|---|
| Sterility | No Growth at 14 Days |
| Immunophenotype | For Information Only |
| TNC | For Information Only |
| Mycoplasma (PCR) | Negative |

### Example 3

Treatment Regimen in the Absence of ATG

Materials and Experimental Conditions

Preparation of Host Non-Reactive Donor Anti 3^{rd}-Party Cells

Anti-3^{rd}-party Tem cells were prepared as previously described [Ophir E. et al., Blood. 2013, 121:1220]. Briefly, splenocytes of the donor mice were cultured against irradiated 3rd-party splenocytes for 60 hours under cytokine deprivation. Subsequently, CD8 cells were positively selected using Magnetic Particles (BD Pharmingen) and cultured in an Ag-free environment. rhIL-15 (20 ng/mL; R&D Systems) was added every second day. To attain a purified population at the end of the culture (day 16), the Tcms were positively selected for CD62L expression (magnetic-activated cell sorting [MACS® Cell Separation, Miltenyi Biotec, Bergisch Gladbach, Germany]), and cells were retrieved for FACS analysis.

Preparation of Hematopoietic Stem Cells

Long bones were harvested from Balb/c-Nude mice (10-12 weeks of age). Bone marrow was extracted by grinding the bones to reach a single cell suspension. Bone marrow was counted and brought to the correct concentration and was then injected to mice IV to the tail vein.

Chimerism Analysis

Chimerism was determined by flow cytometry. Peripheral blood was collected by retro-orbital bleeding, cells were fractionated on Ficoll-Paque Plus (Amersham Pharmacia Biotech, AB), and the isolated mononuclear cells of each mouse were stained by direct immuno-fluorescence against donor and host surface markers.

Anti-Viral Activity of Tcm Cells as Determined by Intracellular Staining

Veto-Tcm cells were co-cultured with the respective viral peptide mix (EBV, CMV, Adeno, BKV) in the presence of Brefeldin A (eBioscience) at 37° C., 5% CO₂ for 6 hrs. Cells were fixed, permeabilized (Invitrogen Fix & Perm set), and immunostained for CD45, CD3, CD8, IFN-γ, and TNF-α (BD). Positive control included TCR-independent stimulation with PMA/Ionomycin. Cells were gated on a CD45⁺/FSC lymphocyte gate and CD3⁺CD8⁺.

Results

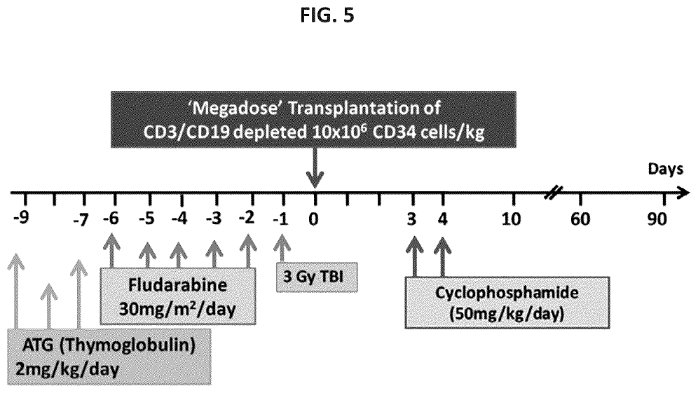

The present inventors have previously demonstrated that combing T cell depleted megadose haplo-HCT with post-transplant CY following a non-myeloablative conditioning by T cell debulking with ATG, 3 GY TBI and Fludarabine (FIG. 5) can enable durable engraftment in the absence of GVHD (data not shown).

Considering that T cell debulking with anti-T cell antibodies such as ATG is associated with significant toxicities including enhanced risk for viral infections, the possibility of replacing T cell debulking by the use of central memory veto cells, infused on day 7 was examined.

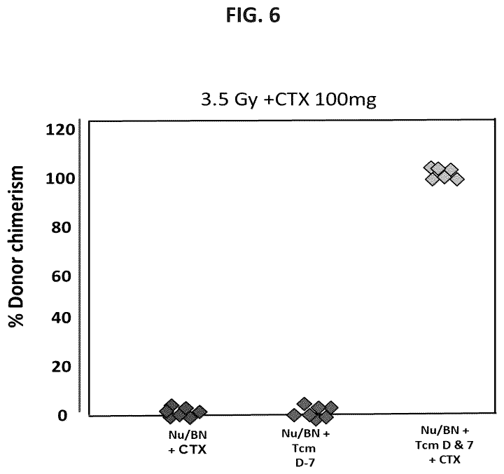

As shown in FIG. 6, combining T cell depleted megadose haplo-HCT with CY post-transplant following sub-lethal TBI (3.5 GY) in the absence of T cell debulking, resulted in transplant rejection and no evidence of chimerism. In contrast, infusion of central memory CD8⁺ veto cells on day 7 enabled marked donor type chimerism.

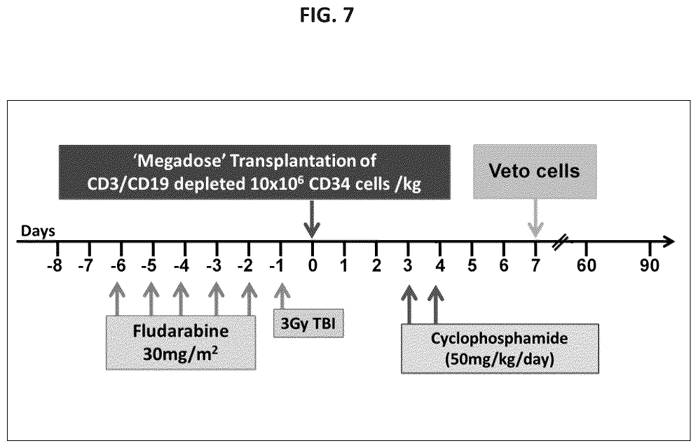

These results provide a proof of concept for using veto cell administration on day 7 as a replacement for T cell debulking with agents such as ATG. Such a protocol includes sub-lethal TBI (e.g. 3 GY), Fludarabine, megadose T cell depleted hematopoietic stem cells and CY post-transplant as illustrated in FIG. 7.

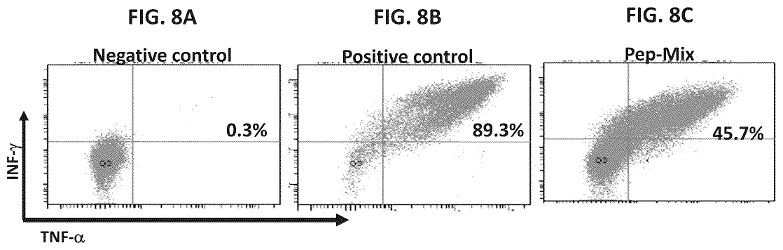

Furthermore, the veto cell product uniquely exhibited anti-viral activity directed against a mix of viral peptides, i.e. EBV, CMV, Adeno, BKV, as illustrated by INF-γ and TNF-α staining (FIGS. 8A-C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of generating an isolated population of non-graft versus host disease (GVHD) inducing cells comprising a central memory T-lymphocyte (Tcm) phenotype, said cells being tolerance inducing cells and/or endowed with anti-disease activity, and capable of homing to the lymph nodes following transplantation, the method comprising:
(a) contacting a first population of peripheral blood mononuclear cells (PBMCs) from a donor subject with an antibody capable of binding CD14⁺ expressing cells and selecting CD14⁺ expressing cells capable of maturing into antigen presenting cells;
(b) loading said antigen presenting cells with viral peptides;
(c) treating a second population of PBMCs of the same donor subject as said first population of PBMCs with one or more agents capable of depleting CD4⁺, CD56⁺ and CD45RA⁺ expressing cells so as to obtain a population of cells comprising memory T cells expressing a CD45RA⁻CD8⁺ phenotype;
(d) contacting said population of cells comprising said memory T cells with said antigen presenting cells loaded with said viral peptides of step (b) in the presence of IL-21 so as to allow enrichment of viral reactive memory T cells; and
(e) culturing said cells resulting from step (d) in the presence of IL-21, IL-15 and/or IL-7 so as to allow proliferation of cells comprising said Tcm phenotype, thereby generating the isolated population of non-GVHD inducing cells comprising said Tcm phenotype.

2. A method of generating an isolated population of non graft versus host disease (GVHD) inducing cells comprising a central memory T-lymphocyte (Tcm) phenotype, said cells being tolerance inducing cells and/or endowed with anti-disease activity, and capable of homing to the lymph nodes following transplantation, the method comprising:
(a) providing a population of cells comprising T cells, wherein said T cells in said population of cells comprise at least 40% memory T cells expressing CD45RA⁻CD8⁺ phenotype and depleted of CD4⁺, CD56⁺ and CD45RA⁺ expressing cells; and
(b) contacting said population of cells comprising said memory T cells with viral peptides derived from 4-10 types of viruses, wherein at least one of said viruses comprises a BK virus, in the presence of IL-21 so as to allow enrichment of viral reactive memory T cells; and
(c) culturing said cells resulting from step (b) in the presence of IL-21, IL-15 and/or IL-7 so as to allow proliferation of cells comprising said Tcm phenotype, thereby generating the isolated population of non-GVHD inducing cells comprising said Tcm phenotype.

3. A method of generating an isolated population of non-graft versus host disease (GVHD) inducing cells comprising a central memory T-lymphocyte (Tcm) phenotype, said cells being tolerance inducing cells and/or endowed with anti-disease activity, and capable of homing to the lymph nodes following transplantation, the method comprising:
(a) contacting a first population of peripheral blood mononuclear cells (PBMCs) from a donor subject with an antibody capable of selecting CD14⁺ expressing cells and selecting CD14⁺ expressing cells capable of maturing into dendritic cells;
(b) loading said dendritic cells with viral peptides derived from 4-10 types of viruses, wherein at least one of said viruses comprises a BK virus;
(c) treating a second population of PBMCs of the same donor subject as said first population of PBMCs with one or more agents capable of depleting CD4⁺, CD56⁺ and CD45RA⁺ expressing cells so as to obtain a population of cells comprising memory T cells expressing a CD45RA⁻CD8⁺ phenotype;
(d) contacting said population of cells comprising said memory T cells with said dendritic cells loaded with said viral peptides of step (b) in the presence of IL-21 so as to allow enrichment of viral reactive memory T cells; and
(e) culturing said cells resulting from step (d) in the presence of IL-21, IL-15 and/or IL-7 so as to allow proliferation of cells comprising said Tcm phenotype, thereby generating the isolated population of non-GVHD inducing cells comprising said Tcm phenotype.

4. The method of claim 1, wherein said first population of said PBMCs and said second population of said PBMCs are from the same batch.

5. The method of claim 1, wherein said memory T cells expressing said CD45RA⁻CD8⁺ phenotype constitute at least 40% of T cells in said population of cells.

6-9. (canceled)

10. The method of claim 1, wherein said culturing further comprises adding glucose to a concentration of at least 50 mg/dl.

11. The method of claim 2, wherein said viral peptides are presented on antigen presenting cells.

12. The method of claim 1, wherein said antigen presenting cells are of the same donor subject as said population of cells comprising said memory T cells.

13-14. (canceled)

15. The method of claim 1, wherein said viral peptides are derived from 4-10 types of viruses.

16. The method of claim 1, herein said viral peptides comprise at least one peptide from a BK virus.

17. The method of claim 2, wherein said viral peptides comprise an Epstein-Barr virus (EBV) peptide, a cytomegalovirus (CMV) peptide, a BK Virus peptide and Adenovirus (Adv) peptide.

18. The method of claim 2, wherein said viral peptides comprise at least one of EBV-LMP2, EBV-BZLF1, EBV-EBNA1, EBV select, CMV-pp65, CMV-IE-1, Adv-penton, Adv-hexon, BKV LT, BKV (capsid VP1), BKV (capsid protein VP2), BKV (capsid protein VP2, isoporm VP3) and BKV (small T antigen).

19. The method of claim 2, wherein said viral peptides comprise at least one of AdV5 Hexon, hCMV pp65, EBV select and BKV LT.

20-23. (canceled)

24. An isolated population of non-GVHD inducing cells comprising cells having a central memory T-lymphocyte (Tcm) phenotype, said cells being tolerance inducing cells and/or endowed with anti-disease activity, and capable of homing to the lymph nodes following transplantation, generated according to the method of claim 1.

25. A pharmaceutical composition comprising as an active ingredient the isolated population of non-GVHD inducing cells of claim 24 and a pharmaceutical acceptable carrier.

26. A method of treating a disease in a subject in need thereof, the method comprising administering to the subject a therapeutically effective amount of the isolated population of non-GVHD inducing cells of claim 24, thereby treating the disease in the subject.

27. (canceled)

28. The method of claim 26, further comprising transplanting a cell or tissue transplant into the subject.

29. (canceled)

30. The method of claim 26, further comprising administering to the subject genetically modified T cells or immature hematopoietic cells.

31-35. (canceled)

36. A method of reducing graft rejection and/or graft versus host disease (GVHD) and/or inducing donor specific tolerance in a subject in need thereof, wherein said subject is in need of a non-syngeneic cell or tissue transplant, the method comprising administering to the subject a therapeutically effective amount of the isolated population of non-GVHD inducing cells of claim 24, thereby reducing graft rejection and/or GVHD in the subject.

37. (canceled)

38. A method of treating a subject in need of a non-syngeneic cell or tissue transplant, the method comprising:
(a) transplanting a cell or tissue transplant into the subject; and
(b) administering to the subject a therapeutically effective amount of the isolated population of non-GVHD inducing cells of claim 24, thereby treating the subject in need of the cell or tissue transplant.

39. (canceled)

40. The method of claim 28, wherein said isolated population of non-GVHD inducing cells is for administration following said cell or tissue transplant and/or is for administration at a dose of at least 2.5×10⁶ CD8⁺ cells per kg ideal body weight.

41-44. (canceled)

45. The method of claim 28, further comprising:
conditioning the subject under non-myeloablative conditioning protocol prior to said transplanting; and/or
administering to the subject a therapeutically effective amount of cyclophosphamide subsequent to said transplanting.

46-49. (canceled)

50. The method of claim 45, wherein said therapeutically effective amount of said cyclophosphamide:
comprises 25-200 mg cyclophosphamide per kilogram ideal body weight of the subject; and/or
is to be administered to the subject in two doses 3 and 4 days post-transplant.

51. A method of treating a subject in need of an immature hematopoietic cell transplantation, the method comprising:
(a) conditioning the subject under non-myeloablative pre-transplant conditioning protocol, wherein said non-myeloablative conditioning comprises a total body irradiation (TBI) and a chemotherapeutic agent, wherein said TBI and said chemotherapeutic agent are administered on days −7 to −1 prior to transplantation;
(b) transplanting into the subject a dose of T cell depleted immature hematopoietic cells, wherein said T cell depleted immature hematopoietic cells comprises less than 5×10⁵ CD3⁺ T cells per kilogram ideal body weight of the subject, and wherein said T cell depleted immature hematopoietic cells comprise at least 5×10⁶ CD34⁺ cells per kilogram ideal body weight of the subject;
(c) administering to the subject a therapeutically effective amount of cyclophosphamide, wherein said therapeutically effective amount of said cyclophosphamide comprises 25-200 mg cyclophosphamide per kilogram ideal body weight of the subject, and wherein said therapeutically effective amount of said cyclophosphamide is to be administered to the subject in two doses 3 and 4 days following said transplantation of said T cell depleted immature hematopoietic cells; and
(d) administering to the subject a therapeutically effective amount of the isolated population of non-GVHD inducing cells of claim 24, wherein said isolated population of non-GVHD inducing cells are administered on day 6-9 following said transplantation of said T cell depleted immature hematopoietic cells, thereby treating the subject in need of the immature hematopoietic cell transplantation.

52. (canceled)

53. The method of claim 45, wherein the subject is not treated chronically with GVHD prophylaxis following transplantation and/or wherein corticosteroids are not administered.
